SEQUENCE LISTING (36 sequences)

SEQ ID NO: 1 - protein, 5 aa, Artificial sequence
  HCDR1
RYWMS

SEQ ID NO: 2 - protein, 15 aa, Artificial Sequence
  HCDR2
EINPDSSTINYTSSL

SEQ ID NO: 3 - protein, 7 aa, Artificial Sequence
  HCDR3
LITTEDY

SEQ ID NO: 4 - protein, 11 aa, Artificial Sequence
  LCDR1
KASQDINSYLS

SEQ ID NO: 5 - protein, 7 aa, Artificial Sequence
  LCDR2
YANRLVD

SEQ ID NO: 6 - protein, 9 aa, Artificial Sequence
  LCDR3
LQYDEFPYT

SEQ ID NO: 7 - protein, 116 aa, Artificial Sequence
  VH region
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCAILITTEDYWGQGTTVTVSS

SEQ ID NO: 8 - protein, 107 aa, Artificial Sequence
  VL region
DIQLTQSPSSLSASVGDRVTITCKASQDINSYLSWFQQKPGKAPKSLIYYANRLVDGVPSRFSGSGSGTDFTLTISSLQPEDFATYYCLQYDEFPYTFGGGTKVEIK

SEQ ID NO: 9 - protein, 5 aa, Artificial Sequence
  HCDR1
RSWIS

SEQ ID NO: 10 - protein, 11 aa, Artificial Sequence
  LCDR1
KASQDINKYLS

SEQ ID NO: 11 - protein, 9 aa, Artificial Sequence
  LCDR3
LQYDVFPYT

SEQ ID NO: 12 - protein, 116 aa, Artificial Sequence
  VH region
EVQLVESGGGLVQPGGSLRLSCAVSGFVFSRSWISWVRQAPGKGLEWIGEINPDSSTINYTSSLKDRFTISRDNAKNSLYLQMNSLRAEDTAVYYCAILITTEDYWGQGTTVTVSS

SEQ ID NO: 13 - protein, 107 aa, Artificial Sequence
  VL region
DIQLTQSPSSLSASVGDRVTITCKASQDINKYLSWFQQKPGKAPKSLIYYANRLVDGVPSRFSGSGSGTDFTLTISSLQPEDFATYYCLQYDVFPYTFGGGTKVEIK

SEQ ID NO: 14 - DNA, 348 bp, Artificial Sequence
  VH region
gaggtgcagctggtggaaagcggcggaggactggtgcagcctggcggcagcctgagactgagctgcgccgtgtccggcttcgtgttcagccggagctggatcagctgggtccgccaggccccagggaagggcctggaatggatcggcgagatcaaccccgacagcagcaccatcaactacaccagcagcctgaaggaccggttcaccatcagccgggacaacgccaagaacagcctgtacctgcagatgaacagcctgcgggccgaggacaccgccgtgtactactgcgccatcctcatcaccaccgaggactactggggccagggcaccaccgtgaccgtgtcctct

SEQ ID NO: 15 - DNA, 321 bp, Artificial Sequence
  VL region
gacatccagctgacccagagccccagcagcctgagcgccagcgtgggcgacagagtgaccatcacatgcaaggccagccaggacatcaacaagtacctgagctggttccagcagaagcccggcaaggcccccaagagcctgatctactacgccaaccggctggtggacggcgtgcccagcagattttctggcagcggcagcggcaccgacttcaccctgaccatcagcagcctgcagcccgaggacttcgccacctactactgcctgcagtacgacgtgttcccctacaccttcggcggaggcaccaaggtggaaatcaag

SEQ ID NO: 16 - protein, 769 aa, Homo sapiens
MCGSALAFFTAAFVCLQNDRRGPASFLWAAWVFSLVLGLGQGEDNRCASSNAASCARCLALGPECGWCVQEDFISGGSRSERCDIVSNLISKGCSVDSIEYPSVHVIIPTENEINTQVTPGEVSIQLRPGAEANFMLKVHPLKKYPVDLYYLVDVSASMHNNIEKLNSVGNDLSRKMAFFSRDFRLGFGSYVDKTVSPYISIHPERIHNQCSDYNLDCMPPHGYIHVLSLTENITEFEKAVHRQKISGNIDTPEGGFDAMLQAAVCESHIGWRKEAKRLLLVMTDQTSHLALDSKLAGIVVPNDGNCHLKNNVYVKSTTMEHPSLGQLSEKLIDNNINVIFAVQGKQFHWYKDLLPLLPGTIAGEIESKAANLNNLVVEAYQKLISEVKVQVENQVQGIYFNITAICPDGSRKPGMEGCRNVTSNDEVLFNVTVTMKKCDVTGGKNYAIIKPIGFNETAKIHIHRNCSCQCEDNRGPKGKCVDETFLDSKCFQCDENKCHFDEDQFSSESCKSHKDQPVCSGRGVCVCGKCSCHKIKLGKVYGKYCEKDDFSCPYHHGNLCAGHGECEAGRCQCFSGWEGDRCQCPSAAAQHCVNSKGQVCSGRGTCVCGRCECTDPRSIGRFCEHCPTCYTACKENWNCMQCLHPHNLSQAILDQCKTSCALMEQQHYVDQTSECFSSPSYLRIFFIIFIVTFLIGLLKVLIIRQVILQWNSNKIKSSSDYRVSASKKDKLILQSVCTRAVTYRREKPEEIKMDISKLNAHETFRCNF

SEQ ID NO: 17 - DNA, 8787 bp, Homo sapiens
ggcgggtgcttctagggcgctcccagagccgcctccccctgttgctggcatcccgagcttcctcccttgccagccaggacgctgccgacttgtctttgcccgctgctccgcagacggggctgcaaagctgcaactaatggtgttggcctccctgcccacctgtggaagcaactgcgctgattgatgcgccacagacttttttcccctcgacctcgccggcgtcccctcccacagatccagcatcacccagtgaatgtacattagggtggtttcccccccagcttcgggctttgtttgggtttgattgtgtttggctcttcgctaagctgatttatgcagcagaagccccaccggctggagagaaacaaaagctcttttctttgtcccggagcaggctgcggagcccttgcagagccctctctccagtcgccgccggggcccttggccgtcgaaggaggtgcttctcgcggagaccgcgggacccgccgtgccgagccgggagggccgcaggggccctgagatgccgagcggtgcccgggcccgcttacctgcaccgcttgctccgagccgcggggtccgcctgctaggcctgcggaaaacgtcctagcgacactcggcccgcgggccccgaggtgcgcccgggaggcgcgagcccgcgtccggaaggcagtcaggcggcgggcgcggggcgggctgttttgcattatgtgcggctcggccctggctttttttaccgctgcatttgtctgcctgcaaaacgaccggcgaggtcccgcctcgttcctctgggcagcctgggtgttttcacttgttcttggactgggccaaggtgaagacaatagatgtgcatcttcaaatgcagcatcctgtgccaggtgccttgcgctgggtccagaatgtggatggtgtgttcaagaggatttcatttcaggtggatcaagaagtgaacgttgtgatattgtttccaatttaataagcaaaggctgctcagttgattcaatagaatacccatctgtgcatgttataatacccactgaaaatgaaattaatacccaggtgacaccaggagaagtgtctatccagctgcgtccaggagccgaagctaattttatgctgaaagttcatcctctgaagaaatatcctgtggatctttattatcttgttgatgtctcagcatcaatgcacaataatatagaaaaattaaattccgttggaaacgatttatctagaaaaatggcatttttctcccgtgactttcgtcttggatttggctcatacgttgataaaacagtttcaccatacattagcatccaccccgaaaggattcataatcaatgcagtgactacaatttagactgcatgcctccccatggatacatccatgtgctgtctttgacagagaacatcactgagtttgagaaagcagttcatagacagaagatctctggaaacatagatacaccagaaggaggttttgacgccatgcttcaggcagctgtctgtgaaagtcatatcggatggcgaaaagaggctaaaagattgctgctggtgatgacagatcagacgtctcatctcgctcttgatagcaaattggcaggcatagtggtgcccaatgacggaaactgtcatctgaaaaacaacgtctatgtcaaatcgacaaccatggaacacccctcactaggccaactttcagagaaattaatagacaacaacattaatgtcatctttgcagttcaaggaaaacaatttcattggtataaggatcttctacccctcttgccaggcaccattgctggtgaaatagaatcaaaggctgcaaacctcaataatttggtagtggaagcctatcagaagctcatttcagaagtgaaagttcaggtggaaaaccaggtacaaggcatctattttaacattaccgccatctgtccagatgggtccagaaagccaggcatggaaggatgcagaaacgtgacgagcaatgatgaagttcttttcaatgtaacagttacaatgaaaaaatgtgatgtcacaggaggaaaaaactatgcaataatcaaacctattggttttaatgaaaccgctaaaattcatatacacagaaactgcagctgtcagtgtgaggacaacagaggacctaaaggaaagtgtgtagatgaaacttttctagattccaagtgtttccagtgtgatgagaataaatgtcattttgatgaagatcagttttcttctgagagttgcaagtcacacaaggatcagcctgtttgcagtggtcgaggagtttgtgtttgtgggaaatgttcatgtcacaaaattaagcttggaaaagtgtatggaaaatactgtgaaaaggatgacttttcttgtccatatcaccatggaaatctgtgtgctgggcatggagagtgtgaagcaggcagatgccaatgcttcagtggctgggaaggtgatcgatgccagtgcccttcagcagcagcccagcactgtgtcaattcaaagggccaagtgtgcagtggaagaggcacgtgtgtgtgtggaaggtgtgagtgcaccgatcccaggagcatcggccgcttctgtgaacactgccccacctgttatacagcctgcaaggaaaactggaattgtatgcaatgccttcaccctcacaatttgtctcaggctatacttgatcagtgcaaaacctcatgtgctctcatggaacaacagcattatgtcgaccaaacttcagaatgtttctccagcccaagctacttgagaatatttttcatcattttcatagttacattcttgattgggttgcttaaagtcctgatcattagacaggtgatactacaatggaatagtaataaaattaagtcctcatcagattacagagtgtcagcctcaaaaaaggataagttgattctgcaaagtgtttgcacaagagcagtcacctaccgacgtgagaagcctgaagaaataaaaatggatatcagcaaattaaatgctcatgaaactttcaggtgcaacttctaaaaaaagatttttaaacacttaatgggaaactggaattgttaataattgctcctaaagattataattttaaaagtcacaggaggagacaaattgctcacggtcatgccagttgctggttgtacactcgaacgaagactgacaagtatcctcatcatgatgtgactcacatagctgctgactttttcagagaaaaatgtgtcttactactgtttgagactagtgtcgttgtagcactttactgtaatatataacttatttagatcagcatagaatgtagatcctctgaagagcactgattacactttacaggtacctgttatccctacgcttcccagagagaacaatgctgtgagagagtttagcattgtgtcactacaagggtacagtaatccctgcactggacatgtgaggaaaaaaataatctggcaagtatattctaaggttgccaaacacttcaacagttggtggttgaatagacaagaacagctagatgaataaatgattcgtgtttcactctttcaagaggtgaacagatacaaccttaatcttaaaagattattgctttttaaagtgtgtagttttatgcatgtgtgtttatggtttgcttatttttgcaagatggatactaattccagcattctctcctctttgcctttatgttttgttttcttttttacaggataagtttatgtatgtcacagatgactggattaattaagtgctaagttactactgccataaaaaactaataatacaatgtcactttatcagaatactagttttaaaagctgaatgttaataggggacactgtaaagtatcatcaaaacctgaatagcttcattgtgcacaagtgtggagttttgtatcctcttacctggtaaactgaagggattgtttggccatttcatttatcttatcattaattcacaagatagttagaaattctgcctcaagcaaagtaccacattttgaatgttttcttagattttgattgcaagtagatatcagcattttttaaatgaaaagctatattatcttctcccttcaaggcagcctaaggatgttctttcccagaatcactccaacccttcttgccagaattcataaaagtacaaaattggagaatagatgatatcttagaaataagctttttttttttttttttttttttttgagacggagtttcactcttgtcacccaggctgaagtgcaatggcgcaattagggttcactgcaacctctgcctcccgggttcaagcagttctcctgcctcagcctcctgagtagctgggattacaggcatccaccaccgtgcccagctaatttttgtatttttagtagagacggggttttgccatgttggacaggttgatctcaaactcctgacctcaggtgatctaccctcctcggcctcccagagtgttgggattacaggcatgagccaccatgccaggctgctaattctcctttttagtgagttagggaactgagcctcagaaaacttaaacgatttctcagaaaacactcaagtgataaagtggccacattggaaaggagtttttatcttctcattgtcaggccagtgttcattgcacaatatcatgctacctcttgaatctttaaaatattcaattggcaaatgtttttcaatgtgatttactcatgtcttaagtgtatgaggaaagttcaaagcaaaatagaaaggaataattcaaactgaattgtccataatcagcttccagtctttcatgctaatcagcttcttaagagactgaagtatggcatacctacaggggaattccttcgcaccatagcctgtatgaacagtgttccctggagttctccagtgctcagcttgagaccttgatacacgggccatgagccctgtcttccccaatggaaatttatttacacttaccttatccctatggacttagtctgattttattggctaggagtctaacagtcctgtgtggatatacagttttgcccatgacaacaaaggaatctatccgaaatatctttttttttataataaacttccaagatttgctgtcttccagcacttgagttaaagtactagatactgcattttgatgaagactaaccccatctcatattctaccctaaagagaactgaaaaacctataataagttgttctggagccaataaacacagcagctctgttagatgtcctctacagccaagcactttcaatgctaacttgaactgcatttccttcctcaaatgagagattgacataattcagtactgtgagtcacttgtataagaaacctttgatcactaaaaataatgtaaaaattgggtttagtagcctaatacacataacgttcttcttaaaaaggaaaatggatggatgcctgacaaccctccaaaagaaaaaagtgtaagatagccattaagatgatgacaatttttgaaatgaacattatgatatttatgaacaataaacaaatttccgtatggaatgaattatccaaaaagagtataacaaaatgaaatccttaaaaatccagagtttatattttttttataccctcacttgtttgcactaactttatagtggaccaaggctgttaccataggaagggacaaacttccttgtaggcaactcagtgttagacgatgattgtggttatgcttgcaaagtcttgtgcttatcttttttgtttttacttaaaaagctaatttttaaagattgtagggcttgtattttacttgaataattgatatcttcctgtgtaatgatttgtgagatgagaattaatatttgactagttagaattaattaaatggtaagggaacacagggtactcttaggttaaataatgtatgcaaatagagtctattttcaactaatatggccacaggagccttttgagattcattgatattaaacacaattaatgaaattttaaattgttaacagaattgagaacttgaacaacacttttagtactgcagcatttttgtgccctaaagtatgtaatgatttataaatgtgccatacatacactacaacataacatttgctttgttatgcattttatttctctggggacaccattgcactgcagtgcacacgtatttataaacatttgttatatttttggaaacttgctaatatttattaagtcatagacttttctggaggacttaaaaattcactaaaaatctgattatgtcttaaatgttcagtttatctttggtttattaaaataaaaaaaaaatctaagattaaacacagtagatatctctggaggcaattttccaaaactcaacattaaaatttgtggatgcatgagatgcaatccttcaaagaatgaatctgaaatatatttttaatatttacttaatatccactgaagatatctttatgcaagacaagagtcagccatcagacactgaaatatattatgatagattatgaagaattttctctgtagaattatattcttcctggaacctggtagagtagattagactcaaaggctttttcttccttttcttactcctgttttttccactcactcttcccaagagatttcctaaagcttcaagcttaataagcctaatagtgaaaaataactgaatttaatggtataatgaagttcttcatttccagacatctttaattgatcttaaagctcatttgagtctttgcccctgaacaaagacagacccattaaaatctaagaattctaaattttcacaactgtttgagcttcttttcattttgaaggatttggaatatatatgttttcataaaagtatcaagtgaaatatagttacatgggagctcaatcatgtgcagattgcattctgttatgttgactcaatatttaatttacaactatccttatttatattgacctcaagaactccattttatgcaatgcagaccactgagatatagctaacattctttcaaataattttccttttcttttataattcctctatagcaaatttttatgtataactgattatacatatccatatttatatttcattgattccaagacatcactttttcaatttaacatctctgaaattgtgacatttcttgcaactgttggcacttcagatgcagtgtttaaaattatgcttgaataaatattacactaatccaactttacctaaatgtttatgcatctaggcaaattttgttttcttataaagatttgagagcccatttatgacaaaatatgaaggcgaaatttaaggacaactgagtcacgcacaactcaacatggagcctaactgattatcagctcagatcccgcatatcttgagtttacaaaagctctttcaggtccccatttatactttacgtgagtgcgaatgatttcagcaaaccctaacttaactaacaagaatgggtaggtatgtctacgtttcattaacaaatttttattatttttattctattatatgagatccttttatattatcatctcacttttaaacaaaattaactggaaaaatattacatggaactgtcatagttaggttttgcagcatcttacatgtcttgtatcaatggcaggagaaaaatatgataaaaacaatcagtgctgtgaaaaacaactttcttctagagtcctcttactttttattcttctttatcatttgtgggtttttcccccttggctctgatcactttaacttcaagcttatgtaacgactgttataaaactgcatatttaaattatttgaattatatgaaataattgttcagctatctgggcagctgttaatgtaaacctgagagtaataacactactcttttatctacctggaatacttttctgcataaaatttatctttgtaagctaactctattaatcaggtttcttctagcctctgcaacctacttcagttagaattgtctaatactgctctattaatcaggtttctagcctctacaacctacttcagttaaaattgtctaatacagcaatatttaaaaaaaaaacactgcaattgtcaaggatggaaaatgtgtgatttgtgtaaacaatttttaccaactttacattttcctacagataaatgtgaaattttgataagaagtctacgcaatgacaagtatggtacataaattttattaagaatattgagtataaagtactttaattctaaattataagaaaatatacatttgcacatattaatatagaaattcattttgtgtatatttaacatagcttttaaactattttacattagctacttcattatggtttcttgaacttctgaaaaaaattagaaatgtattaaacttatcagtaacataaaaacttattttgtttcacctaacgaatactgcgtttgtaaaaataaatttaatatagaatatatttttaaattaaatatttgaatataaaatagctctaagaaagaagcaaattatcactgaacatatttcttattatttctggctttgaattatacgtaacttaaattgtcttaaatgatacagaatattggagaatatgatactttcacataatatactatgaacctgttcatataactctgattgactactaacttctgttttatgtatttattaaagagctgacactgtagtttgtggtgagatgtttatttttctaacagagcttataacagttaggacaaggcatttaattaatgcatcattctgtttagtagtaggtgttaatcaatatgaaattctctgttttaaaataaaaatgtaaaaatctaagaataaaaaaaaaaa

SEQ ID NO: 18 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 19 - DNA, 3147 bp, Homo sapiens
atggcttttccgccgcggcgacggctgcgcctcggtccccgcggcctcccgcttcttctctcgggactcctgctacctctgtgccgcgccttcaacctagacgtggacagtcctgccgagtactctggccccgagggaagttacttcggcttcgccgtggatttcttcgtgcccagcgcgtcttcccggatgtttcttctcgtgggagctcccaaagcaaacaccacccagcctgggattgtggaaggagggcaggtcctcaaatgtgactggtcttctacccgccggtgccagccaattgaatttgatgcaacaggcaatagagattatgccaaggatgatccattggaatttaagtcccatcagtggtttggagcatctgtgaggtcgaaacaggataaaattttggcctgtgccccattgtaccattggagaactgagatgaaacaggagcgagagcctgttggaacatgctttcttcaagatggaacaaagactgttgagtatgctccatgtagatcacaagatattgatgctgatggacagggattttgtcaaggaggattcagcattgattttactaaagctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatgatattgcaattgctgctccatatgggggtgaagataaaaaaggaattgtttatatcttcaatggaagatcaacaggcttgaacgcagtcccatctcaaatccttgaagggcagtgggctgctcgaagcatgccaccaagctttggctattcaatgaaaggagccacagatatagacaaaaatggatatccagacttaattgtaggagcttttggtgtagatcgagctatcttatacagggccagaccagttatcactgtaaatgctggtcttgaagtgtaccctagcattttaaatcaagacaataaaacctgctcactgcctggaacagctctcaaagtttcctgttttaatgttaggttctgcttaaaggcagatggcaaaggagtacttcccaggaaacttaatttccaggtggaacttcttttggataaactcaagcaaaagggagcaattcgacgagcactgtttctctacagcaggtccccaagtcactccaagaacatgactatttcaagggggggactgatgcagtgtgaggaattgatagcgtatctgcgggatgaatctgaatttagagacaaactcactccaattactatttttatggaatatcggttggattatagaacagctgctgatacaacaggcttgcaacccattcttaaccagttcacgcctgctaacattagtcgacaggctcacattctacttgactgtggtgaagacaatgtctgtaaacccaagctggaagtttctgtagatagtgatcaaaagaagatctatattggggatgacaaccctctgacattgattgttaaggctcagaatcaaggagaaggtgcctacgaagctgagctcatcgtttccattccactgcaggctgatttcatcggggttgtccgaaacaatgaagccttagcaagactttcctgtgcatttaagacagaaaaccaaactcgccaggtggtatgtgaccttggaaacccaatgaaggctggaactcaactcttagctggtcttcgtttcagtgtgcaccagcagtcagagatggatacttctgtgaaatttgacttacaaatccaaagctcaaatctatttgacaaagtaagcccagttgtatctcacaaagttgatcttgctgttttagctgcagttgagataagaggagtctcgagtcctgatcatgtctttcttccgattccaaactgggagcacaaggagaaccctgagactgaagaagatgttgggccagttgttcagcacatctatgagctgagaaacaatggtccaagttcattcagcaaggcaatgctccatcttcagtggccttacaaatataataataacactctgttgtatatccttcattatgatattgatggaccaatgaactgcacttcagatatggagatcaaccctttgagaattaagatctcatctttgcaaacaactgaaaagaatgacacggttgccgggcaaggtgagcgggaccatctcatcactaagcgggatcttgccctcagtgaaggagatattcacactttgggttgtggagttgctcagtgcttgaagattgtctgccaagttgggagattagacagaggaaagagtgcaatcttgtacgtaaagtcattactgtggactgagacttttatgaataaagaaaatcagaatcattcctattctctgaagtcgtctgcttcatttaatgtcatagagtttccttataagaatcttccaattgaggatatcaccaactccacattggttaccactaatgtcacctggggcattcagccagcgcccatgcctgtgcctgtgtgggtgatcattttagcagttctagcaggattgttgctactggctgttttggtatttgtaatgtacaggatgggcttttttaaacgggtccggccacctcaagaagaacaagaaagggagcagcttcaacctcatgaaaatggtgaaggaaactcagaaacttaa

SEQ ID NO: 20 - protein, 116 aa, Artificial Sequence
  VH region
EVQLVESGGGLVQPGGSLNLSCAVSGFVFSRYWMSWVRQAPGKGLEWIGEINPDSSTINYTSSLKDKFIISRDNAKNTLYLQMNKVRSEDTALYYCACLITTEDYWGQGTSVTVSS

SEQ ID NO: 21 - DNA, 348 bp, Artificial Sequence
  VH region
gaagtgcagctggtggagtctggaggtggcctggtgcagcctggaggatccctgaacctctcctgtgcagtctcaggattcgtttttagtagatactggatgagttgggtccggcaggctccagggaaagggctagaatggattggagaaattaatccagatagcagtacgataaactatacgtcatctctaaaggataaattcatcatctccagagacaacgccaaaaatacgttgtacctgcaaatgaacaaagtgagatctgaggacacagccctttattactgtgcatgtcttattactacggaggactactggggtcaaggaacctcagtcaccgtctcctca

SEQ ID NO: 22 - protein, 107 aa, Artificial Sequence
  VL region
EIVLTQSPSSMYASLGERVTIPCKASQDINSYLSWFQQKPGKSPKTLIYYANRLVDGVPSRFSGSGSGQDYSLTISSLEYEDMGIYYCLQYDEFPYTFGGGTKLEIK

SEQ ID NO: 23 - DNA, 321 bp, Artificial Sequence
  VL region
gaaattgtgctgactcagtctccatcttccatgtatgcatctctaggagagagagtcactatcccttgcaaggcgagtcaggacattaatagctatttaagctggttccagcagaaaccagggaaatctcctaagaccctgatctattatgcaaacagattggtagatggggtcccatcaaggttcagtggcagtggatctgggcaagattattctctcaccatcagcagcctggagtatgaagatatgggaatttattattgtctacagtatgatgagtttccgtacacgttcggaggaggcaccaagctggaaatcaaa

SEQ ID NO: 24 - protein, 116 aa, Artificial Sequence
  VH region
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCASLITTEDYWGQGTTVTVSS

SEQ ID NO: 25 - protein, 107 aa, Artificial Sequence
  VL region
EIVLTQSPSSLSLSPGERVTITCKASQDINSYLSWYQQKPGKAPKLLIYYANRLVDGVPARFSGSGSGQDYTLTISSLEPEDFAVYYCLQYDEFPYTFGGGTKLEIK

SEQ ID NO: 26 - protein, 116 aa, Artificial Sequence
  P2-23
EVQLVESGGGLVQPGGSLRLSCAVSGFVFSRYWVSWVRQAPGKGLEWIGEINPDSSTINYTSSLKDRFTISRDNAKNSLYLQMNSLRAEDTAVYYCAILITTEDYWGQGTTVTVSS

SEQ ID NO: 27 - protein, 116 aa, Artificial Sequence
  P2-33
EVQLVESGGGLVQPGGSLRLSCAVSGFVFSRYWISWVRQAPGKGLEWIGEINPDSSTINYTSSLKDRFTISRDNAKNSLYLQMNSLRAEDTAVYYCAILITTEDYWGQGTTVTVSS

SEQ ID NO: 28 - protein, 116 aa, Artificial Sequence
  P2-25
EVQLVESGGGLVQPGGSLRLSCAVSGFVFSRSWMSWVRQAPGKGLEWIGEINPDSSTINYTSSLKDRFTISRDNAKNSLYLQMNSLRAEDTAVYYCAILITTEDYWGQGTTVTVSS

SEQ ID NO: 29 - protein, 116 aa, Artificial Sequence
  P1-21
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCAILITDEDYWGQGTTVTVSS

SEQ ID NO: 30 - protein, 116 aa, Artificial Sequence
  P1-35
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCAIFITTEDYWGQGTTVTVSS

SEQ ID NO: 31 - protein, 107 aa, Artificial Sequence; identical to SEQ ID NO: 8
  C941

SEQ ID NO: 32 - protein, 107 aa, Artificial Sequence
  P1-42
DIQLTQSPSSLSASVGDRVTITCKASQDINKYLSWFQQKPGKAPKSLIYYANRLVDGVPSRFSGSGSGTDFTLTISSLQPEDFATYYCLQYDEFPYTFGGGTKVEIK

SEQ ID NO: 33 - protein, 107 aa, Artificial Sequence
  P2-16
DIQLTQSPSSLSASVGDRVTITCKASQDINSYLSWFQQKPGKAPKSLIYYANRLVDGVPSRFSGSGSGTDFTLTISSLQPEDFATYYCGQYDEFPYTFGGGTKVEIK

SEQ ID NO: 34 - protein, 107 aa, Artificial Sequence
  P2-19
DIQLTQSPSSLSASVGDRVTITCKASQDINSYLSWFQQKPGKAPKSLIYYANRLVDGVPSRFSGSGSGTDFTLTISSLQPEDFATYYCLQYDVFPYTFGGGTKVEIK

SEQ ID NO: 35 - protein, 107 aa, Artificial Sequence
  P2-36
DIQLTQSPSSLSASVGDRVTITCKASQDINSYLSWFQQKPGKAPKSLIYYANRLVDGVPSRFSGSGSGTDFTLTISSLQPEDFATYYCLQYDEFVYTFGGGTKVEIK

SEQ ID NO: 36 - protein, 107 aa, Artificial Sequence
  P2-14
DIQLTQSPSSLSASVGDRVTITCKASQDINSYLSWFQQKPGKAPKSLIYYANRLVDGVPSRFSGSGSGTDFTLTISSLQPEDFATYYCLQADEFPYTFGGGTKVEIK

(19) United States
(12) Patent Application Publication
(10) Pub. No.: US 2023/0112035 A1
(43) Pub. Date: Apr. 13, 2023

(54) ANTI-avB8 INTEGRIN ANTIBODIES FOR USE IN TREATING KIDNEY DISEASE

(71) Applicant: MEDIMMUNE LIMITED, Cambridge (GB)

(72) Inventors: David James BAKER, Cambridge (GB); Stephanie Claire HEASMAN, Cambridge (GB); Maria Marcela HERRERA, Cambridge (GB); Elena LIARTE MARIN, Cambridge (GB); Carol Patricia MORENO-QUINN, Cambridge (GB); Lynne Anne MURRAY, Cambridge (GB); Ping TSUI, Gaithersburg, MD (US); Yanli WU, Gaithersburg, MD (US)

(21) Appl. No.: 17/759,388

(22) PCT Filed: Jan. 26, 2021
(86) PCT No.: PCT/EP2021/051753

§ 371 Date: Jul. 25, 2022

Related U.S. Application Data
(60) Provisional application No. 62/966,258, filed on Jan. 27, 2020.

(51) Int. Cl. C07K 16/28; A61P 13/12; G01N 33/68

(52) U.S. Cl. CPC C07K 16/2839; A61P 13/12; G01N 33/6893; C07K 2317/24; C07K 2317/92; C07K 2317/565; C07K 2317/56; A61K 2039/505

(57) ABSTRACT

Provided are methods and compositions for treating kidney disease, such as chronic kidney disease (CKD), in which the methods and compositions comprise antibodies or an antigen binding fragment thereof that specifically and selectively bind to human αvβ8 integrin, which was discovered, as described, to be highly expressed on kidney cells and tissue, and, in particular, diseased or fibrotic kidney tissue. The disclosed anti-αvβ8 integrin antibodies bind to human αvβ8 integrin in the kidney and block the activation of TGF-β from its latent form in kidney tissue. The anti-αvβ8 antibodies in the disclosed methods reduce, attenuate, or abrogate kidney fibrosis, which is associated with the activities of αvβ8 integrin and TGF-β in kidney tissue. The disclosed antibodies and methods effectively treat kidney disease, in particular, fibrosis associated with kidney disease, such as CKD, in individuals in need thereof.

### BACKGROUND

Kidney disease generally refers to a condition in which an individual's kidneys are damaged and cannot function properly to filter waste products and excess water from the blood or to help control blood pressure. The kidneys function to release hormones that regulate blood pressure, produce vitamin D and control the production of red blood cells. Damage to the kidneys can cause wastes to accumulate in the body and can also cause or increase an individual's risk of other health problems, such as heart disease, heart attack, or stroke. Major risk factors for kidney disease include diabetes, high blood pressure and family history of kidney failure. Kidney disease can include acute kidney injury (AKI) which involves a sudden and sometimes temporary loss of kidney function and chronic kidney disease (CKD), which refers to any condition that causes reduced kidney function over a prolonged period of time. CKD may develop over many years and may lead to end-stage kidney (renal) disease (ESRD).

Kidney disease is the ninth leading cause of death in the United States, according to the American Kidney Fund's 2015 kidney disease statistics. About 10% to 14% of the general adult population in the U.S. has CKD, which is more prevalent in women. However, men with CKD are 50% more likely than women to have a condition of CKD develop into kidney failure or ESRD. In addition, certain racial and ethnic groups are at greater risk of kidney failure than others. For example, compared to Caucasians, ESRD prevalence is about 3.7 times greater in African Americans, 1.4 times greater in Native Americans, and 1.5 times greater in Asian Americans. Compared to non-Hispanics, Hispanics are nearly 1.5 times as likely to have ESRD.

Renal disease progression often leads to the complete destruction of functional kidney tissue, which ultimately can cause affected individuals to require life-long dialysis or to undergo renal allograft transplantation. Renal disease progression is characterized by fibrosis, which contributes to the destruction of the glomerulus (glomerulosclerosis) and renal tubules (tubulo-interstitital fibrosis). A key factor in the progression of renal fibrosis is the cytokine TGF-β, its interactive molecules and receptors, and its downstream cellular signaling cascades that activate pathophysiological cellular mechanisms leading to renal fibrosis and renal function decline. TGF-β expression in the kidney and TGF-β excretion correlate with glomerular filtration rate (GFR) decline and protein leakage (proteinuria). Renal disease progression often leads to the complete destruction of functional kidney tissue, which causes affected individuals to require life-long dialysis or to undergo renal allograft transplantation. At a molecular level, the connections and interplay among TGF-β, which is involved in many pathologies including neoplastic diseases and inflammation, and its receptors and signaling mechanisms, still remain elusive.

For the treatment of kidney disease, there is a significant need for tissue and disease-specific modulators of TGF-β activity that do not result in undesirable off-target effects and that do not adversely affect the other physiological contributions of this cytokine to normal cellular functions. The methods and the reagents as described herein provide advantageous and beneficial treatment for kidney disease, as well as specifically targeted antibodies as treatment components that have direct effects on blocking, neutralizing, modulating, and/or inhibiting a critical integrin target that has been discovered to play a significant role in kidney cell and tissue pathologies and in the mechanism of kidney disease.

### SUMMARY

As described below, the present disclosure features therapeutic methods of treating a subject, particularly a mammalian subject, and more particularly, a human subject, who has kidney disease, in particular, chronic kidney disease (CKD) and/or symptoms thereof with an antibody, or an antigen-binding fragment thereof, that specifically binds to αvβ8 integrin, i.e., an anti-αvβ8 integrin antibody. Based on the findings described herein, high levels of expression of αvβ8 integrin on kidney cells and tissue, and especially diseased kidney cells and tissue, such as in subjects having kidney disease, e.g., CKD, allow an anti-αvβ8 integrin antibody or an antigen binding fragment thereof to specifically target the αvβ8 integrin expressed on diseased kidney tissues of subjects afflicted with kidney disease, such as CKD. The described anti-αvβ8 integrin antibodies do not cross-react with other integrin receptor isoforms, such as αvβ1, αvβ3, αvβ5, or αvβ6. In embodiments, the anti-αvβ8 integrin antibodies are isolated and purified antibodies. In a particular embodiment, the anti-αvβ8 integrin antibody is a humanized antibody. In another particular embodiment, the anti-αvβ8 integrin antibody is humanized and affinity optimized to have improved structural, binding and/or functional properties, such as improved specificity, affinity and/or stability.

The anti-αvβ8 integrin antibodies and methods described herein were developed based on the discovery that kidney cells and tissue, particularly epithelial tissue of the kidney, express high levels of αvβ8 integrin, which is a receptor for the latent form of the TGF-β cytokine (LAP-TGF-β). In particular, the elevated levels of αvβ8 integrin expressed on kidney epithelium were discovered, as evidenced by the experimental embodiments described herein, to be highly correlated with fibrosis and/or with the severity of fibrosis and fibrotic disease in kidney tissue in human subjects and in animal models having kidney disease. In particular, compared with normal kidney cells and tissue, the glomerular and tubule cells in the epithelial tissue of the kidney from individuals having kidney disease, such as diabetic nephropathy (DN), showed high levels of αvβ8 integrin expression, particularly, in the podocytes and tubules (such as the proximal and distal cortical tubules) of diseased kidney, as exemplified herein. In addition to diabetic nephropathy, other nonlimiting types of kidney disease in which fibrosis of kidney tissue produces debilitating damage and dysfunction in renal activity and function include chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like.

For the treatment of kidney disease and CKD, the αvβ8 integrin, which binds to TGF-β in its latent form, has been shown by the practice of the methods involving the antibody reagents described herein to be a highly effective and useful target for reducing and attenuating fibrosis and tissue damage that are hallmarks of kidney disease such as CKD. As described and exemplified herein, αvβ8 integrin plays a direct, significant, and previously unrecognized, role in kidney fibrosis, in view of the high level of expression of αvβ8 integrin in kidney cells and tissue, particularly kidney epithelial cells and tissue, e.g., in the glomeruli podocytes and tubule cells of the kidney, and in the modulation of the activity of TGF-β, which is involved in and compounds the damaging effects of, fibrosis in kidney tissue in subjects with kidney disease. The anti-αvβ8 integrin antibodies used in the described methods allow for the specific and selective modulation of the activities of both αvβ8 integrin and its TGF-β ligand in kidney cells and tissue, thereby effecting the reduction, abrogation, attenuation, decrease, and/or inhibition of fibrosis in kidney tissue caused by the binding of αvβ8 integrin to its ligand, latent TGF-β, which then enables the activation/release of active TGF-β and unleashes the deleterious effects of active TGF-β in causing fibrosis of kidney tissue.

One way that TGF-β can be activated in kidney tissue is by its association, as latency associated peptide (LAP) TGF-β, with αvβ8 integrin expressed in the membrane of kidney cells. As shown herein, the expression of αvβ8 integrin was found to be highly elevated in kidney tissue of subjects having kidney disease and accompanying kidney fibrosis. Sustained or prolonged TGF-β activation in the kidney causes fibrosis in kidney tissue. The targeting of αvβ8 integrin, particularly in kidney tissue expressing high levels of αvβ8 integrin, by the anti-αvβ8 integrin antibodies described herein, was determined to be an effective therapeutic treatment for kidney disease, for example, CKD, while avoiding a number of the potential systemic effects of indiscriminate TGF-β targeting and suppression in other, non-kidney tissues of the body. The UUO model is a model of fibrosis, and inhibition of fibrosis and TGF-β activation, as demonstrated by the anti-αvβ8 integrin antibodies described herein, will lead to a reduction and/or inhibition of CKD progression. As described herein, a high affinity anti-αvβ8 integrin antibody that specifically binds to αvβ8 integrin that is highly expressed in diseased and/or fibrotic kidney tissue also selectively reduces, abrogates, attenuates, decreases, neutralizes and/or inhibits or otherwise prevents the interaction of αvβ8 integrin and latent TGF-β at the membrane of kidney cells and tissue. Other tissues and organs not expressing αvβ8 integrin will not be affected. The specific binding of the anti-αvβ8 antibody to kidney cell-expressed αvβ8 integrin thereby blocks the activation of TGF-β in the diseased and/or fibrotic kidney so as to treat the kidney disease, such as CKD, and associated fibrosis, with minimal to no significant adverse effects on the activity of TGF-β in non-kidney cells and tissues.

Advantageously, the anti-αvβ8 integrin antibodies specifically mitigate the effects of the αvβ8 integrin and latent TGF-β interaction on the development and progression of fibrosis in kidney tissue and kidney disease, while sparing much of the contribution of TGF-β activity to normal cellular functions. In embodiments, the methods described herein further afford therapeutic treatment benefit for the protection of functional kidney epithelium in an individual having kidney disease involving fibrosis, such as CKD and other kidney diseases, such as diabetic nephropathy (DN)-associated kidney disease, acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like.

In an aspect, a method of treating kidney fibrosis in a subject having kidney disease is provided, in which the method comprises administering to the subject an effective amount of an anti-αvβ8 integrin antibody or an antigen-binding fragment thereof.

In another aspect, a method of reducing or attenuating kidney fibrosis in a subject having kidney disease is provided, in which the method comprises administering to a subject in need thereof an effective amount of an anti-αvβ8 integrin antibody or an antigen-binding fragment thereof, thereby reducing or attenuating fibrosis in the kidney.

In another aspect, a method of abrogating the activity of αvβ8 integrin associated with kidney fibrosis is provided, in which the method comprises administering to a subject in need thereof an effective amount of an anti-αvβ8 integrin antibody, or an antigen-binding fragment thereof, and blocks binding of αvβ8 integrin to latent TGF-β, thereby abrogating the activity of αvβ8 integrin associated with kidney fibrosis. In an embodiment of the method, the subject has kidney disease.

In yet another aspect, a method of treating kidney fibrosis by blocking the activation of TGF-β from its latent form in kidney cells and tissue is provided, in which the method comprises administering to a subject in need thereof an effective amount of an anti-αvβ8 integrin antibody or an antigen-binding fragment thereof and blocks αvβ8 integrin from binding to the latent form of TGF-β to produce active TGF-β, thereby treating the kidney fibrosis. In an embodiment of the method, the subject has kidney disease.

A method of treating kidney damage characterized by an increase in plasma creatinine and/or urinary protein excretion levels is provided, in which the method comprises administering to a subject in need thereof an effective amount of an anti-αvβ8 integrin antibody or an antigen binding fragment thereof, wherein administration of the anti-αvβ8 integrin antibody or an antigen binding fragment thereof abrogates the plasma creatinine and/or urinary protein excretion levels in the subject, thereby treating kidney damage.

In an embodiment, the anti-αvβ8 integrin antibody or an antigen binding fragment thereof decreases αvβ8-mediated TGF-β activation in the subject's kidney tissue.

In embodiments of any aspect of the above methods delineated herein, the kidney disease is selected from diabetic nephropathy (DN), chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like. In a particular embodiment, the kidney disease is CKD. In a particular embodiment, the kidney disease is diabetic nephropathy.

In an embodiment of any aspect of the above methods delineated herein, the anti-αvβ8 integrin antibody) or an antigen binding fragment thereof binds to αvβ8 integrin expressed on kidney cells and/or tissue and blocks the activation of TGF-β from its latent form.

Provided as another aspect as described herein is a method of detecting kidney fibrosis in kidney tissue, in which the method comprises contacting kidney tissue with an effective amount of a detectably labeled anti-αvβ8 integrin antibody or an antigen binding fragment thereof, detecting the binding of the anti-αvβ8 integrin antibody to αvβ8 integrin in the kidney tissue.

In an embodiment of any aspect of the above methods delineated herein, the anti-αvβ8 integrin antibody, or an antigen-binding fragment thereof, which specifically binds to αvβ8 integrin, comprises:

(a) a heavy chain variable region complementarity determining region 1 (CDR1) comprising the amino acid sequence:

(SEQ ID NO: 1)
RYWMS;

(b) a heavy chain variable region complementarity determining region 2 (CDR2) comprising the amino acid sequence:

(SEQ ID NO: 2)
EINPDSSTINYTSSL;

and

(c) a heavy chain variable region complementarity determining region 3 (CDR3) CDR3 comprising the amino acid sequence:

(SEQ ID NO: 3)
LITTEDY;

and

(d) a light chain variable region CDR1 comprising the amino acid sequence:

(SEQ ID NO: 4)
KASQDINSYLS;

(e) a light chain variable region CDR2 comprising the amino acid sequence:

(SEQ ID NO: 5)
YANRLVD;

and

(f) a light chain variable region CDR3 comprising the amino acid sequence:

(SEQ ID NO: 6)
LQYDEFPYT.

In another embodiment of any aspect of the above methods delineated herein, the anti-αvβ8 integrin antibody, or an antigen-binding fragment thereof, which specifically binds to αvβ8 integrin, comprises a heavy chain variable region (V_{H}) amino acid sequence:

(SEQ ID NO: 7)
EVQLVESGGGLVQPGGSLRLSCAVSGFVFSRYWMSWVRQAPGKGLEWIGEI
NPDSSTINYTSSLKDRFTISRDNAKNSLYLQMNSLRAEDTAVYYCAILITT
EDYWGQGTTVTVSS;

and
a light chain variable region (V_{L}) amino acid sequence:

(SEQ ID NO: 8)
DIQLTQSPSSLSASVGDRVTITCKASQDINSYLSWFQQKPGKAPKSLIYYA
NRLVDGVPSRFSGSGSGTDFTLTISSLQPEDFATYYCLQYDEFPYTFGGGT
KVEIK.

In another embodiment of any aspect of the above methods delineated herein, the anti-αvβ8 integrin antibody, or an antigen-binding fragment thereof, which specifically binds to αvβ8 integrin, comprises:

(a) a heavy chain variable region CDR1 comprising the amino acid sequence:

(SEQ ID NO: 9)
RSWIS;

(b) a heavy chain variable region CDR2 comprising the amino acid sequence:

(SEQ ID NO: 2)
EINPDSSTINYTSSL;

and

(c) a heavy chain variable region CDR3 comprising the amino acid sequence:

(SEQ ID NO: 3)
LITTEDY;

and

(d) a light chain variable region CDR1 comprising the amino acid sequence:

(SEQ ID NO: 10)
KASQDINKYLS;

(e) a light chain variable region CDR2 comprising the amino acid sequence:

(SEQ ID NO: 5)
YANRLVD;

and

(f) a light chain variable region CDR3 comprising the amino acid sequence:

(SEQ ID NO: 11)
LQYDVFPYT.

In another embodiment of any aspect of the above methods delineated herein, the anti-αvβ8 integrin antibody (called “B5-15” herein), or an antigen-binding fragment thereof, which specifically binds to αvβ8 integrin, comprises a heavy chain variable region (V_{H}) amino acid sequence:

(SEQ ID NO: 12)
EVQLVESGGGLVQPGGSLRLSCAVSGFVFSRSWISWVRQAPGKGLEWIGE
INPDSSTINYTSSLKDRFTISRDNAKNSLYLQMNSLRAEDTAVYYCAILI
TTEDYWGQGTTVTVSS

and
a light chain variable region (V_{L}) amino acid sequence:

(SEQ ID NO: 13)
DIQLTQSPSSLSASVGDRVTITCKASQDINKYLSWFQQKPGKAPKSLIYY
ANRLVDGVPSRFSGSGSGTDFTLTISSLQPEDFATYYCLQYDVFPYTFGG
GTKVEIK.

In an embodiment of any aspect of the above treatment methods delineated herein, the anti-αvβ8 integrin antibody, or an antigen binding fragment thereof, which specifically binds to αvβ8 integrin, is administered to the subject in combination with an adjunct therapeutic agent or treatment for kidney disease. In embodiments, the anti-αvβ8 integrin antibody, or an antigen binding fragment thereof, which specifically binds to αvβ8 integrin, is administered to the subject prior to, at the same time as, or after the administration of the adjunct therapeutic agent or treatment.

In an embodiment of any aspect of the above treatment methods delineated herein, the anti-αvβ8 integrin antibody, or an antigen binding fragment thereof, binds to αvβ8 integrin having increased expression on fibrotic kidney cells and tissue and attenuates or abrogates fibrosis associated with increased expression of αvβ8 integrin in podocytes and interstitial tubule cells in kidney tissue of the subject with kidney disease, such as CKD.

Provided in another aspect as described herein is an anti-αvβ8 integrin antibody or an antigen binding fragment thereof, wherein the antibody or an antigen binding fragment thereof comprises:

(a) a heavy chain variable region CDR1 comprising the amino acid sequence:

(SEQ ID NO: 1)
RYWMS;

(b) a heavy chain variable region CDR2 comprising the amino acid sequence:

(SEQ ID NO: 2)
EINPDSSTINYTSSL;

and

(c) a heavy chain variable region CDR3 comprising the amino acid sequence:

(SEQ ID NO: 3)
LITTEDY;

and

(d) a light chain variable region CDR1 comprising the amino acid sequence:

(SEQ ID NO: 4)
KASQDINSYLS;

(e) a light chain variable region CDR2 comprising the amino acid sequence:

(SEQ ID NO: 5)
YANRLVD;

and

(f) a light chain variable region CDR3 comprising the amino acid sequence:

(SEQ ID NO: 6)
LQYDEFPYT

Provided in another aspect as described herein is an anti-αvβ8 integrin antibody, or an antigen binding fragment thereof, wherein the antibody or an antigen binding fragment thereof comprises:

a heavy chain variable region (V_{H}) amino acid sequence:

(SEQ ID NO: 7)
EVQLVESGGGLVQPGGSLRLSCAVSGFVFSRYWMSWVRQAPGKGLEWIGE
INPDSSTINYTSSLKDRFTISRDNAKNSLYLQMNSLRAEDTAVYYCAILI
TTEDYWGQGTTVTVSS;

and

a light chain variable region (V_{L}) amino acid sequence:

(SEQ ID NO: 8)
DIQLTQSPSSLSASVGDRVTITCKASQDINSYLSWFQQKPGKAPKSLIYY
ANRLVDGVPSRFSGSGSGTDFTLTISSLQPEDFATYYCLQYDEFPYTFGG
GTKVEIK.

In an embodiment, the above antibody, or an antigen binding fragment thereof, binds to αvβ8 integrin having increased expression on fibrotic kidney cells and tissue in a subject having kidney disease, such as CKD. In an embodiment, the anti-αvβ8 integrin antibody, or an antigen-binding fragment thereof, specifically binds to αvβ8 integrin having increased expression on fibrotic kidney cells and tissue and blocks binding of αvβ8 integrin to latent TGF-β, thereby abrogating the activity of αvβ8 integrin associated with kidney fibrosis. In an embodiment, the anti-αvβ8 integrin antibody, or an antigen binding fragment thereof, attenuates or abrogates fibrosis associated with increased expression of αvβ8 integrin in podocytes and interstitial tubule cells in kidney tissue of the subject with kidney disease.

Provided in another aspect as described herein is an anti-αvβ8 integrin antibody, or an antigen binding fragment thereof, wherein the antibody or an antigen binding fragment thereof comprises:

(a) a heavy chain variable region CDR1 comprising the amino acid sequence:

(SEQ ID NO: 9)
RSWIS;

(b) a heavy chain variable region CDR2 comprising the amino acid sequence:

(SEQ ID NO: 2)
EINPDSSTINYTSSL;

and

(c) a heavy chain variable region CDR3 comprising the amino acid sequence:

(SEQ ID NO: 3)
LITTEDY;

and

(d) a light chain variable region CDR1 comprising the amino acid sequence:

(SEQ ID NO: 10)
KASQDINKYLS;

(e) a light chain variable region CDR2 comprising the amino acid sequence:

(SEQ ID NO: 5)
YANRLVD;

and

(f) a light chain variable region CDR3 comprising the amino acid sequence:

(SEQ ID NO: 11)
LQYDVFPYT

Provided in another aspect as described herein is an anti-αvβ8 integrin antibody, or an antigen binding fragment thereof, wherein the antibody or an antigen binding fragment thereof comprises a heavy chain variable region (V_{H}) amino acid sequence:

(SEQ ID NO: 12)
EVQLVESGGGLVQPGGSLRLSCAVSGFVFSRSWISWVRQAPGKGLEWIGE
INPDSSTINYTSSLKDRFTISRDNAKNSLYLQMNSLRAEDTAVYYCAILI
TTEDYWGQGTTVTVSS

and
a light chain variable region (V_{L}) amino acid sequence:

(SEQ ID NO: 13)
DIQLTQSPSSLSASVGDRVTITCKASQDINKYLSWFQQKPGKAPKSLIYY
ANRLVDGVPSRFSGSGSGTDFTLTISSLQPEDFATYYCLQYDVFPYTFGG
GTKVEIK.

In an embodiment, the above antibody, or an antigen binding fragment thereof, binds to αvβ8 integrin having increased expression on fibrotic kidney cells and tissue in a subject having kidney disease, such as CKD. In an embodiment, the anti-αvβ8 integrin antibody, or an antigen-binding fragment thereof, specifically binds to αvβ8 integrin having increased expression on fibrotic kidney cells and tissue and blocks binding of αvβ8 integrin to latent TGF-β, thereby abrogating the activity of αvβ8 integrin associated with kidney fibrosis. In an embodiment, the anti-αvβ8 integrin antibody, or an antigen binding fragment thereof, attenuates or abrogates fibrosis associated with increased expression of αvβ8 integrin in podocytes and interstitial tubule cells in kidney tissue of the subject with kidney disease.

In an embodiment of any of the above aspects, the anti-αvβ8 integrin antibody, or an antigen binding fragment thereof, is of the IgG class. In a particular embodiment, the antibody or an antigen binding fragment thereof is of the IgG1 isotype.

In another aspect, an anti-αvβ8 integrin antibody, or an antigen binding fragment thereof, is provided that competes for binding to αvβ8 integrin with the antibody or an antigen binding fragment thereof of any of the anti-αvβ8 integrin antibodies as described in the above aspects. In an embodiment, the anti-αvβ8 integrin antibody or an antigen binding fragment thereof is an IgG antibody. In an embodiment, the anti-αvβ8 integrin antibody or an antigen binding fragment thereof is an IgG1 antibody.

In another aspect, a polynucleotide encoding the anti-αvβ8 integrin antibody, or an antigen binding fragment thereof, as described herein is provided. In an embodiment, the polynucleotide sequence encoding the V_{H} region of the antibody comprises the following nucleic acid sequence:

(SEQ ID NO: 14)
gaggtgcagctggtggaaagcggcggaggactggtgcagcctggcggcag
cctgagactgagctgcgccgtgtccggcttcgtgttcagccggagctgga
tcagctgggtccgccaggccccagggaagggcctggaatggatcggcgag
atcaaccccgacagcagcaccatcaactacaccagcagcctgaaggaccg
gttcaccatcagccgggacaacgccaagaacagcctgtacctgcagatga
acagcctgcgggccgaggacaccgccgtgtactactgcgccatcctcatc
accaccgaggactactggggccagggcaccaccgtgaccgtgtcctct;

and the polynucleotide sequence encoding the V_{L} region of the antibody comprises the following nucleic acid sequence:

(SEQ ID NO: 15)
gacatccagctgacccagagccccagcagcctgagcgccagcgtgggcga
cagagtgaccatcacatgcaaggccagccaggacatcaacaagtacctga
gctggttccagcagaagcccggcaaggcccccaagagcctgatctactac
gccaaccggctggtggacggcgtgcccagcagattttctggcagcggcag
cggcaccgacttcaccctgaccatcagcagcctgcagcccgaggacttcg
ccacctactactgcctgcagtacgacgtgttcccctacaccttcggcgga
ggcaccaaggtggaaatcaag.

In another aspect, an expression vector which comprises a polynucleotide as described above is provided. In embodiments, the expression vector is a prokaryotic, eukaryotic, or mammalian expression vector.

In another aspect, a cell comprising the expression vector as described above is provided. In embodiments, the cell is a prokaryotic, a eukaryotic, or a mammalian host cell.

In another aspect, a pharmaceutical composition comprising the anti-αvβ8 integrin antibody or an antigen-binding fragment thereof as delineated above, and a pharmaceutically acceptable carrier, excipient, or diluent, is provided.

In another aspect, a pharmaceutical composition comprising the polynucleotide as delineated above, and a pharmaceutically acceptable carrier, excipient, or diluent, is provided.

In another aspect, a kit comprising the anti-αvβ8 integrin antibody, or an antigen binding fragment thereof, as described herein, or a pharmaceutical composition comprising the anti-αvβ8 integrin antibody or the antigen binding fragment thereof, is provided.

Other features and advantages of the present disclosure will be apparent from the detailed description, and the claims.

### Definitions

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “agent” refers to a protein, polypeptide, peptide (or fragment or portion thereof), nucleic acid molecule, small compound, drug, or medicine. The agent may be antagonistic and block or inhibit the activity of another molecule, such as a cognate ligand.

The term “antibody,” as used in this disclosure, refers to an immunoglobulin or a fragment, portion, or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments (or portions) such as Fab, F(ab′)2, Fv, scFv, Fd, dAb, and other antibody fragments (or portions) that retain antigen-binding function or epitope-binding function, i.e., the ability to bind a polypeptide specifically. Typically, such fragments (or portions) comprise an antigen-binding domain.

By way of example, an immunoglobulin (antibody) comprises a tetrameric structural unit. Each tetramer contains two identical pairs of polypeptide chains, each pair having one “light” (L) chain (about 25 kD) and one “heavy” (H) chain (about 50-70 kD). The amino (N)-terminus of each polypeptide chain defines a variable (V) region of about 100 to 110 or more amino acids that are primarily responsible for antigen recognition and binding. The terms variable light chain region (V_{L}) and variable heavy chain region (V_{H}) refer to the variable regions of the light and heavy chains, respectively, of the immunoglobulin molecule (antibody). A variable region or “V region” refers to an antibody variable region domain comprising component segments, namely, a Framework 1 (F1), CDR1, Framework 2 (F2), CDR2, Framework 3 (F3), CDR3, and Framework 4 (F4), which result from the genetic rearrangement of the heavy chain and light chain V region genes during B cell differentiation.

The V_{H} and V_{L} regions of immunoglobulin (antibody) molecules comprise three complementarity determining regions (CDRs), which are three hypervariable regions that are situated within the V_{H} and V_{L} framework regions. The CDRs are primarily responsible for binding to an epitope of an antigen. The CDRs of the antibody V_{H} and V_{L} regions are typically referred to as CDR1, CDR2, and CDR3, numbered sequentially starting from the N-terminus of the variable region segments. The amino acid sequences of the framework regions of different heavy and light antibody chains are relatively conserved within a species. The framework regions (FW1-FW4) of the constituent heavy and light chain V regions of an antibody provide structural positioning and alignment of the CDRs in three-dimensional space. Characterization (and numbering) of the amino acid sequences of the CDRs and framework regions in antibody molecules can be determined as reported by, for example, Kabat, Chothia, International ImMunoGeneTics database (IMGT), and AbM (e.g., Chothia & Lesk, 1987, J. Mol. Biol., 196:901-917; Chothia et al., 1989, Nature, 342:877-883; Chothia et al., 1992, J. Mol. Biol., 227:799-817; Al-Lazikani et al., 1997, J. Mol. Biol., 273(4):927-948). Definitions of antigen combining sites are reported in Ruiz et al., 2000, Nucleic Acids Res., 28:219-221 and Lefranc, 2001, Nucleic Acids Res., 29(1):207-209; MacCallum et al., 1996, J. Mol. Biol., 262:732-745; Martin et al, 1989, Proc. Natl Acad. Sci. USA, 86:9268-9272; Martin, et al, 1991, Methods Enzymol., 203:121-153; Pedersen et al, 1992, Immunomethods, 1:126-136; and Rees et al, 1996, In: Sternberg M. J. E. (ed.), Protein Structure Prediction. Oxford University Press, Oxford, England, pp. 141-172.

A “chimeric antibody” is an antibody molecule in which the constant region, or a fragment thereof, is altered, replaced or exchanged so that the antigen binding site (variable region, CDR, or fragment thereof) is linked to a constant region of an antibody molecule of a different or altered class and/or species, or to an entirely different molecule that confers new properties or effector function to the chimeric antibody (e.g., an enzyme, toxin, hormone, growth factor, drug, etc.). Alternatively, a chimeric antibody may comprise a variable region, or a fragment thereof, that is altered, replaced, or exchanged with a variable region having a different or altered antigen specificity (e.g., one or more CDRs and framework regions from different species).

The terms “antigen-binding portion,” “antigen-binding domain,” “antigen-binding fragment,” “binding fragment,” or “binding portion” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as a “binding site”, an “epitope” or an “antigenic determinant.” In particular embodiments, an antigen-binding domain comprises an antibody light chain variable region (V_{L}) and an antibody heavy chain variable region (V_{H}); however, it does not necessarily have to comprise both. For example, a so-called Fd antibody fragment consists only of a V_{H} domain, but still retains some antigen-binding function of the intact antibody. The binding site or epitope of an antibody produced against a given antigen can be determined using methods known in the art. For example, a competition assay (e.g., a competitive enzyme linked immunosorbent assay (ELISA)) can be carried out using an antibody with a known epitope. If a test antibody competes for binding to a given antigen, then the antibody likely shares at least part of the same epitope. The epitope can also be localized using domain swapping or selective mutagenesis of the antigen. That is, each region or each amino acid of the antigen can be “swapped” out, or substituted, with amino acids or components that are known not to interact with the test antibody. If substitution of a given region or amino acid reduces binding of the test antibody to the substituted antigen compared with the non-substituted antigen, then that region or amino acid is likely to be the epitope, to be within the epitope, or to be at least a part of the epitope.

Binding fragments (or portions) of an antibody are produced by recombinant DNA techniques, or by enzymatic or chemical cleavage of intact antibodies. Binding fragments or portions include Fab, Fab′, F(ab′)₂, FIT and single-chain antibodies. An antibody other than a “bispecific” or “bifunctional” antibody is understood to have each of its binding sites identical. Digestion of antibodies with the enzyme papain results in two identical antigen-binding fragments, known also as “Fab” fragments, and a “Fc” fragment, having no antigen-binding activity but having the ability to crystallize. Digestion of antibodies with the enzyme pepsin yields an F(ab′)₂ fragment in which the two arms of the antibody molecule remain linked and comprise two-antigen binding sites. The F(ab′)₂ fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies disclosed herein comprise without limitation whole native antibodies, bispecific antibodies; chimeric antibodies; Fab, Fab′, single chain V region fragments (scFv), fusion polypeptides, and unconventional antibodies.

The term “humanized antibody” refers to an antibody derived from a non-human (e.g., murine, rat, or rabbit) immunoglobulin, which has been engineered to contain minimal non-human (e.g., murine, rat, or rabbit) sequences. Typically, humanized antibodies are human immunoglobulins in which residues from the hypervariable complementarity determining region (CDR) are replaced by residues from the CDR of a non-human species (e.g., mouse, rat, rabbit, or hamster) that have a specificity, an affinity, and/or a capability of interest (Jones et al., 1986, Nature, 321:522-525; Riechmann et al., 1988, Nature, 332:323-327; Verhoeyen et al., 1988, Science, 239:1534-1536). Thus, the framework regions of humanized antibodies are essentially those of the human immunoglobulin. In some instances, the Fv framework region (FW) residues of a human immunoglobulin are replaced with the corresponding residues in an antibody from a non-human species that has a specificity, an affinity, and/or a capability of interest.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described, for example, in U.S. Pat. Nos. 5,225,539 or 5,639,641.

By “fragment” is meant a portion of a polypeptide or nucleic acid molecule. The “fragment” or “portion” contains, preferably, at least 10%, 20%, 30%, 40%, 50%, 60%, 70%, 80%, or 90% of the entire length of the reference nucleic acid molecule or polypeptide. In a particular embodiment, a fragment or portion of a polypeptide may contain 5, 10, 20, 30, 40, 50, 60, 70, 80, 90, 100, 200, or 300 amino acids. In embodiments, the fragment or portion retains the full or at least partial activity and/or function of the entire polypeptide or nucleic acid molecule.

“Detect” refers to identifying the presence, absence or amount of the analyte to be detected. In various embodiments, the analyte is a polypeptide or nucleic acid biomarker.

By “compete” in connection with an antibody, is meant, in general, that a first antibody, or an antigen-binding fragment thereof, vies for binding with a second antibody, or an antigen-binding fragment thereof, in which the binding of the first antibody (to its cognate antigen-binding site or epitope (e.g., on integrin αvβ8)) is detectably decreased in the presence of the second antibody compared to the binding of the first antibody in the absence of the second antibody. As may alternatively be the case, the binding of the second antibody to its cognate antigen-binding site or epitope is also detectably decreased in the presence of the first antibody; however, this is not always the case. Thus, a first antibody can inhibit the binding of a second antibody to its cognate antigen-binding site or epitope without the second antibody inhibiting the binding of the first antibody to its respective binding site or epitope on the antigen. However, where each antibody detectably inhibits the binding of the other antibody with its cognate epitope or ligand, whether to the same, or to a greater or lesser extent, the antibodies are said to “cross-compete” with each other for binding to their respective binding sites or epitope(s). Both competing and cross-competing antibodies are contemplated herein. Notwithstanding the mechanism by which antibody competition or cross-competition occurs, such as by steric hindrance, conformational change, or binding to a common binding site, epitope, or fragment thereof, and the like, both competing and/or cross-competing antibodies are encompassed herein and can be useful in disclosed methods.

The term “ameliorate” in connection with the treatments described herein refers to decreasing, reducing, diminishing, suppressing, attenuating, abrogating, arresting, inhibiting, blocking, neutralizing, or stabilizing the development or progression of a disease or condition, such as fibrosis in kidney cells and/or tissue (kidney fibrosis).

“Integrins” as referred to herein are cell-surface glycoproteins that are the principal receptors used by mammalian cells to bind to the extracellular matrix and mediate cell-cell and cell-extracellular matrix interactions. They are heterodimers (having α and β subunits bound noncovalently to each other) and function as transmembrane linkers between the extracellular matrix and the actin cytoskeleton of cells. Integrin proteins do not function as a passive glue, but rather are dynamic molecules that mediate the transfer of information across the cell membrane in both directions. Integrin-mediated adhesion can be regulated in response to signals by clustering and conformational changes triggered at integrins' cytoplasmic tails, which function as signal transducers to activate various intracellular signaling pathways when activated by ligand binding. In addition, integrin signaling controls cell survival, cell cycle progression, and differentiation. The regulation of integrin-mediated adhesion structures is critical for many forms of cell migration. Integrins also contribute to the pathogenesis of a diverse array of acquired and hereditary diseases.

There are several members of the integrin family of proteins, some of which have widespread tissue distribution. About twenty-four different integrins are present in vertebrates; a single cell may express multiple different types of integrin receptors on its surface. Human integrin β8 subunit, which is encoded by the ITGB8 gene, has ligands that include fibronectin and the TGF-β1 and TGF-β2 isoforms. In combination with the MT1 matrix metalloproteinase (MMP), αvβ8 integrin (a heterodimer comprising an alpha-V (αv) subunit associated with a beta-8 (β8) subunit as further described infra) is expressed on the cell surface and interacts with and mediates the activation of latent TGF-β in the cell matrix. The MT1 protease cleaves latent TGF-β to release the mature, active TGF-β polypeptide. Reactive oxygen species, other proteases, inflammation and pH change have also been demonstrated to be responsible for release of active TGFβ.

By “αvβ8” is meant an “αvβ8 integrin receptor,” “αvβ8 integrin,” or “integrin αvβ8” polypeptide or fragment thereof having at least about 85%, or greater, amino acid sequence identity to the human αvβ8 integrin amino acid sequence provided at NCBI Reference Sequence: NM_002214.2 and having αvβ8 activity and/or function as set forth below. Like other integrin beta (β) subunits, human αvβ8 contains an N-terminal signal peptide, a large extracellular domain that includes 4 cysteine-rich repeats, a transmembrane domain and a short C-terminal cytoplasmic domain. αvβ8 has a molecular mass of approximately 95 kD, consistent with substantial glycosylation of the predicted 81 kD (38 gene product. (M. Moyle et al., 1991, J. Biol. Chem., 266:19650-19658). Northern blot analysis has revealed that human αvβ8 is expressed as an approximately 8.5 kilobase (kb) mRNA in an osteosarcoma cell line. When expressed in mammalian cells, the β8 integrin subunit associates with the alpha-V (αV) subunit to form a cell surface αvβ8 integrin complex. In a particular embodiment, the polypeptide is human αvβ8 integrin. The term “αvβ8” as used herein is synonymous with “αvβ8 integrin receptor,” “αvβ8 integrin,” and “integrin αvβ8.” The designation “itgb8” typically refers to the human gene sequence of the β8 subunit.

The human β8 integrin (used interchangeably with the terms ITGB8, integrin beta-8, integrin β8, β8, and similar terms) protein sequence can be found at Uniprot accession number P26012 or NCBI Reference Sequence: NM_002214.2, as follows:

(SEQ ID NO: 16)
MCGSALAFFTAAFVCLQNDRRGPASFLWAAWVFSLVLGLGQGEDNRCASS
NAASCARCLALGPECGWCVQEDFISGGSRSERCDIVSNLISKGCSVDSIE
YPSVHVIIPTENEINTQVTPGEVSIQLRPGAEANFMLKVHPLKKYPVDLY
YLVDVSASMHNNIEKLNSVGNDLSRKMAFFSRDFRLGFGSYVDKTVSPYI
SIHPERIHNQCSDYNLDCMPPHGYIHVLSLTENITEFEKAVHRQKISGNI
DTPEGGFDAMLQAAVCESHIGWRKEAKRLLLVMTDQTSHLALDSKLAGIV
VPNDGNCHLKNNVYVKSTTMEHPSLGQLSEKLIDNNINVIFAVQGKQFHW
YKDLLPLLPGTIAGEIESKAANLNNLVVEAYQKLISEVKVQVENQVQGIY
FNITAICPDGSRKPGMEGCRNVTSNDEVLFNVTVTMKKCDVTGGKNYAII
KPIGFNETAKIHIHRNCSCQCEDNRGPKGKCVDETFLDSKCFQCDENKCH
FDEDQFSSESCKSHKDQPVCSGRGVCVCGKCSCHKIKLGKVYGKYCEKDD
FSCPYHHGNLCAGHGECEAGRCQCFSGWEGDRCQCPSAAAQHCVNSKGQV
CSGRGTCVCGRCECTDPRSIGRFCEHCPTCYTACKENWNCMQCLHPHNLS
QAILDQCKTSCALMEQQHYVDQTSECFSSPSYLRIFFIIFIVTFLIGLLK
VLIIRQVILQWNSNKIKSSSDYRVSASKKDKLILQSVCTRAVTYRREKPE
EIKMDISKLNAHETFRCNF.

The itgb8 polynucleotide coding sequence for human (38 integrin is presented below (8787 bp itgb8 mRNA nucleic acid sequence). The polynucleotide sequence of human (38 integrin can be found at accession number: NCBI Reference Sequence: NM_002214.2. A polynucleotide or fragment thereof having at least about 85% or greater nucleotide sequence identity to the itgb8 polynucleotide sequence encoding human (38 integrin polypeptide is encompassed by the disclosure.

| (SEQ ID NO: 17) | |
|---|---|
| 1 | ggcgggtgct tctagggcgc tcccagagcc gcctccccct gttgctggca tcccgagctt |
| 61 | cctcccttgc cagccaggac gctgccgact tgtctttgcc cgctgctccg cagacggggc |
| 121 | tgcaaagctg caactaatgg tgttggcctc cctgcccacc tgtggaagca actgcgctga |
| 181 | ttgatgcgcc acagactttt ttcccctcga cctcgccggc gtcccctccc acagatccag |
| 241 | catcacccag tgaatgtaca ttagggtggt ttccccccca gcttcgggct ttgtttgggt |
| 301 | ttgattgtgt ttggctcttc gctaagctga tttatgcagc agaagcccca ccggctggag |
| 361 | agaaacaaaa gctcttttct ttgtcccgga gcaggctgcg gagcccttgc agagccctct |
| 421 | ctccagtcgc cgccggggcc cttggccgtc gaaggaggtg cttctcgcgg agaccgcggg |
| 481 | acccgccgtg ccgagccggg agggccgcag gggccctgag atgccgagcg gtgcccgggc |
| 541 | ccgcttacct gcaccgcttg ctccgagccg cggggtccgc ctgctaggcc tgcggaaaac |
| 601 | gtcctagcga cactcggccc gcgggccccg aggtgcgccc gggaggcgcg agcccgcgtc |
| 661 | cggaaggcag tcaggcggcg ggcgcggggc gggctgtttt gcattatgtg cggctcggcc |
| 721 | ctggcttttt ttaccgctgc atttgtctgc ctgcaaaacg accggcgagg tcccgcctcg |
| 781 | ttcctctggg cagcctgggt gttttcactt gttcttggac tgggccaagg tgaagacaat |
| 841 | agatgtgcat cttcaaatgc agcatcctgt gccaggtgcc ttgcgctggg tccagaatgt |
| 901 | ggatggtgtg ttcaagagga tttcatttca ggtggatcaa gaagtgaacg ttgtgatatt |
| 961 | gtttccaatt taataagcaa aggctgctca gttgattcaa tagaataccc atctgtgcat |
| 1021 | gttataatac ccactgaaaa tgaaattaat acccaggtga caccaggaga agtgtctatc |
| 1081 | cagctgcgtc caggagccga agctaatttt atgctgaaag ttcatcctct gaagaaatat |
| 1141 | cctgtggatc tttattatct tgttgatgtc tcagcatcaa tgcacaataa tatagaaaaa |
| 1201 | ttaaattccg ttggaaacga tttatctaga aaaatggcat ttttctcccg tgactttcgt |
| 1261 | cttggatttg gctcatacgt tgataaaaca gtttcaccat acattagcat ccaccccgaa |
| 1321 | aggattcata atcaatgcag tgactacaat ttagactgca tgcctcccca tggatacatc |
| 1381 | catgtgctgt ctttgacaga gaacatcact gagtttgaga aagcagttca tagacagaag |
| 1441 | atctctggaa acatagatac accagaagga ggttttgacg ccatgcttca ggcagctgtc |
| 1501 | tgtgaaagtc atatcggatg gcgaaaagag gctaaaagat tgctgctggt gatgacagat |
| 1561 | cagacgtctc atctcgctct tgatagcaaa ttggcaggca tagtggtgcc caatgacgga |
| 1621 | aactgtcatc tgaaaaacaa cgtctatgtc aaatcgacaa ccatggaaca cccctcacta |
| 1681 | ggccaacttt cagagaaatt aatagacaac aacattaatg tcatctttgc agttcaagga |
| 1741 | aaacaatttc attggtataa ggatcttcta cccctcttgc caggcaccat tgctggtgaa |
| 1801 | atagaatcaa aggctgcaaa cctcaataat ttggtagtgg aagcctatca gaagctcatt |
| 1861 | tcagaagtga aagttcaggt ggaaaaccag gtacaaggca tctattttaa cattaccgcc |
| 1921 | atctgtccag atgggtccag aaagccaggc atggaaggat gcagaaacgt gacgagcaat |
| 1981 | gatgaagttc ttttcaatgt aacagttaca atgaaaaaat gtgatgtcac aggaggaaaa |
| 2041 | aactatgcaa taatcaaacc tattggtttt aatgaaaccg ctaaaattca tatacacaga |
| 2101 | aactgcagct gtcagtgtga ggacaacaga ggacctaaag gaaagtgtgt agatgaaact |
| 2161 | tttctagatt ccaagtgttt ccagtgtgat gagaataaat gtcattttga tgaagatcag |
| 2221 | ttttcttctg agagttgcaa gtcacacaag gatcagcctg tttgcagtgg tcgaggagtt |
| 2281 | tgtgtttgtg ggaaatgttc atgtcacaaa attaagcttg gaaaagtgta tggaaaatac |
| 2341 | tgtgaaaagg atgacttttc ttgtccatat caccatggaa atctgtgtgc tgggcatgga |
| 2401 | gagtgtgaag caggcagatg ccaatgcttc agtggctggg aaggtgatcg atgccagtgc |
| 2461 | ccttcagcag cagcccagca ctgtgtcaat tcaaagggcc aagtgtgcag tggaagaggc |
| 2521 | acgtgtgtgt gtggaaggtg tgagtgcacc gatcccagga gcatcggccg cttctgtgaa |
| 2581 | cactgcccca cctgttatac agcctgcaag gaaaactgga attgtatgca atgccttcac |
| 2641 | cctcacaatt tgtctcaggc tatacttgat cagtgcaaaa cctcatgtgc tctcatggaa |
| 2701 | caacagcatt atgtcgacca aacttcagaa tgtttctcca gcccaagcta cttgagaata |
| 2761 | tttttcatca ttttcatagt tacattcttg attgggttgc ttaaagtcct gatcattaga |
| 2821 | caggtgatac tacaatggaa tagtaataaa attaagtcct catcagatta cagagtgtca |
| 2881 | gcctcaaaaa aggataagtt gattctgcaa agtgtttgca caagagcagt cacctaccga |
| 2941 | cgtgagaagc ctgaagaaat aaaaatggat atcagcaaat taaatgctca tgaaactttc |
| 3001 | aggtgcaact tctaaaaaaa gatttttaaa cacttaatgg gaaactggaa ttgttaataa |
| 3061 | ttgctcctaa agattataat tttaaaagtc acaggaggag acaaattgct cacggtcatg |
| 3121 | ccagttgctg gttgtacact cgaacgaaga ctgacaagta tcctcatcat gatgtgactc |
| 3181 | acatagctgc tgactttttc agagaaaaat gtgtcttact actgtttgag actagtgtcg |
| 3241 | ttgtagcact ttactgtaat atataactta tttagatcag catagaatgt agatcctctg |
| 3301 | aagagcactg attacacttt acaggtacct gttatcccta cgcttcccag agagaacaat |
| 3361 | gctgtgagag agtttagcat tgtgtcacta caagggtaca gtaatccctg cactggacat |
| 3421 | gtgaggaaaa aaataatctg gcaagtatat tctaaggttg ccaaacactt caacagttgg |
| 3481 | tggttgaata gacaagaaca gctagatgaa taaatgattc gtgtttcact ctttcaagag |
| 3541 | gtgaacagat acaaccttaa tcttaaaaga ttattgcttt ttaaagtgtg tagttttatg |
| 3601 | catgtgtgtt tatggtttgc ttatttttgc aagatggata ctaattccag cattctctcc |
| 3661 | tctttgcctt tatgttttgt tttctttttt acaggataag tttatgtatg tcacagatga |
| 3721 | ctggattaat taagtgctaa gttactactg ccataaaaaa ctaataatac aatgtcactt |
| 3781 | tatcagaata ctagttttaa aagctgaatg ttaatagggg acactgtaaa gtatcatcaa |
| 3841 | aacctgaata gcttcattgt gcacaagtgt ggagttttgt atcctcttac ctggtaaact |
| 3901 | gaagggattg tttggccatt tcatttatct tatcattaat tcacaagata gttagaaatt |
| 3961 | ctgcctcaag caaagtacca cattttgaat gttttcttag attttgattg caagtagata |
| 4021 | tcagcatttt ttaaatgaaa agctatatta tcttctccct tcaaggcagc ctaaggatgt |
| 4081 | tctttcccag aatcactcca acccttcttg ccagaattca taaaagtaca aaattggaga |
| 4141 | atagatgata tcttagaaat aagctttttt tttttttttt tttttttttg agacggagtt |
| 4201 | tcactcttgt cacccaggct gaagtgcaat ggcgcaatta gggttcactg caacctctgc |
| 4261 | ctcccgggtt caagcagttc tcctgcctca gcctcctgag tagctgggat tacaggcatc |
| 4321 | caccaccgtg cccagctaat ttttgtattt ttagtagaga cggggttttg ccatgttgga |
| 4381 | caggttgatc tcaaactcct gacctcaggt gatctaccct cctcggcctc ccagagtgtt |
| 4441 | gggattacag gcatgagcca ccatgccagg ctgctaattc tcctttttag tgagttaggg |
| 4501 | aactgagcct cagaaaactt aaacgatttc tcagaaaaca ctcaagtgat aaagtggcca |
| 4561 | cattggaaag gagtttttat cttctcattg tcaggccagt gttcattgca caatatcatg |
| 4621 | ctacctcttg aatctttaaa atattcaatt ggcaaatgtt tttcaatgtg atttactcat |
| 4681 | gtcttaagtg tatgaggaaa gttcaaagca aaatagaaag gaataattca aactgaattg |
| 4741 | tccataatca gcttccagtc tttcatgcta atcagcttct taagagactg aagtatggca |
| 4801 | tacctacagg ggaattcctt cgcaccatag cctgtatgaa cagtgttccc tggagttctc |
| 4861 | cagtgctcag cttgagacct tgatacacgg gccatgagcc ctgtcttccc caatggaaat |
| 4921 | ttatttacac ttaccttatc cctatggact tagtctgatt ttattggcta ggagtctaac |
| 4981 | agtcctgtgt ggatatacag ttttgcccat gacaacaaag gaatctatcc gaaatatctt |
| 5041 | tttttttata ataaacttcc aagatttgct gtcttccagc acttgagtta aagtactaga |
| 5101 | tactgcattt tgatgaagac taaccccatc tcatattcta ccctaaagag aactgaaaaa |
| 5161 | cctataataa gttgttctgg agccaataaa cacagcagct ctgttagatg tcctctacag |
| 5221 | ccaagcactt tcaatgctaa cttgaactgc atttccttcc tcaaatgaga gattgacata |
| 5281 | attcagtact gtgagtcact tgtataagaa acctttgatc actaaaaata atgtaaaaat |
| 5341 | tgggtttagt agcctaatac acataacgtt cttcttaaaa aggaaaatgg atggatgcct |
| 5401 | gacaaccctc caaaagaaaa aagtgtaaga tagccattaa gatgatgaca atttttgaaa |
| 5461 | tgaacattat gatatttatg aacaataaac aaatttccgt atggaatgaa ttatccaaaa |
| 5521 | agagtataac aaaatgaaat ccttaaaaat ccagagttta tatttttttt ataccctcac |
| 5581 | ttgtttgcac taactttata gtggaccaag gctgttacca taggaaggga caaacttcct |
| 5641 | tgtaggcaac tcagtgttag acgatgattg tggttatgct tgcaaagtct tgtgcttatc |
| 5701 | ttttttgttt ttacttaaaa agctaatttt taaagattgt agggcttgta ttttacttga |
| 5761 | ataattgata tcttcctgtg taatgatttg tgagatgaga attaatattt gactagttag |
| 5821 | aattaattaa atggtaaggg aacacagggt actcttaggt taaataatgt atgcaaatag |
| 5881 | agtctatttt caactaatat ggccacagga gccttttgag attcattgat attaaacaca |
| 5941 | attaatgaaa ttttaaattg ttaacagaat tgagaacttg aacaacactt ttagtactgc |
| 6001 | agcatttttg tgccctaaag tatgtaatga tttataaatg tgccatacat acactacaac |
| 6061 | ataacatttg ctttgttatg cattttattt ctctggggac accattgcac tgcagtgcac |
| 6121 | acgtatttat aaacatttgt tatatttttg gaaacttgct aatatttatt aagtcataga |
| 6181 | cttttctgga ggacttaaaa attcactaaa aatctgatta tgtcttaaat gttcagttta |
| 6241 | tctttggttt attaaaataa aaaaaaaatc taagattaaa cacagtagat atctctggag |
| 6301 | gcaattttcc aaaactcaac attaaaattt gtggatgcat gagatgcaat ccttcaaaga |
| 6361 | atgaatctga aatatatttt taatatttac ttaatatcca ctgaagatat ctttatgcaa |
| 6421 | gacaagagtc agccatcaga cactgaaata tattatgata gattatgaag aattttctct |
| 6481 | gtagaattat attcttcctg gaacctggta gagtagatta gactcaaagg ctttttcttc |
| 6541 | cttttcttac tcctgttttt tccactcact cttcccaaga gatttcctaa agcttcaagc |
| 6601 | ttaataagcc taatagtgaa aaataactga atttaatggt ataatgaagt tcttcatttc |
| 6661 | cagacatctt taattgatct taaagctcat ttgagtcttt gcccctgaac aaagacagac |
| 6721 | ccattaaaat ctaagaattc taaattttca caactgtttg agcttctttt cattttgaag |
| 6781 | gatttggaat atatatgttt tcataaaagt atcaagtgaa atatagttac atgggagctc |
| 6841 | aatcatgtgc agattgcatt ctgttatgtt gactcaatat ttaatttaca actatcctta |
| 6901 | tttatattga cctcaagaac tccattttat gcaatgcaga ccactgagat atagctaaca |
| 6961 | ttctttcaaa taattttcct tttcttttat aattcctcta tagcaaattt ttatgtataa |
| 7021 | ctgattatac atatccatat ttatatttca ttgattccaa gacatcactt tttcaattta |
| 7081 | acatctctga aattgtgaca tttcttgcaa ctgttggcac ttcagatgca gtgtttaaaa |
| 7141 | ttatgcttga ataaatatta cactaatcca actttaccta aatgtttatg catctaggca |
| 7201 | aattttgttt tcttataaag atttgagagc ccatttatga caaaatatga aggcgaaatt |
| 7261 | taaggacaac tgagtcacgc acaactcaac atggagccta actgattatc agctcagatc |
| 7321 | ccgcatatct tgagtttaca aaagctcttt caggtcccca tttatacttt acgtgagtgc |
| 7381 | gaatgatttc agcaaaccct aacttaacta acaagaatgg gtaggtatgt ctacgtttca |
| 7441 | ttaacaaatt tttattattt ttattctatt atatgagatc cttttatatt atcatctcac |
| 7501 | ttttaaacaa aattaactgg aaaaatatta catggaactg tcatagttag gttttgcagc |
| 7561 | atcttacatg tcttgtatca atggcaggag aaaaatatga taaaaacaat cagtgctgtg |
| 7621 | aaaaacaact ttcttctaga gtcctcttac tttttattct tctttatcat ttgtgggttt |
| 7681 | ttcccccttg gctctgatca ctttaacttc aagcttatgt aacgactgtt ataaaactgc |
| 7741 | atatttaaat tatttgaatt atatgaaata attgttcagc tatctgggca gctgttaatg |
| 7801 | taaacctgag agtaataaca ctactctttt atctacctgg aatacttttc tgcataaaat |
| 7861 | ttatctttgt aagctaactc tattaatcag gtttcttcta gcctctgcaa cctacttcag |
| 7921 | ttagaattgt ctaatactgc tctattaatc aggtttctag cctctacaac ctacttcagt |
| 7981 | taaaattgtc taatacagca atatttaaaa aaaaaacact gcaattgtca aggatggaaa |
| 8041 | atgtgtgatt tgtgtaaaca atttttacca actttacatt ttcctacaga taaatgtgaa |
| 8101 | attttgataa gaagtctacg caatgacaag tatggtacat aaattttatt aagaatattg |
| 8161 | agtataaagt actttaattc taaattataa gaaaatatac atttgcacat attaatatag |
| 8221 | aaattcattt tgtgtatatt taacatagct tttaaactat tttacattag ctacttcatt |
| 8281 | atggtttctt gaacttctga aaaaaattag aaatgtatta aacttatcag taacataaaa |
| 8341 | acttattttg tttcacctaa cgaatactgc gtttgtaaaa ataaatttaa tatagaatat |
| 8401 | atttttaaat taaatatttg aatataaaat agctctaaga aagaagcaaa ttatcactga |
| 8461 | acatatttct tattatttct ggctttgaat tatacgtaac ttaaattgtc ttaaatgata |
| 8521 | cagaatattg gagaatatga tactttcaca taatatacta tgaacctgtt catataactc |
| 8581 | tgattgacta ctaacttctg ttttatgtat ttattaaaga gctgacactg tagtttgtgg |
| 8641 | tgagatgttt atttttctaa cagagcttat aacagttagg acaaggcatt taattaatgc |
| 8701 | atcattctgt ttagtagtag gtgttaatca atatgaaatt ctctgtttta aaataaaaat |
| 8761 | gtaaaaatct aagaataaaa aaaaaaa |

The alpha-V integrin (α-V, ITGAV) subunit (also called alpha-V and αv) associates with either the β-1 (ITGB1), β-3 (ITGB3), β-5 (ITGB5), β-6 (ITGB6) or β-8 (ITGB8) subunits, forming a heterodimer of an alpha (αv) and a beta (β1-8) subunit. The alpha subunit is composed of a heavy (Integrin α-V heavy chain) and a light chain (Integrin α-V light chain) linked by a disulfide bond. In a particular embodiment, the αv integrin subunit associates with the β8 integrin subunit, forming the αvβ8 integrin. The human α-V (ITAV), which can associate with β-8 (ITGB8) as set forth supra, comprises 1040 amino acids and can be found at Uniprot (UniProtKB) Accession No. P06756, as follows:

```
(SEQ ID NO: 18)
                10 20 30 40
MAFPPRRRLR LGPRGLPLLL SGLLLPLCRA FNLDVDSPAE
                50 60 70 80
YSGPEGSYFG FAVDFFVPSA SSRMFLLVGA PKANTTQPGI
                90 100 110 120
VEGGQVLKCD WSSTRRCQPI EFDATGNRDY AKDDPLEFKS
              130 140 150 160
HQWFGASVRS KQDKILACAP LYHWRTEMKQ EREPVGTCFL
              170 180 190 200
QDGTKTVEYA PCRSQDIDAD GQGFCQGGFS IDFTKADRVL
              210 220 230 240
LGGPGSFYWQ GQLISDQVAE IVSKYDPNVY SIKYNNQLAT
              250 260 270 280
RTAQAIFDDS YLGYSVAVGD FNGDGIDDFV SGVPRAARTL
              290 300 310 320
GMVYIYDGKN MSSLYNFTGE QMAAYFGFSV AATDINGDDY
              330 340 350 360
ADVFIGAPLF MDRGSDGKLQ EVGQVSVSLQ RASGDFQTTK
              370 380 390 400
LNGFEVFARF GSAIAPLGDL DQDGFNDIAI AAPYGGEDKK
              410 420 430 440
GIVYIFNGRS TGLNAVPSQI LEGQWAARSM PPSFGYSMKG
              450 460 470 480
ATDIDKNGYP DLIVGAFGVD RAILYRARPV ITVNAGLEVY
              490 500 510 520
PSILNQDNKT CSLPGTALKV SCFNVRFCLK ADGKGVLPRK
              530 540 550 560
LNFQVELLLD KLKQKGAIRR ALFLYSRSPS HSKNMTISRG
              570 580 590 600
GLMQCEELIA YLRDESEFRD KLTPITIFME YRLDYRTAAD
              610 620 630 640
TTGLQPILNQ FTPANISRQA HILLDCGEDN VCKPKLEVSV
              650 660 670 680
DSDQKKIYIG DDNPLTLIVK AQNQGEGAYE AELIVSIPLQ
              690 700 710 720
ADFIGVVRNN EALARLSCAF KTENQTRQVV CDLGNPMKAG
              730 740 750 760
TQLLAGLRFS VHQQSEMDTS VKFDLQIQSS NLFDKVSPVV
              770 780 790 800
SHKVDLAVLA AVEIRGVSSP DHVFLPIPNW EHKENPETEE
              810 820 830 840
DVGPVVQHIY ELRNNGPSSF SKAMLHLQWP YKYNNNTLLY
              850 860 870 880
ILHYDIDGPM NCTSDMEINP LRIKISSLQT TEKNDTVAGQ
              890 900 910 920
GERDHLITKR DLALSEGDIH TLGCGVAQCL KIVCQVGRLD
              930 940 950 960
RGKSAILYVK SLLWTETFMN KENQNHSYSL KSSASFNVIE
              970 980 990 1000
FPYKNLPIED ITNSTLVTTN VTWGIQPAPM PVPVWVIILA
            1010 1020 1030 1040
VLAGLLLLAV LVFVMYRMGF FKRVRPPQEE QEREQLQPHE
NGEGNSET
```

The polynucleotide coding sequence encoding the human alpha-V (ITGAV) is presented below (3147 nucleotide bp). The polynucleotide sequence of human αv integrin (CCDS 2292.1) can be found at accession number: NCBI Reference Sequence: NM_002210.4. A polynucleotide or fragment thereof having at least about 85% or greater nucleotide sequence identity to the αv (ITGAV) integrin polynucleotide sequence encoding human ITGAV integrin is encompassed by the disclosure.

(SEQ ID NO: 19)
atggcttttccgccgcggcgacggctgcgcctcggtccccgcggcctccc
gcttcttctctcgggactcctgctacctctgtgccgcgccttcaacctag
acgtggacagtcctgccgagtactctggccccgagggaagttacttcggc
ttcgccgtggatttcttcgtgcccagcgcgtcttcccggatgtttcttct
cgtgggagctcccaaagcaaacaccacccagcctgggattgtggaaggag
ggcaggtcctcaaatgtgactggtcttctacccgccggtgccagccaatt
gaatttgatgcaacaggcaatagagattatgccaaggatgatccattgga
atttaagtcccatcagtggtttggagcatctgtgaggtcgaaacaggata
aaattttggcctgtgccccattgtaccattggagaactgagatgaaacag
gagcgagagcctgttggaacatgctttcttcaagatggaacaaagactgt
tgagtatgctccatgtagatcacaagatattgatgctgatggacagggat
tttgtcaaggaggattcagcattgattttactaaagctgacagagtactt
cttggtggtcctggtagcttttattggcaaggtcagcttatttcggatca
agtggcagaaatcgtatctaaatacgaccccaatgtttacagcatcaagt
ataataaccaattagcaactcggactgcacaagctatttttgatgacagc
tatttgggttattctgtggctgtcggagatttcaatggtgatggcataga
tgactttgtttcaggagttccaagagcagcaaggactttgggaatggttt
atatttatgatgggaagaacatgtcctccttatacaattttactggcgag
cagatggctgcatatttcggattttctgtagctgccactgacattaatgg
agatgattatgcagatgtgtttattggagcacctctcttcatggatcgtg
gctctgatggcaaactccaagaggtggggcaggtctcagtgtctctacag
agagcttcaggagacttccagacgacaaagctgaatggatttgaggtctt
tgcacggtttggcagtgccatagctcctttgggagatctggaccaggatg
gtttcaatgatattgcaattgctgctccatatgggggtgaagataaaaaa
ggaattgtttatatcttcaatggaagatcaacaggcttgaacgcagtccc
atctcaaatccttgaagggcagtgggctgctcgaagcatgccaccaagct
ttggctattcaatgaaaggagccacagatatagacaaaaatggatatcca
gacttaattgtaggagcttttggtgtagatcgagctatcttatacagggc
cagaccagttatcactgtaaatgctggtcttgaagtgtaccctagcattt
taaatcaagacaataaaacctgctcactgcctggaacagctctcaaagtt
tcctgttttaatgttaggttctgcttaaaggcagatggcaaaggagtact
tcccaggaaacttaatttccaggtggaacttcttttggataaactcaagc
aaaagggagcaattcgacgagcactgtttctctacagcaggtccccaagt
cactccaagaacatgactatttcaagggggggactgatgcagtgtgagga
attgatagcgtatctgcgggatgaatctgaatttagagacaaactcactc
caattactatttttatggaatatcggttggattatagaacagctgctgat
acaacaggcttgcaacccattcttaaccagttcacgcctgctaacattag
tcgacaggctcacattctacttgactgtggtgaagacaatgtctgtaaac
ccaagctggaagtttctgtagatagtgatcaaaagaagatctatattggg
gatgacaaccctctgacattgattgttaaggctcagaatcaaggagaagg
tgcctacgaagctgagctcatcgtttccattccactgcaggctgatttca
tcggggttgtccgaaacaatgaagccttagcaagactttcctgtgcattt
aagacagaaaaccaaactcgccaggtggtatgtgaccttggaaacccaat
gaaggctggaactcaactcttagctggtcttcgtttcagtgtgcaccagc
agtcagagatggatacttctgtgaaatttgacttacaaatccaaagctca
aatctatttgacaaagtaagcccagttgtatctcacaaagttgatcttgc
tgttttagctgcagttgagataagaggagtctcgagtcctgatcatgtct
ttcttccgattccaaactgggagcacaaggagaaccctgagactgaagaa
gatgttgggccagttgttcagcacatctatgagctgagaaacaatggtcc
aagttcattcagcaaggcaatgctccatcttcagtggccttacaaatata
ataataacactctgttgtatatccttcattatgatattgatggaccaatg
aactgcacttcagatatggagatcaaccctttgagaattaagatctcatc
tttgcaaacaactgaaaagaatgacacggttgccgggcaaggtgagcggg
accatctcatcactaagcgggatcttgccctcagtgaaggagatattcac
actttgggttgtggagttgctcagtgcttgaagattgtctgccaagttgg
gagattagacagaggaaagagtgcaatcttgtacgtaaagtcattactgt
ggactgagacttttatgaataaagaaaatcagaatcattcctattctctg
aagtcgtctgcttcatttaatgtcatagagtttccttataagaatcttcc
aattgaggatatcaccaactccacattggttaccactaatgtcacctggg
gcattcagccagcgcccatgcctgtgcctgtgtgggtgatcattttagca
gttctagcaggattgttgctactggctgttttggtatttgtaatgtacag
gatgggcttttttaaacgggtccggccacctcaagaagaacaagaaaggg
agcagcttcaacctcatgaaaatggtgaaggaaactcagaaacttaa.

A humanized anti-αvβ8 integrin antibody, referred to as “MEDI-hu37E1B5” herein is useful in the disclosed compositions and methods. The MEDI-hu37E1B5 antibody has the heavy and light chain variable region amino acid sequences presented below. This antibody is specific and selective for binding to αvβ8 integrin protein that is expressed on kidney cells and tissue, and, more particularly, that is highly expressed on diseased kidney cells and tissue, such as fibrotic kidney tissue, in a subject having kidney disease such as chronic kidney disease (CKD). In an embodiment, an anti-αvβ8 integrin antibody, or an antigen-binding fragment thereof, having at least about or at least 85%, or greater, amino acid sequence identity to the amino acid sequences of the heavy chain variable region (V_{H}), (116 amino acid residues), and light chain variable region (V_{L}), (107 amino acid residues), of the MEDI-hu37E1B5 αvβ8 integrin antibody, set forth below, is encompassed by the disclosure.

### V_{H} Amino Acid Sequence of the MEDI-hu37E1B5 Anti-αvβ8 Integrin Antibody:

(SEQ ID NO: 7)
EVQLVESGGGLVQPGGSLRLSCAVSGFVFSRYWMSWVRQAPGKGLEWIGE
INPDSSTINYTSSLKDRFTISRDNAKNSLYLQMNSLRAEDTAVYYCAILI
TTEDYWGQGTTVTVSS

### V_{L} Amino Acid Sequence of the MEDI-hu37E1B5 Anti-αvβ8 Integrin Antibody:

(SEQ ID NO: 8)
DIQLTQSPSSLSASVGDRVTITCKASQDINSYLSWFQQKPGKAPKSLIYY
ANRLVDGVPSRFSGSGSGTDFTLTISSLQPEDFATYYCLQYDEFPYTFGG
GTKVEIK

In the above V_{H} and V_{L} sequences of the MEDI-hu37E1B5 antibody, the three CDR regions (as defined by Kabat) are underlined. More specifically, the amino acid sequences of the three CDRs of the heavy chain variable region (V_{H}) of the MEDI-hu37E1B5 antibody are as follows:

V_{H} CDR1:
(SEQ ID NO: 1)
RYWMS
V_{H} CDR2:
(SEQ ID NO: 2)
EINPDSSTINYTSSL
V_{H} CDR3:
(SEQ ID NO: 3)
LITTEDY

The amino acid sequences of the three CDRs of the light chain variable region (V_{L}) of the MEDI-hu37E1B5 antibody are as follows:

V_{L} CDR1:
(SEQ ID NO: 4)
KASQDINSYLS
V_{L} CDR2:
(SEQ ID NO: 5)
YANRLVD
V_{L} CDR3:
(SEQ ID NO: 6)
LQYDEFPYT

Also encompassed by the disclosure is a polynucleotide sequence encoding the V_{H} and V_{L} regions of the MEDI-hu37E1B5 anti-αvβ8 integrin antibody identified above, or an antigen binding fragment thereof. In an embodiment, a polynucleotide sequence encoding the V_{H} and V_{L} regions of the MEDI-hu37E1B5 anti-αvβ8 integrin antibody noted above, or an antigen binding fragment thereof having at least about or at least 85%, or greater, nucleotide sequence identity to the MEDI-hu37E1B5 nucleotide sequence is also encompassed by the disclosure.

Another anti-αvβ8 integrin antibody, called “B5-15” herein, is particularly useful in the compositions and methods disclosed herein. The B5-15 anti-αvβ8 integrin antibody is a humanized and affinity optimized antibody (of the IgG1 isotype) that specifically binds to αvβ8 integrin and has the heavy and light chain variable region amino acid sequences presented below. The humanized B5-15 antibody was derived and affinity optimized from the above-described MEDI-hu37E1B5 antibody, which is the “parent” of the B5-15 antibody, as described herein. The B5-15 antibody is highly specific and selective for binding to αvβ8 integrin, particularly, αvβ8 integrin expressed on kidney cells and tissue, and, more particularly, to αvβ8 integrin that is highly expressed on diseased kidney cells and tissue, such as fibrotic kidney tissue in a subject having kidney disease such as chronic kidney disease (CKD). In an embodiment, an anti-αvβ8 integrin antibody, or an antigen-binding fragment thereof, having at least about or at least 85%, or greater, amino acid sequence identity to the amino acid sequences of the heavy chain variable region (V_{H}), (116 amino acid residues), and light chain variable region (V_{L}), (107 amino acid residues), of the B5-15 αvβ8 integrin antibody, set forth below, is encompassed by the disclosure.

### V_{H} Amino Acid Sequence of the Humanized and Optimized B5-15 Anti-αvβ8 Integrin Antibody:

(SEQ ID NO: 12)
EVQLVESGGGLVQPGGSLRLSCAVSGFVFSRSWISWVRQAPGKGLEWIGEI
NPDSSTINYTSSLKDRFTISRDNAKNSLYLQMNSLRAEDTAVYYCAILITT
EDYWGQGTTVTVSS

### V_{L} Amino Acid Sequence of the Humanized and Optimized B5-15 Anti-αvβ8 Integrin Antibody:

(SEQ ID NO: 13)
DIQLTQSPSSLSASVGDRVTITCKASQDINKYLSWFQQKPGKAPKSLIYYA
NRLVDGVPSRFSGSGSGTDFTLTISSLQPEDFATYYCLQYDVFPYTFGGGT
KVEIK

In the above V_{H} and V_{L} sequences of the B5-15 humanized and affinity optimized antibody, the amino acid sequences of the three CDR regions (as defined by Kabat) are underlined. More specifically, the amino acid sequences of the three CDRs of the heavy chain variable region (V_{H}) of the B5-15 antibody are as follows:

V_{H} CDR1:
(SEQ ID NO: 9)
RSWIS
V_{H} CDR2:
(SEQ ID NO: 2)
EINPDSSTINYTSSL
V_{H} CDR3:
(SEQ ID NO: 3)
LITTEDY

The amino acid sequences of the three CDRs of the light chain variable region (V_{L}) of the B5-15 humanized and affinity optimized antibody are as follows:

V_{L} CDR1:
(SEQ ID NO: 10)
KASQDINKYLS
V_{L} CDR2:
(SEQ ID NO: 5)
YANRLVD
V_{L} CDR3:
(SEQ ID NO: 11)
LQYDVFPYT

Also encompassed by the disclosure is a polynucleotide sequence encoding the V_{H} and V_{L} regions of the B5-15 anti-αvβ8 integrin antibody noted above, or an antigen binding fragment thereof. Provided below is a polynucleotide sequence encoding the V_{H} region of the optimized B5-15 anti-αvβ8 integrin antibody:

(SEQ ID NO: 14)
gaggtgcagctggtggaaagcggcggaggactggtgcagcctggcggcagc
ctgagactgagctgcgccgtgtccggcttcgtgttcagccggagctggatc
agctgggtccgccaggccccagggaagggcctggaatggatcggcgagatc
aaccccgacagcagcaccatcaactacaccagcagcctgaaggaccggttc
accatcagccgggacaacgccaagaacagcctgtacctgcagatgaacagc
ctgcgggccgaggacaccgccgtgtactactgcgccatcctcatcaccacc
gaggactactggggccagggcaccaccgtgaccgtgtcctct.

Provided below is a polynucleotide sequence encoding the V_{L} (kappa) region of the optimized B5-15 anti-αvβ8 integrin antibody:

(SEQ ID NO: 15)
gacatccagctgacccagagccccagcagcctgagcgccagcgtgggcgac
agagtgaccatcacatgcaaggccagccaggacatcaacaagtacctgagc
tggttccagcagaagcccggcaaggcccccaagagcctgatctactacgcc
aaccggctggtggacggcgtgcccagcagattttctggcagcggcagcggc
accgacttcaccctgaccatcagcagcctgcagcccgaggacttcgccacc
tactactgcctgcagtacgacgtgttcccctacaccttcggcggaggcacc
aaggtggaaatcaag.

In an embodiment, a polynucleotide sequence encoding the V_{H} and V_{L} regions of the B5-15 anti-αvβ8 integrin antibody noted above, or an antigen binding fragment thereof, having at least about or at least 85%, or greater, nucleotide sequence identity to the B5-15 nucleotide sequence is also encompassed by the disclosure.

The cytokine, transforming growth factor-beta (β), (TGF-β), is a multifunctional regulator that modulates cell proliferation, differentiation, apoptosis, adhesion and migration of various cell types. TGF-β induces the production of extracellular matrix (ECM) proteins and almost all cell types, e.g., activated T and B cells, hematopoietic cells, macrophages, dendritic cells, produce TGF-β and/or are sensitive to its effects. (S. Dennler et al., 2002, J. Leukoc. Biol., 71:731-740). TGF-β is a member of a diverse superfamily that includes greater than 30 related members in mammals, viz, 3 TGF-β isoforms, 4 activins, and over 20 Bone Morphogenic proteins (BMPs). The 3 mammalian isoforms of TGF-β (TGF-β1, TGF-β2 and TGF-(33) share 70-82% homology at the amino acid level and have qualitatively similar activities in different systems. The active form of TGF-β is a dimer stabilized by hydrophobic interactions, which are further strengthened by an intersubunit disulfide bridge, in most cases. The TGF-β1 isoform is the most abundant isoform in renal cells.

The mechanism by which TGF-β initiates intracellular signaling at the cell membrane is generally well understood. See, e.g., I. Loeffler and G. Wolf, 2013, Nephrol. Dial. Transplant, 29:i37-i45). The intracellular mediators of TGF-β signaling are called Smads, which act downstream of the type 1 TGF-β receptor, TβR-1, and which are categorized into three classes. The receptor-regulated Smads (R-Smads), e.g., Smad1, Smad2, Smad3, Smad5 and Smad8, which are directly phosphorylated and activated by TβR-1 (which is a transmembrane receptor serine/threonine kinase), form hetero-oligomeric complexes with a second class of Smad, the common mediator Smads (Co-Smads), e.g., Smad4. These Smad complexes translocate into the nucleus where they interact with site-specific DNA transcription factors and participate in the regulation of target genes. Smad2 and Smad3 respond to signaling by the TGF-β subfamily. A third identified class of Smads includes the inhibitory Smads Smad6 and Smad7, which antagonize the activity of the receptor-regulated Smads by physically interacting with the activated TβR-1 receptor and can prevent the docking and phosphorylation of the R-Smads. (Ibid.). By virtue of its pleiotropic effects, TGF-β can also directly activate other signal transduction cascades, including MAPK pathways, such as Ras, Raf, Erk, JNK and p38, in addition to Smad-mediated transcription. Moreover, TGF-β can activate the phosphatidylinositol-3-kinase (PI-3K) cascade by phosphorylation of its effector Akt, as well as Rho-like GTPases, including RhoA, Rac and cdc42. (Ibid.).

TGF-β is synthesized by a number of renal cell types and exerts its biological (and pathophysiological) effects through the above-noted signaling pathways. TGF-β is upregulated in renal diseases and induces renal cells to produce extracellular matrix proteins, which leads to glomerulosclerosis and tubule-interstitial (TI) fibrosis, which is characterized as a progressive, detrimental connective tissue deposition on the kidney parenchyma and is a damaging process, leading to the deterioration of renal function. Different types of renal cells undergo different pathophysiological changes induced by the activity of TGF-β, leading to apoptosis, tissue hypertrophy and podocyte foot processes abnormalities, ultimately causing renal dysfunction. (Ibid.).

As used herein, the terms “determining”, “evaluating,” “assessing”, “assaying”, “measuring” and “detecting”, and “identifying” refer to both quantitative and qualitative determinations, and as such, the term “determining” is used interchangeably herein with “assaying,” “measuring,” and the like. Where a quantitative determination is intended, the phrase “determining an amount, level, or concentration” of an analyte, substance, protein, and the like is used. Where a qualitative and/or quantitative determination is intended, the phrase “determining a level” of an analyte or “detecting” an analyte is used.

By “disease” is meant any condition or disorder that damages, interferes with or dysregulates the normal function of a cell, tissue, or organ. Diseases of and associated with the kidney as referred to herein include, by way of nonlimiting example, diabetic nephropathy (DN), chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like. Such diseases, conditions, pathologies and/or the symptoms thereof associated with the kidney may be acute or chronic in a subject and are not intended to be limiting.

In general terms, “fibrosis” is the formation of excess connective tissue in an organ or tissue that can occur as a result of a reactive (e.g., response to injury; disease) or reparative process. Fibrosis can occur in a reactive, benign, or a pathological state. In response to injury, fibrosis can be called scarring. Renal scarring results in a progressive loss of renal function, ultimately leading to end-stage renal failure and a requirement for dialysis or kidney transplantation.

Renal fibrosis is the inevitable consequence of an excessive accumulation of extracellular matrix that occurs in virtually every type of chronic kidney disease. The pathogenesis of renal fibrosis is a progressive process that ultimately leads to end-stage renal disease/failure, a devastating disorder that requires dialysis or kidney transplantation. In general, renal fibrosis represents a failed wound-healing process of the kidney tissue after chronic, sustained injury or damage. Several cellular pathways, including mesangial and fibroblast cell activation, as well as tubular epithelial-mesenchymal transition (EMT), have been identified as the primary ways in which matrix-producing cells are produced in diseased conditions. (See, e.g., Y. Liu, 2006, Kidney Int., 69(2):213-217).

Among the many fibrogenic factors that regulate renal fibrotic process, TGF-β plays a central role. Although defective matrix degradation may contribute to tissue scarring, the exact action and mechanisms of the matrix-degrading enzymes in the injured kidney are complex and not well understood. Intervening with the activities of endogenous anti-fibrotic factors may provide strategies for antagonizing the fibrogenic action of the TGF-β/Smad signaling pathways.

“Podocytes” are highly specialized epithelial cells in the Bowman's capsule in the kidneys that wrap around capillaries of the glomerulus. It is the foot processes or projections of podocytes that wrap around the capillaries and produce filtration slits (or slit diaphragms) through which blood and blood components are filtered. The Bowman's capsule filters blood and retains larger molecules (e.g., proteins) while filtering smaller molecules (e.g., water, salts, sugars) as the first step in the formation of urine. Together with endothelial cells of the glomerular capillary loop and the glomerular basement membrane, podocytes form a filtration barrier. Podocytes and mesangial cells of the kidney support the structure and function of the glomerulus.

The “glomerulus” in the kidney is a network or cluster of capillaries, called a tuft, situated inside a cup-like sac (glomerular capsule) located at the end of each kidney tubule (nephron) and is involved in the filtration of blood. The composition of the glomerular capillary wall determines what and how much is filtered into the glomerular capsule. The capillary walls are composed of an endothelium layer having relatively large pores through which solutes, plasma proteins and fluids can pass, but not blood cells; a basement membrane layer, which is fused to the endothelial layer and which prevents plasma proteins from being filtered out of the blood; and an epithelial layer, which consists of podocytes that are attached to the basement membrane by their foot processes. Fluid passes through the filtration slits formed by the podocytes. A thin diaphragm between the slits serves as a final filtration barrier before fluid enters the glomerular space.

The terms “isolated,” “purified” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified, as used herein, if it is substantially free of cellular material, viral contaminants, or culture medium when produced by recombinant DNA techniques, or chemical precursors, or other chemicals when chemically synthesized. Purity and homogeneity are typically determined using analytical chemistry techniques, for example, polyacrylamide gel electrophoresis, column chromatography, high performance liquid chromatography (HPLC), mass spectrometry analysis, etc. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule is derived, flank the gene. The term therefore includes, for example, a recombinant DNA that is incorporated into a vector, such as an expression vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding one or more additional polypeptide sequences.

By an “isolated polypeptide” is meant a polypeptide or molecule of the disclosure, such as isolated anti-αvβ8 integrin antibody, or an antigen binding fragment thereof, that has been separated from components that naturally accompany it, or from components that are present during an isolation or purification process. Such a polypeptide or molecule is substantially free of other elements present in its natural environment. For instance, an isolated protein is substantially free of cellular material or other proteins from the cell or tissue source from which it is derived. Typically, the polypeptide or molecule is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. The term “isolated” also refers to preparations where the isolated protein or molecule is sufficiently pure to be administered as a pharmaceutical composition, or where the isolated protein or molecule at least 70-80% (w/w) pure, more preferably, at least 80-90% (w/w) pure, even more preferably, 90-95% pure; and, most preferably, at least 95%, 96%, 97%, 98%, 99%, or 100% (w/w) pure. Purity can be measured by any appropriate method, for example, column chromatography, polyacrylamide gel electrophoresis, HPLC analysis and/or by mass spectrometry analysis.

The term “dose” refers to a measured quantity, amount, or concentration of a therapeutic agent, such as a drug, medicine, compound, e.g., a small molecule or biologic, that is administered (without limitation to route of administration) to a subject or patient who has a need for the agent, such as for treatment or therapy benefit.

By “increases” is meant a positive alteration, for example, an increase by at least 10%, 25%, 50%, 75%, 100%, 200%, 300%, 400%, 500%, 1000%, or more.

By “reduces” is meant a negative alteration, for example, a reduction of 10%, 25%, 50%, 75%, or 100%.

By “reference” or “control” is meant a standard of comparison, such as, without limitation, a placebo. In an embodiment, a reference level is the level, expression, or activity of a biomarker in a biological sample obtained from an unaffected tissue.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acid molecules, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “responsive” in the context of therapy is meant susceptible to treatment.

By “specifically binds” or “selectively binds” is meant an agent (e.g., antibody) that recognizes and binds a molecule (e.g., polypeptide, antigen, ligand), but that does not substantially recognize or bind to other molecules in a sample, for example, a biological sample. For example, two molecules (e.g., an antibody and its ligand) that specifically bind to each other form a complex that is relatively stable under physiologic conditions. Specific binding is characterized by a high affinity and a low to moderate capacity, as distinguished from nonspecific binding which usually has a low affinity with a moderate to high capacity.

By “biological sample” or “sample” is meant any liquid, cell, or tissue obtained from a subject. In some embodiments, the biological sample is blood, serum, plasma, cerebrospinal fluid, bronchoalveolar lavage, sputum, tears, saliva, urine, semen, feces, etc. Cell or tissue samples, such as kidney samples, may be further processed in a suitable buffer to produce a homogenate or suspension in which the intracellular components of cells and tissue are provided.

By “subject” is meant a mammal, including, but not limited to, a human, such as a human patient, a human subject, a human individual, a non-human primate, or a non-human mammal, such as a bovine, equine, canine, ovine, or feline animal. In an embodiment, the subject is a human. In an embodiment, a subject is a human patient who has, is at risk for, or who has and is undergoing treatment for a kidney condition or disease, such as CKD, and/or symptoms thereof. The terms “subject,” “individual,” and “patient” may be used interchangeably herein.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

A “pharmaceutical composition” or “formulation” refers to a composition (a physiologically acceptable composition) suitable for pharmaceutical use in a subject, such as an animal or a mammal, including humans. A pharmaceutical composition comprises a therapeutically or prophylactically effective amount of an anti-αvβ8 integrin antibody, or an antigen binding fragment thereof, as described herein and a pharmaceutically acceptable excipient, carrier, vehicle, or diluent. In an embodiment, a pharmaceutical composition encompasses a composition comprising the active ingredient(s) (an anti-αvβ8 integrin antibody, or an antigen binding portion or fragment thereof), and the inert ingredient(s) that constitute the carrier, as well as any product that results, directly or indirectly, from combination, complexation or aggregation of any two or more of the ingredients, or from dissociation of one or more of the ingredients, or from other types of reactions or interactions of one or more of the ingredients. In an embodiment, the pharmaceutical composition optionally includes another biologically active agent, compound, drug, or medicine. Accordingly, the pharmaceutical compositions of the present disclosure embrace any composition that is made by admixing an anti-αvβ8 integrin antibody, or an antigen binding portion or fragment thereof and a pharmaceutically acceptable excipient, carrier, vehicle, or diluent.

A “pharmaceutically acceptable carrier” refers to any of the standard pharmaceutical carriers, buffers, and the like, such as a phosphate buffered saline solution, optionally another biologically active agent, an aqueous (e.g., 5%) solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients, vehicles and diluents may be found in Remington's Pharmaceutical Sciences, 19th Ed. (Mack Publishing Co., Easton, 1995 (or updated editions of this reference)). A pharmaceutical carrier suitable for inclusion in a composition or formulation typically depends upon the intended mode of administration of the active agent, e.g., an anti-αvβ8 integrin antibody as described herein, or an antigen binding portion or fragment thereof. Illustrative modes of administration include enteral (e.g., oral) or parenteral (e.g., subcutaneous, intramuscular, intravenous or intraperitoneal injection; intravenous infusion, or topical, transdermal, or transmucosal administration).

A “pharmaceutically acceptable salt” refers to a salt that can be formulated into a compound for pharmaceutical use, including, but not limited to, metal salts (e.g., sodium, potassium, magnesium, calcium, etc.) and salts of ammonia or organic phosphate.

“Pharmaceutically acceptable,” physiologically acceptable,” or “pharmacologically acceptable” refers to a material that is not biologically, physiological, or otherwise undesirable, i.e., the material may be administered to an individual without causing any undesirable biological effects or without interacting in a deleterious manner with any of the components of the composition in which it is contained or with any components present on or in the body of the individual.

“Physiological conditions” refer to conditions in the body of an animal or mammal, such as a human. Physiological conditions include, but are not limited to, body temperature and an aqueous environment of physiologic ionic strength, pH and enzymes. Physiological conditions also encompass conditions in the body of a particular subject which differ from the “normal” conditions present in the majority of subjects, such as normal human body temperature (approximately 37° C.) or normal human blood pH (approximately 7.4).

As used herein, the terms “treat,” treating,” “treatment,” and the like refer to reducing, diminishing, lessening, alleviating, abrogating, neutralizing, or ameliorating a disorder and/or symptoms associated therewith. It will be appreciated that, although not precluded, treating a disorder or condition does not require that the disorder, condition or symptoms associated therewith be completely eliminated or alleviated. “Treatment” may refer to prophylactic treatment or therapeutic treatment or diagnostic treatment. In certain embodiments, “treatment” refers to the administration of a compound or composition to a subject for therapeutic, prophylactic, or diagnostic purposes.

In accordance with the described methods, treating or treatment involves the administration of an anti-αvβ8 integrin antibody as described herein. In an embodiment, the anti-αvβ8 integrin antibody is administered parentally, e.g., intravenously or subcutaneously, to a subject in need. As will be appreciated by the skilled practitioner in the art, intravenous administration generally refers to providing or delivering an active ingredient, therapeutic agent, substance, medicament, drug, or antibody, such as an anti-αvβ8 integrin antibody, into a vein or blood vessel of a subject to deliver the active ingredient to the systemic circulation of the subject. Intravenous administration may comprise intravenous injection or intravenous infusion into a vein or vessel, e.g., by means of a syringe and needle or catheter. Intravenous injection or infusion may involve the use of plastic tubing and an infusion bag (e.g., an infusion set), such that the active ingredient is delivered through tubing into an infusion bag, and then from the infusion bag into the subject, such as through a catheter and/or a port placed in the subject's body, at a rate of flow that is conventionally and practically determined by a medical practitioner. Intravenous injection or infusion may be carried out with the use of a pump or via a drip.

“Prophylactic treatment” (such as a preventive or protective treatment) is a treatment administered to a subject who does not exhibit signs of a disease, or who exhibits only early signs of the disease, or who is at risk for having a disease, for the purpose of reducing, decreasing, alleviating, or eliminating the risk of developing a disease, pathology, or condition or a more serious or severe form of the disease or pathology, or condition. It is envisioned that the anti-αvβ8 integrin antibodies described herein, or an antigen-binding fragment thereof, or compositions thereof, may be given as a prophylactic or protective treatment to reduce the likelihood of a subject developing a kidney disease, pathology, or condition or to minimize the severity of the kidney disease, pathology, or condition if it develops in the subject.

A “therapeutic” treatment is a treatment administered to a subject who exhibits signs or symptoms of a disease or pathology for the purpose of reducing, diminishing, alleviating, or eliminating the signs or symptoms. The signs or symptoms of disease or pathology may be, without limitation, biochemical, behavioral, cellular, phenotypic, genotypic, histological, functional, physical, subjective, or objective. In an embodiment, an anti-αvβ8 integrin antibody of the disclosure may be given/administered as a therapeutic treatment.

As used herein, a therapeutic that “prevents” a disorder or condition refers to a biologic, a compound or medicinal material that, in a statistical sample, reduces the occurrence of the disorder or condition in the treated sample relative to an untreated control or reference sample, or delays the onset of, or reduces the severity of one or more symptoms of the disorder or condition relative to an untreated reference or control sample. In an embodiment, an anti-αvβ8 integrin antibody of the disclosure is a preventative therapeutic agent in the methods described herein.

The term “effective amount” refers to a dosage sufficient to produce a desired result (e.g., reduction, abatement, elimination, or amelioration of symptoms) related to a health condition, pathology, or disease of a subject or for a diagnostic purpose. The desired result may comprise a subjective or objective improvement in a subject to whom a dose or dosage is administered. “Therapeutically effective amount” refers to that amount of an agent effective to produce the intended beneficial effect on health. It will be understood that the specific dose level and frequency of dosage for any particular patient may depend upon a variety of factors, including the activity of the specific compound employed; the bioavailability, metabolic stability, rate of excretion and length of action of that compound; the mode and time of administration of the compound; the age, body weight, general health, sex, and diet of the patient; and the severity of the patient's particular condition.

The terms “protein”, “peptide” and “polypeptide” refer to chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residues is an artificial chemical mimetic of a corresponding naturally occurring amino acid. Thus, the term “polypeptide” includes full-length, naturally occurring proteins, as well as recombinantly or synthetically produced polypeptides that correspond to a full-length naturally occurring protein or to particular domains or fragments of a naturally occurring protein. The term also encompasses mature proteins which have an added amino-terminal methionine to facilitate expression in prokaryotic cells. Polypeptides can be chemically synthesized or synthesized by recombinant DNA methods; or, they can be purified from tissues in which they are naturally expressed, according to standard biochemical methods of purification. “Functional polypeptides” possess one or more of the biological functions or activities of a given protein or polypeptide, e.g., an anti-αvβ8 integrin antibody. Functional polypeptides may contain a primary amino acid sequence that has been modified from that considered to be the standard sequence of an anti-αvβ8 integrin antibody. Preferably, such modifications are conservative amino acid substitutions that do not alter or substantially alter the normal function or activity of the protein. A polypeptide fragment, portion, or segment refers to a stretch of amino acid residues of at least about 6 contiguous amino acids from a particular sequence, more typically at least about 10-12 contiguous amino acids.

Nucleic acid molecules (polynucleotides), which encode polypeptides such as an anti-αvβ8 integrin antibody of the present disclosure, include any nucleic acid molecule that encodes the disclosed polypeptide, e.g., an anti-αvβ8 integrin antibody, or an antigen-binding fragment thereof. Such nucleic acid molecules need not be 100% identical to an endogenous nucleic acid sequence, but will typically exhibit substantial identity. Polynucleotides having “substantial identity” to an endogenous sequence are typically capable of hybridizing with at least one strand of a double-stranded nucleic acid molecule. Polynucleotides having “substantial identity” to an endogenous sequence are typically capable of hybridizing with at least one strand of a double-stranded nucleic acid molecule. By “hybridize” is meant pairing to form a double-stranded molecule between complementary polynucleotide sequences (e.g., a gene), or fragments thereof, under various conditions of stringency. (See, e.g., Wahl, G. M. and S. L. Berger, 1987, Methods Enzymol., 152:399; Kimmel, A. R., 1987, Methods Enzymol., 152:507).

By way of nonlimiting example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a particular embodiment, hybridization occurs at 30° C. in 750 mM NaCl, 75 mM trisodium citrate, and 1% SDS. In another particular embodiment, hybridization occurs at 37° C. in 500 mM NaCl, 50 mM trisodium citrate, 1% SDS, 35% formamide, and 100 μg/ml denatured salmon sperm DNA. In another particular embodiment, hybridization occurs at 42° C. in 250 mM NaCl, 25 mM trisodium citrate, 1% SDS, 50% formamide, and 200 μg/ml salmon sperm DNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, and, in particular, less than about 15 mM NaCl and 1.5 mM trisodium citrate. Stringent temperature conditions for the wash steps will ordinarily include a temperature of at least about 25° C., or at least about 42° C., or at least about 68° C. In a particular embodiment, wash steps will occur at 25° C. in 30 mM NaCl, 3 mM trisodium citrate, and 0.1% SDS. In another particular embodiment, wash steps will occur at 42 C in 15 mM NaCl, 1.5 mM trisodium citrate, and 0.1% SDS. In another particular embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science, 196:180, 1977); Grunstein and Hogness (Proc. Natl. Acad. Sci., USA, 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences (see e.g., Karlin et al., 1990, Proc. Natl. Acad. Sci., 87:2264-2268, as modified in Karlin et al., 1993, Proc. Natl. Acad. Sci., 90:5873-5877, and incorporated into the NBLAST and)(BLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR).

By “substantially identical” is meant a polypeptide or nucleic acid molecule exhibiting at least 50% identity to a reference amino acid sequence or nucleic acid sequence. Such a sequence may be at least 60%, or at least 80% or 85%, or at least 90%, 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following amino acid groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

A mammalian anti-αvβ8 integrin antibody (particularly, an anti-human αvβ8 integrin antibody), or an immunologically functional allelic variant or an isoform thereof, may be useful in the described methods, as are other variants or isoforms, including fragments of the antibody that possess the binding and blocking activity of the anti-αvβ8 integrin antibody. An “allelic variation” in the context of a polynucleotide or a gene is an alternative form (allele) of a gene that exists in more than one form in the population. At the polypeptide level, “allelic variants” generally differ from one another by only one, or at most, a few amino acid substitutions. A “species variation” of a polynucleotide or a polypeptide is one in which the variation is naturally occurring among different species of an organism.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from its context, the term “or” as used herein is understood to be inclusive. Unless specifically stated or obvious from context, the terms “a”, “an”, and “the” as used herein are understood to be singular or plural.

Unless specifically stated or obvious from context, as used herein, the term “about” is understood as within a range of normal tolerance in the art, for example within 2 standard deviations of the mean. The term “about” is understood to refer to within 5%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS/FIGURES

FIGS. 1A and 1B present graphs and tables showing the binding of specific anti-αvβ8 integrin antibodies to αvβ8 integrin protein as disclosed herein. More specifically, FIG. 1A shows a graph depicting a comparison of the binding affinity between IgG anti-αvβ8 integrin antibody “hu37E1B5” produced using a sequence disclosed in WO 2013/026004, and a chimeric IgG anti-αvβ8 integrin antibody “Chi-37E1B5” as described herein in Example 1. As observed from the binding results shown in FIG. 1A, the hu37E1B5 anti-αvβ8 integrin antibody has very poor binding affinity compared with that of the Chi-37E1B5 anti-αvβ8 integrin antibody. FIG. 1B presents a graph showing a comparison of the affinities of different IgG anti-αvβ8 integrin antibodies (“hu37E1B5” and “Chi-37E1B5” as discussed in FIG. 1A, and a CDR-grafted antibody called “MEDI-hu37E1B5”) for binding to αvβ8 integrin protein, and a table of the K_{d} measurements of these antibodies, as assessed by Biacore assay. The MEDI-hu37E1B5 anti-αvβ8 integrin antibody was generated using CDR grafting from anti-αvβ8 integrin antibody Chi-37E1B5 and showed an αvβ8 integrin binding profile that was similar to that of the Chi-37E1B5 anti-αvβ8 integrin antibody (FIG. 1B).

FIGS. 2A-2D present the results and amino acid sequences of representative anti-αvβ8 integrin antibody clonal hits from the generation of saturation point mutations in the CDR positions of the MEDI-hu37E1B5 humanized anti-αvβ8 integrin antibody C94I, along with graphs showing the binding affinity analyses of the MEDI-hu37E1B5 C94I anti-αvβ8 integrin antibody and representative anti-αvβ8 integrin V_{H}CDR1, V_{H}CDR3 and V_{L} hits, called “P1” or “P2,” as generated by saturation point mutation experiments and identified in the screening analysis described in Example 1. FIG. 2A shows the improved binding affinity of the V_{H}CDR1 hits to αvβ8 integrin compared with that of the MEDI-hu37E1B5 parental antibody. FIG. 2B shows the improved binding affinity of the V_{H}CDR3 hits to αvβ8 integrin compared with that of the MEDI-hu37E1B5 parental antibody. FIG. 2C shows the improved binding affinity of the V_{L} hits to αvβ8 integrin compared with that of the MEDI-hu37E1B5 parental antibody. FIG. 2D presents alignments of the amino acid sequences of the V_{H} and V_{L} regions of representative primary clonal anti-αvβ8 integrin antibody hits, designated “P2-23,” “P2-33,” “P2-25,” “P1-21,” “P1-35,” “P1-42,” “P2-16,” “P2-19,” “P2-36,” and “P2-14,” obtained from the screening of affinity matured anti-αvβ8 integrin antibody clones. The framework (FW1-FW4) regions and CDRs (CDR1-CDR3) in the V_{H} and V_{L} regions of the clones are designated above the sequences. Differences in the amino acid residues in the CDR regions are indicated by double underlining.

FIGS. 3A and 3B present αvβ8 integrin binding data from the combination library screening used to generate the humanized and affinity optimized anti-αvβ8 antibody as described in Example 1. As represented in FIGS. 3A and 3B, all 10 beneficial point mutations were combined in a combinatorial fashion. 4608 clones were screened and 88 clones were selected for confirmation. 6 combo hits were identified which showed additive binding improvement over the best primary hit P2-23.

FIG. 4 presents the results of an enzyme linked immunosorbent assay (ELISA) in which different concentrations of humanized MEDI-hu37E1B5, affinity optimized B5-15 and B5-15 N59Q anti-αvβ8 integrin antibodies were compared for binding to αvβ8 integrin protein. For the ELISA assays, recombinantly produced αvβ8 integrin protein was coated onto the wells of a tissue culture plate. Antibody binding was detected using a horse radish peroxidase (HRP)-conjugated goat anti-human Fc antibody. Improved binding of the affinity optimized B5-15 and B5-15 N59Q anti-αvβ8 integrin antibodies compared to that of the parent MEDI-hu37E1B5 anti-αvβ8 integrin antibody was observed over a range of antibody concentrations. B5-15 N59Q is an aglycosylated version of the B5-15 anti-αvβ8 integrin antibody. Glycosylation of anti-αvβ8 integrin antibodies (in the HCDR2 sequence) has been shown to be important for inhibitory activity but does not affect binding to αvβ8 integrin (see WO 2015/195835).

FIG. 5 presents a graph and table showing the results of a TMLC luciferase bioassay to measure the inhibition of anti-αvβ8 integrin antibodies on TGF-β activation. The graph in FIG. 5 shows the percent maximal response of TGF-β activity versus anti-αvβ8 integrin antibody (IgG isotype). The anti-αvβ8 integrin antibodies assessed in the assay were the parental (Chi-37E1B5, shown as “Chi-B5” in the figure) and affinity optimized (B5-15) anti-αvβ8 integrin antibodies. The K_{d} (pM) and IC₅₀ (nM) values are shown for the two antibodies in the table below the graph. As observed from the graph, an increase in concentration of the anti-αvβ8 integrin antibodies in the assay resulted in decreased TGF-β activation, with B5-15 demonstrating a greater in vitro potency than Chi-37E1B5.

FIG. 6 presents an alignment of the amino acid sequences of the V_{H} and V_{L} regions of four anti-αvβ8 integrin antibodies, “Chi-37E1B5” (the chimeric anti-αvβ8 integrin antibody in-licensed from UCSF), “hu37E1B5” (the UCSF humanized 37E1B5 antibody from WO 2013/026004), “MEDI-hu37E1B5” (the MedI humanized anti-αvβ8 integrin antibody) and “B5-15” (the humanized and affinity optimized B5-15 anti-αvβ8 integrin antibody). Differences in the amino acid sequence from “Chi-37E1B5” are highlighted in bold. The V_{H} and V_{L} CDRs are underlined in each variable region sequence.

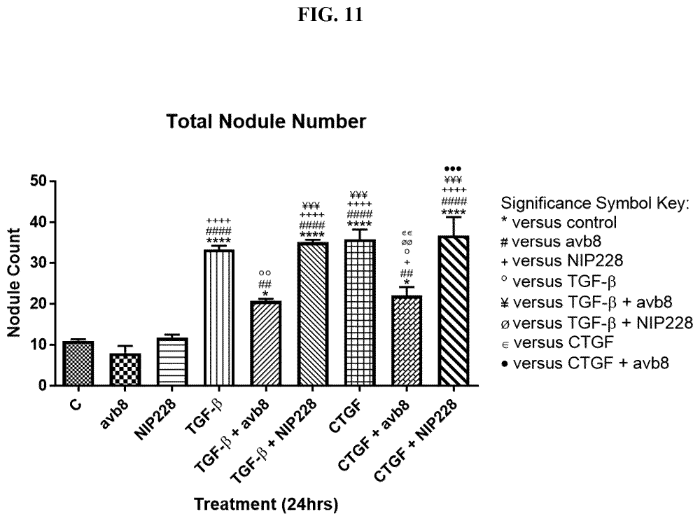

As shown in FIG. 6, the amino acid sequence of the V_{H} region of the Chi-37E1B5 anti-αvβ8 integrin antibody is as follows:

(SEQ ID NO: 20)
EVQLVESGGGLVQPGGSLNLSCAVSGFVFSRYWMSWVRQAPGKGLEWIGEI
NPDSSTINYTSSLKDKFIISRDNAKNTLYLQMNKVRSEDTALYYCACLITT
EDYWGQGTSVTVSS.

The nucleotide sequence of the V_{H} region of the Chi-37E1B5 antibody is as follows:

(SEQ ID NO: 21)
gaagtgcagctggtggagtctggaggtggcctggtgcagcctggaggatcc
ctgaacctctcctgtgcagtctcaggattcgtttttagtagatactggatg
agttgggtccggcaggctccagggaaagggctagaatggattggagaaatt
aatccagatagcagtacgataaactatacgtcatctctaaaggataaattc
atcatctccagagacaacgccaaaaatacgttgtacctgcaaatgaacaaa
gtgagatctgaggacacagccctttattactgtgcatgtcttattactacg
gaggactactggggtcaaggaacctcagtcaccgtctcctca.

The amino acid sequence of the V_{L} (kappa) region of the Chi-37E1B5 anti-αvβ8 integrin antibody is as follows:

(SEQ ID NO: 22)
EIVLTQSPSSMYASLGERVTIPCKASQDINSYLSWFQQKPGKSPKTLIYYA
NRLVDGVPSRFSGSGSGQDYSLTISSLEYEDMGIYYCLQYDEFPYTFGGGT
KLEIK.

The nucleotide sequence of the V_{L} (kappa) region of the Chi-37E1B5 antibody is as follows:

(SEQ ID NO: 23)
gaaattgtgctgactcagtctccatcttccatgtatgcatctctaggagag
agagtcactatcccttgcaaggcgagtcaggacattaatagctatttaagc
tggttccagcagaaaccagggaaatctcctaagaccctgatctattatgca
aacagattggtagatggggtcccatcaaggttcagtggcagtggatctggg
caagattattctctcaccatcagcagcctggagtatgaagatatgggaatt
tattattgtctacagtatgatgagtttccgtacacgttcggaggaggcacc
aagctggaaatcaaa.

Also in FIG. 6, the amino acid sequence of the V_{H} region of the UCSF hu37E1B5 anti-αvβ8 integrin antibody is as follows:

(SEQ ID NO: 24)
EVQLVESGGGLVQPGGSLRLSCAVSGFVFSRYWMSWVRQAPGKGLEWIGEI
NPDSSTINYTSSLKDRFTISRDNAKNSLYLQMNSLRAEDTAVYYCASLITT
EDYWGQGTTVTVSS.

The amino acid sequence of the V_{L} (kappa) region of the UCSF hu37E1B5 antibody is as follows:

(SEQ ID NO: 25)
EIVLTQSPSSLSLSPGERVTITCKASQDINSYLSWYQQKPGKAPKLLIYYA
NRLVDGVPARFSGSGSGQDYTLTISSLEPEDFAVYYCLQYDEFPYTFGGGT
KLEIK.

FIGS. 7A-7C show photomicrograph images of human kidney tissues stained by immunohistochemistry (IHC) with an anti-αvβ8 integrin antibody. As shown in FIG. 7A, human kidney tissue was found to be highly enriched in αvβ8 integrin, particularly in the podocytes compared with other healthy human tissues evaluated, except for nerve tissue. FIG. 7B shows that αvβ8 integrin is abundant in kidney tissue samples obtained from individuals with diabetic nephropathy (DN) and chronic kidney disease (CKD), based on the pattern of staining with the αvβ8 integrin antibody. In particular, in the kidney tissue samples obtained from the DN and CKD patients, αvβ8 integrin staining was essentially found in tubules. The glomeruli of kidneys in DN patients showed decreased αvβ8 integrin staining, likely as a consequence of podocyte loss due to kidney tissue fibrosis and damage. FIG. 7C shows the results of IHC staining with an anti-αvβ8 integrin antibody of kidney tissues from normal individuals (“normal kidney”) and kidney tissues from patients who have different stages of diabetic nephropathy (“DN”). The results show that αvβ8 staining was elevated in viable functional nephrons. In kidney tissue samples obtained from patients having DN, the unstained areas are the fibrotic matrix that replaced functional nephrons and are designated by an asterisk (*).

FIGS. 8A-8E present bar graphs, dot plot and box plot graphs showing results from the transcriptomic prolife analysis performed using kidney tissue samples obtained from patients who had diabetic nephropathy (DN) kidney disease compared with kidney tissue samples obtained from living donors. FIG. 8A shows the relative mRNA expression levels (relative to hprt1 expression) of different AV associated integrins, ITGB8, ITGB1, ITGB3, ITGB5 and ITGB6 in kidney tissue obtained from human subjects having CKD. ITGB8 is the most abundant (38 subunit in kidneys of CKD patients. FIG. 8B presents a box plot graph showing that ITGB8 mRNA expression normalized to NPHS1 (nephrin, a podocyte specific gene) mRNA was higher in the glomeruli of DN patient kidney samples relative to its expression in living donors as healthy controls. FIG. 8C presents box plot graphs showing that ITGB8 mRNA expression normalized to NHPS1 mRNA was higher in the tubule-interstitium of DN patient kidney samples relative to its expression in living donors as healthy controls. FIG. 8D presents a dot plot graph showing that ITGB8 mRNA expression was strongly correlated with the TGF-β activation score (a composite of downstream genes in the TGF-β pathway) across CKD in the tubule-interstitium of patients with CKD. FIG. 8E presents a box plot graph showing the mRNA expression levels (normalized counts) of the different integrin genes (ITGAV, ITGB2, ITGB4, ITGB5, ITGB6, ITGB7 and ITGB8) in healthy donor kidney glomerulus (Glomeruli-LD), in kidney glomerulus from patients having DN (Glomeruli-DN), in kidney tubule-interstitium from healthy donors (Tub-LD) and in kidney tubule-interstitium from patients having DN (Tub-DN) following whole genome transcriptional profiling using RNAseq. Increased ITGB8 mRNA expression in the tubule-interstitium of DN patients (n=20) versus living donors (LD, n=19) was found (p<0.01).

FIGS. 9A-9J present photomicrograph images showing results from IHC staining using an anti-αvβ8 integrin antibody as described herein (FIGS. 9A-9D) and bar graphs showing the results of in vivo analyses of mRNA expression (FIGS. 9E-9I) and percent hydroxyproline content as an indicator of fibrosis (FIG. 9J) in humanized αvβ8 transgenic mice that had undergone a unilateral ureteral occlusion (UUO) procedure (a mouse model of kidney fibrosis).

The IHC staining photomicrographs shown in FIGS. 9A and 9B demonstrate that humanized αvβ8 transgenic mice express αvβ8 mainly in the glomerulus of the kidney, similar to what is typically observed in healthy human kidney. The induction of fibrosis with the UUO procedure was demonstrated to increase αvβ8 expression in the kidney tubules (FIGS. 9C and 9D), similar to what is typically observed in the kidneys of humans having CKD.

As shown in FIG. 9E, the anti-αvβ8 integrin antibodies Chi-37E1B5 (labelled as Parental Avb8 Ab) and B5-15 (labelled as Lead Avb8 Ab) attenuated UUO-induced increases in collagen 1a1 mRNA expression at 8-days post-UUO surgery relative to UUO controls. 10 mg/kg of each of the antibodies was administered.

As shown in FIG. 9F, the anti-αvβ8 integrin antibodies Chi-37E1B5 (labelled as Parental Avb8 Ab) and B5-15 (labelled as Lead Avb8 Ab) attenuated UUO-induced increases in collagen 3a1 mRNA expression at 8-days post-UUO surgery relative to UUO controls. 10 mg/kg of each of the antibodies was administered.

As shown in FIG. 9G, UUO increased obstructed kidney cortical fibronectin 1 (Fn1) mRNA expression at 8-days post-UUO surgery relative to sham controls. Antibodies Chi-37E1B5 (labelled as Parental Avb8 Ab) and B5-15 (labelled as Lead Avb8 Ab) attenuated UUO-induced increases in Fn1 expression at 8-days of injury duration compared to UUO controls. 10 mg/kg of each of the antibodies was administered.

As shown in FIG. 9H, the anti-αvβ8 integrin antibody B5-15 (labelled as Lead Avb8 Ab) attenuated a UUO-induced increase in α-smooth muscle actin (α-SMA) expression at 8-days post-UUO surgery relative to UUO controls. The Chi-37E1B5 (labelled as Parental Avb8 Ab) did not reduce the UUO-induced increase in α-SMA. 10 mg/kg of each of the antibodies was administered.

As shown in FIG. 9I, the anti-αvβ8 integrin antibodies Chi-37E1B5 (labelled as Parental Avb8 Ab) and B5-15 (labelled as Lead Avb8 Ab) attenuated UUO-induced increases in connective tissue growth factor (CTGF) mRNA expression at 8-days post-UUO surgery relative to UUO controls. 10 mg/kg of each of the antibodies was administered.

As shown in FIG. 9J, UUO increased obstructed kidney cortical % hydroxyproline (OH—P) at 8-days post-UUO surgery. The Chi-37E1B5 (labelled as Parental Avb8 Ab) and B5-15 (labelled as Lead Avb8 Ab) antibodies attenuated UUO-induced increases in % OH—P at 8-days UUO injury duration compared to controls. 10 mg/kg of each of the antibodies was administered. Renal cortical hydroxyproline readout serves as a measurement of actual fibrotic content/fibrosis of tissue.

FIGS. 10A and 10B show graphs related to the effects downstream of TGF-β signaling in humanized αvβ8 transgenic animals having UUO surgery following treatment with an isotype control antibody (NIP228) or an anti-αvβ8 integrin antibody (B5-15). FIG. 10A shows that UUO surgery in humanized αvβ8 transgenic mice resulted in an increase in TGF-β-dependent SMAD2/3 phosphorylation by 5.7-fold versus the Sham-treated group. Treatment with the anti-αvβ8 integrin antibody (B5-15) significantly diminished SMAD2/3 activation by 1.6-fold compared to treatment with the isotype control. Total levels of SMAD2/3 were increased in all UUO groups compared to Sham treated animals (FIG. 10B).

FIG. 11 shows a graph demonstrating the effect of treatment of a renal primary tri-culture cell system with either B5-15 (an anti-αvβ8 integrin antibody) or NIP228 (an isotype control). This tri-culture cell system is a model of human glomerulosclerosis where glomerular endothelial cells, podocytes, and mesangial cells form a vascular network (Waters et al., 2017, J Pathol, 243(3):390-400). Treatment with TGF-β or CTGF induces formation of nodules, an indicator of fibrosis. Treatment with an anti-αvβ8 integrin antibody significantly reduces nodule number in comparison to treatment with an isotype control.

### DETAILED DESCRIPTION

The present disclosure generally features antibodies, compositions and methods for treating kidney disease, e.g., diabetic nephropathy (DN), chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like, in an individual in need as described herein. In particular, the antibodies, compositions and methods are directed to treating kidney fibrosis, which is associated with kidney disease, such as CKD.

The present disclosure is directed to a treatment method for ameliorating, attenuating, abrogating, reducing, or alleviating fibrosis in kidney tissue in a subject having kidney disease, such as CKD. In general, fibrosis refers to the formation of excess fibrous connective tissue (scar tissue) in an organ such as the kidney, which causes thickening and scarring of the kidney connective tissue. As noted supra, the methods involve the administration of an anti-αvβ8 integrin antibody, or an antigen binding fragment thereof, which specifically binds to αvβ8 integrin found to be highly expressed on diseased kidney cells and in kidney tissue, particularly, kidney epithelial cells and tissue in subjects having kidney disease, such as CKD. The anti-αvβ8 integrin antibodies, or an antigen binding fragment thereof, selectively bind to αvβ8 integrin on fibrotic kidney cells and tissues, thereby blocking, neutralizing, or inhibiting the interaction of the kidney-expressed αvβ8 integrin with the latent form of TGF-β (LAP-TGF-β) at the kidney cell surface. The anti-αvβ8 integrin antibody binding interferes with the αvβ8 integrin/LAP TGF-β interaction, which, in turn, blocks or prevents the activation of TGF-β at the kidney cell surface, so that active TGF-β is not produced and thus cannot exert its cellular effects associated with kidney fibrosis in the kidney tissue of a subject, such as a human or a non-human subject. The methods provide therapeutic benefit, particularly in the treatment of kidney disease, for example, by reducing, attenuating, abrogating, or decreasing the damaging fibrosis induced by active TGF-β in kidney disease, e.g., CKD.

Without wishing to be bound by a particular theory, the anti-αvβ8 integrin antibody, or an antigen-binding fragment thereof, reduces local TGF-β activation in kidney cells and tissue where αvβ8 integrin is highly expressed, for example, by directly binding to the αvβ8 integrin receptor of LAP TGF-β. The binding of an anti-αvβ8 integrin antibody to αvβ8 integrin, which blocks the activation of TGF-β from its latent form, may also reduce or prevent recruitment of the protease that cleaves latent TGF-β and releases the mature, active TGF-β peptide. This could occur by the anti-αvβ8 integrin antibody inhibiting the binding of αvβ8 integrin on the kidney cell surface to latent TGF-β associated with the cell matrix, thereby inhibiting the subsequent activation of TGF-β as described infra.

Transforming Growth Factor Beta (n), (TGF-β) and its Interaction with αvβ8 Integrin

In cells, the TGF-β cytokine is synthesized and secreted to the extracellular matrix as an inactive precursor that is complexed to a “latency-associated peptide (LAP)” and a “latent TGFβ binding protein (LTBP).” The latent form of TGF-β must be activated in order to bind to its receptor, e.g., αvβ8 integrin, and have biological function (J. J. Worthington et al., 2011a, Trends Biochem. Sci., 36:47-54). The LAP is cleaved from the active TGF-β, but remains non-covalently attached in a conformation that prevents TGF-β from engaging its receptor. Activators of TGF-β include a variety of proteases and cell surface molecules that alter the latent complex allowing active TGF-β to engage its receptor. Putative TGF-β activators include, without limitation, proteases that degrade LAP, thrombospondin-1, reactive oxygen species (ROS) and integrins. Activation of the latent complex is thus essential for the regulation of TGF-β function, and TGF-β activators are the rate-limiting step in the conversion of latent to active TGF-β. By way of example, in human CKD kidneys (n=4), the amount of latent TGF-β is 53-fold higher than that of active TGF-β.

Fibrosis is an important driver of chronic kidney disease (CKD) progression in human patients and correlates with renal dysfunction and damage. TGF-β is involved in the development of renal fibrosis in CKD. Renal TGF-β is upregulated in human fibrotic CKD versus control kidney (D. S. Goumenos et al., 2002, Nephrol. Dial. Transplant., 17:2145-2152). Urinary TGF-β was shown to correlate with renal damage (albuminuria) in Type 2 diabetes. (Marwood et al., 2002, Exp. Biol. Med., 227(11):943-956).

The αv-integrin transmembrane receptors, e.g., αvβ8, are important players in the regulation of extracellular matrix physiology and in the activation of TGF-β. Briefly, αv-integrins mediate activation of latent-TGF-β. In particular, αvβ8 binds to the RGD (arginine-glycine-aspartic acid) motif of the TGF-β-binding latency-associated peptide (LAP), thereby regulating the levels of free and active TGF-β in tissues (Mu, D. et al., 2002, J. Cell Biol., 157(3):493-507; Araya, J., 2006, Am. J. Pathol., 169(2):405-415).

Unlike the activity of other αvβ integrins, αvβ8 integrin is constitutively active, and the activation of LAP TGF-β (by release of active TGF-β cytokine after binding of LAP TGF-β to αvβ8 integrin) is mediated by cleavage by the MMP-14 protease, rather than by anchoring to cytoplasmic actin (no traction effect). αvβ8 integrin expression is enriched in kidney tissue, and the gene encoding αvβ8 integrin is highly expressed in kidney tissue compared with other tissues, such as, for example, pancreas, liver, gallbladder, salivary gland, esophagus, stomach, intestine, lung, heart, or bladder, as exemplified infra.

As described herein, both in vitro and in vivo studies have demonstrated that high levels of expression of αvβ8 integrin on kidney epithelial cells directly correlated with high levels of kidney tissue fibrosis resulting from the activation of TGF-β. High levels of TGF-β activity induce and increase damage to renal (kidney) cells and tissue, causing fibrosis, and thus seriously exacerbate kidney disease, such as CKD. The anti-αvβ8 integrin antibodies described herein specifically bind to αvβ8 integrin expressed by kidney cells and inhibit TGF-β's destructive activity and consequent fibrosis in kidney cells and tissue by blocking and/or reducing αvβ8 integrin's binding to latent TGF-β (LAP TGF-β) and inhibiting release of the active form of TGF-β. This action of the specific anti-αvβ8 integrin antibodies serves as a treatment against kidney cell and tissue destruction resulting from TGF-β activity, e.g., by inhibiting TGF-β's intracellular signaling cascade. In accordance with the methods disclosed and exemplified herein, blocking αvβ8 integrin activity by providing antibodies that specifically bind αvβ8 integrin in the kidney significantly reduce activation of TGF-β localized in kidney and thereby specifically reducing kidney cell and tissue damage, namely, kidney fibrosis, in diseased kidneys.

The use of anti-αvβ8 integrin antibodies that specifically target the αvβ8 integrin receptor on kidney cells stemmed from the discoveries, as described herein, that αvβ8 integrin is preferentially expressed in the kidney in normal subjects and that the expression of αvβ8 integrin is significantly increased and localized in kidney epithelial cells (e.g., podocytes and interstitial tubules) in the kidneys of subjects with fibrotic kidney disease, e.g., human patients with CKD. As noted supra, the present disclosure provides surprising findings that the αvβ8 protein is highly up-regulated in kidneys of human patients with CKD. Moreover, the activation of TGF-β by the specific binding of αvβ8 integrin to the latent active form of TGF-β in the kidney is a direct cause of destructive fibrosis in kidney tissue. The present discoveries are contradictory to a prior finding in the art that αvβ8 integrin is found primarily in mesangial cells of the kidney and that transgenic animals which did not express mesangial cell αvβ8 integrin nevertheless harbored active TGF-β that caused endothelial cell apoptosis (S. Khan et al., 2011, Am. J. Pathology, 178(2):609-620). By contrast and as described and exemplified herein, the present methods involve the inhibition and blockage of αvβ8 integrin's interaction with and binding to LAP-TGF-β, such that active TGF-β is not released at the kidney cell membrane and is not able to cause fibrosis (and/or further damage) to kidney cells and tissue in subjects afflicted with kidney disease such as CKD.

Antibodies Specifically Directed Against αvβ8 Integrin

The present disclosure encompasses the development and use of antibodies that are directed against and specifically target and bind to αvβ8 integrin, particularly, to αvβ8 integrin expressed in kidney cells and tissue and in fibrotic kidney cells and tissue. These antibodies, or antigen binding fragments thereof, are of great benefit in methods of treating kidney disease, particularly, kidney fibrosis in kidney disease, such as chronic kidney disease (CKD), in a subject in need of treatment. In embodiments, the subject may have a condition that is associated with damage or injury to kidney cells and tissue and that causes fibrosis of kidney tissue as described herein, or an acute, chronic, or end stage kidney disease. Treatment of subjects having kidney fibrosis and kidney disease involving fibrosis, regardless of the etiology, using the antibodies, compositions and methods described herein provides an important medical and clinical benefit to subjects in need, especially patients afflicted with kidney disease, such as CKD or DN. In an embodiment, the anti-αvβ8 integrin antibody is a humanized antibody.

In one embodiment, the anti-αvβ8 integrin antibody is a humanized antibody, referred to as “MEDI-hu37E1B5” antibody as described supra, which specifically targets and binds to human αvβ8 integrin. In a particular embodiment, the MEDI-hu37E1B5 antibody specifically targets and binds to human αvβ8 integrin that is expressed in the kidney and that is highly expressed in fibrotic kidney. In an embodiment, the MEDI-hu37E1B5 antibody does not cross-react with antibodies against other integrins.

In another embodiment, the anti-αvβ8 integrin antibody is a humanized and affinity optimized antibody, referred to as “B5-15” anti-αvβ8 integrin antibody as described supra, which specifically targets and demonstrates high affinity binding to the human αvβ8 integrin. The optimized B5-15 antibody is of the IgG1 subtype, demonstrates specific and selective binding to human αvβ8 integrin and exhibits functional activity by blocking or inhibiting the binding interaction or association between human αvβ8 integrin with TGF-β latent form, thus blocking or inhibiting the activation of TGF-β by release of active TGF-β from its latent form. As demonstrated herein (FIG. 4), B5-15 has an improved profile for binding to αvβ8 integrin compared with the CDR-grafted MEDI-hu37E1B5 anti-αvβ8 integrin antibody described in Example 1.

The B5-15 antibody blocks the binding of αvβ8 integrin to LAP-TGF-β and blocks the activation of TGF-β and intracellular signaling by TGF-β, thus protecting kidney cells and tissue from the damaging effects of the active TGF-β peptide, which can induce and exacerbate fibrosis. Without wishing to be bound by theory, the B5-15 antibody allosterically modifies the αvβ8 integrin and reduces its affinity for the latent TGF-β (LAP) binding domain, which prevents the activation of TGF-β from its latent form so that no active TGF-β peptide is released. Thus, the antibody induces a conformational change in αvβ8 integrin, such that αvβ8 can no longer bind to latent TGF-β to facilitate its activation (WO 2015/195835). Until the present disclosure, the binding properties and functional activities of anti-αvβ8 integrin antibodies, such as the B5-15 antibody, in renal (kidney) fibrosis were unknown.

In embodiments, the anti-αvβ8 integrin antibodies disclosed herein specifically bind to the αvβ8 integrin receptor that has elevated expression on kidney cells and tissue, particularly diseased, damaged, and/or fibrotic kidney tissue such as is found in individuals with kidney disease, e.g., CKD or DN. Compositions comprising these antibodies and their use in methods of treating kidney disease and nephropathy, particularly, kidney disease involving fibrosis, are encompassed by the present disclosure. The described antibodies bind only to human αvβ8 integrin and do not cross-react with any other integrins.

The described antibodies are also advantageous because they selectively target and specifically bind to the αvβ8 integrin receptor for latent TGF-β and do not directly target the cytokine itself, thus providing a safer therapeutic approach for treating kidney disease, particularly, kidney disease involving fibrosis. In addition, the inhibition, blocking, or neutralization of the activity of the TGF-β1 isoform is especially advantageous, as this TGF-β isoform is generally considered to account for the majority of the disease-related activity of TGF-β. The prevalence of the TGF-β1 isoform in kidney is likely to result in the involvement of the active form of TGF-β1 in kidney fibrosis and kidney disease.

While targeting TGF-β directly may be one approach for inhibiting or preventing pathologies caused by TGF-β activity, a general neutralization and/or chronic inhibition of the actions of TGF-β resulting from directly targeting the cytokine could have grave side effects in the treated individual, given the involvement of TGF-β in modulating diverse cellular functions and pathways. Thus, the approach of using anti-αvβ8 integrin antibodies as provided herein to block, inhibit, neutralize, and thus effectively prevent, the αvβ8 integrin/LAP TGF-β interaction on kidney cells and tissue without compromising in vivo TGF-β activation in other cells, tissues and organs, or for other physiological purposes, provides a valuable therapeutic tool and method for treating kidney disease and fibrosis, such as CKD or DN. Advantageously, the specificity of the anti-αvβ8 antibodies described herein for kidney cells and tissue expressing high levels of the αvβ8 integrin decreases adverse effects, such as autoimmune responses, rapid-onset atherosclerosis and carcinoma development. Adverse effects have been seen with pan-TGF-β inhibition, therefore specifically targeting αvβ8 integrin to affect TGF-β activation is likely to result in reduced adverse events.

As another advantage, the anti-αvβ8 integrin antibodies described herein do not cross the blood-brain-barrier (BBB) and thus cannot result in binding to αvβ8 integrin expressed on cells and tissue of the brain.

The described anti-αvβ8 antibodies specifically block the binding of kidney epithelial cell-expressed αvβ8 integrin to the latent form of TGF-β and thus block fibrosis caused by the release in kidney tissue of active TGF-β, which has been found to play a central role in the glomerular and tubule-interstitial pathobiology of renal disease that induce alterations of glomerular filtration barrier, glomerulosclerosis and fibrosis, as well as the degeneration of tubules leading to permanent renal dysfunction. Accordingly, the present methods involve the use of specific anti-αvβ8 integrin antibodies to treat kidney disease, such as chronic kidney disease or diabetic nephropathy characterized by deleterious kidney tissue fibrosis, by specifically binding to a target receptor, i.e., αvβ8 integrin, that is highly expressed on the surface of kidney cells in individuals having damaged kidneys and/or kidney disease, rather than targeting TGF-β itself. The targeting and binding of αvβ8 integrin by the specific anti-αvβ8 integrin antibodies provided herein abrogates and effectively prevents TGF-β cytokine activity that is a major culprit in causing kidney tissue fibrosis and further damage to kidney tissue in kidney disease.

The anti-αvβ8 integrin antibodies described herein specifically bind to one or more regions of the αvβ8 integrin receptor protein that contain antigen binding sites or epitopes. In an embodiment, the epitope of αvβ8 integrin bound by the anti-αvβ8 integrin antibody, such as the MEDI-hu37E1B5 antibody or the B5-15 antibody was mapped to a region approximately 28A (Angstroms) away from the αvβ8 integrin and LAP-TGF-β binding site. (S. Minagawa et al., 2014, Sci. Transl. Med., 6(241): 241re79. Doi:10.1126/scitranslmed.3008074).

In an embodiment, an antibody that competes for binding to αvβ8 integrin with an antibody having a light chain variable region comprising the following three light chain CDRs: V_{L} CDR1: KASQDINSYLS (SEQ ID NO: 4); V_{L} CDR2: YANRLVD (SEQ ID NO: 5); and V_{L} CDR3: LQYDEFPYT (SEQ ID NO: 6); and a heavy chain variable region comprising the following three heavy chain CDRs: V_{H} CDR1: RYWMS (SEQ ID NO: 1); V_{H} CDR2: EINPDSSTINYTSSL (SEQ ID NO: 2); and V_{H} CDR3: LITTEDY (SEQ ID NO: 3) as described herein is contemplated. The antibody can be monoclonal, chimeric, humanized, etc., and can be of isotype IgG1, IgG2, IgG2a, IgG3 or IgG4. In a particular embodiment, the antibody is an IgG1 antibody. In some embodiments, the antibody is a human, humanized, or chimeric antibody.

In another embodiment, an antibody that competes for binding to αvβ8 integrin with an antibody having a light chain variable region comprising the following three light chain CDRs: V_{L} CDR1: KASQDINKYLS (SEQ ID NO: 10); V_{L} CDR2: YANRLVD (SEQ ID NO: 5); and V_{L} CDR3: LQYDVFPYT (SEQ ID NO: 11); and a heavy chain variable region comprising the following three heavy chain CDRs: V_{H} CDR1: RSWIS (SEQ ID NO: 9); V_{H} CDR2: EINPDSSTINYTSSL (SEQ ID NO: 2); and V_{H} CDR3: LITTEDY (SEQ ID NO: 3) as described herein is contemplated. The antibody can be monoclonal, chimeric, humanized, etc., and can be of isotype IgG1, IgG2, IgG2a, IgG3 or IgG4. In a particular embodiment, the antibody is an IgG1 antibody. In some embodiments, the antibody is a human, humanized, or chimeric antibody.

Also provided is an isolated polynucleotide encoding the described anti-αvβ8 integrin antibody or an antigen binding fragment thereof; a prokaryotic, eukaryotic, or mammalian vector or vectors; and host cells, (prokaryotic, eukaryotic, or mammalian), suitable for encoding and expressing the anti-αvβ8 integrin antibody or an antigen binding fragment thereof as described.

In other aspects, antibodies useful in the described methods and compositions include immunoglobulins, monoclonal antibodies (including full-length monoclonal antibodies), polyclonal antibodies, multispecific antibodies formed from at least two different αvβ8 integrin epitope binding fragments (e.g., bispecific antibodies), human antibodies, humanized antibodies, camelid antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g., the antigen binding portion), disulfide-linked Fvs (dsFv), intrabodies, and antigen or epitope-binding fragments of any of the above. In particular, suitable antibodies include immunoglobulin molecules and immunologically and functionally active fragments of immunoglobulin molecules, e.g., molecules that contain at least one antigen-binding site.

Anti-αvβ8 integrin antibodies encompass monoclonal human, humanized or chimeric anti-αvβ8 integrin antibodies. Anti-αvβ8 integrin antibodies used in compositions and methods described herein can be naked antibodies, immunoconjugates, or fusion proteins. In certain embodiments, an anti-αvβ8 integrin antibody is a human, humanized, or chimeric antibody of the IgG isotype, particularly an IgG1, IgG2, IgG3, or IgG4 human isotype, or any IgG1, IgG2, IgG3, or IgG4 allele found in the human population. Antibodies of the human IgG class have advantageous functional characteristics, such as a long half-life in serum and the ability to mediate various effector functions (Monoclonal Antibodies: Principles and Applications, Wiley-Liss, Inc., Chapter 1 (1995)). The human IgG class antibody is further classified into the following subclasses: IgG1, IgG2, IgG3 and IgG4. In an embodiment, the anti-αvβ8 integrin antibody is of the human IgG1 subclass or isotype. The human IgG1 subclass has high ADCC activity and CDC activity in humans (Clark, Chemical Immunology, 65, 88 (1997)). In an embodiment, the anti-αvβ8 integrin antibody is a humanized antibody containing human framework regions and CDRs from a parent antibody, such as the MEDI-hu37E1B5 antibody. In another embodiment, the anti-αvβ8 integrin antibody comprises an optimized amino acid sequence to improve one or more antibody properties, including specificity for antigen, function, stability, half-life/longevity and the like.

### Treatment Methods Involving Administration of an Anti-αvβ8 Integrin Antibody

The described methods provide treatment of kidney disease, especially fibrotic kidney disease, and, in particular, chronic kidney disease (CKD) in which kidney function is reduced over a period of time and extensive fibrosis of kidney tissue typically occurs and is exacerbated over time. In general, the five stages of CKD are: Stage 1, characterized by kidney damage with normal kidney function (estimated glomerular filtration rate (GFR)≥90 mL/min per 1.73 m²) and persistent (≥3 months) proteinuria; Stage 2, characterized by kidney damage with mild loss of kidney function (estimated GFR 60-89 mL/min per 1.73 m²) with or without persistent (≥3 months) proteinuria; Stage 3, characterized by mild-to-severe loss of kidney function (estimated GFR 30-59 mL/min per 1.73 m²); Stage 4, characterized by severe loss of kidney function (estimated GFR 15-29 mL/min per 1.73 m²); and Stage 5, characterized by kidney failure requiring dialysis or transplant for survival. Stage 5 CKD is also known as ESRD (estimated GFR<15 mL/min per 1.73 m²). Glomerular filtration rate (GFR), measured in milliliters per minute (mL/min), refers to the rate at which the kidneys filter wastes and extra fluids from the blood.

The described methods involving administration of an anti-αvβ8 integrin antibody or an antigen-binding fragment thereof are also useful for treating kidney disease and/or fibrosis associated with damage or injury to kidney cells and tissue, as caused, for example, by diabetic nephropathy (DN), chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant, and the like. General and localized tissue inflammation in the kidney contributes to the pathophysiology and progression of diabetic nephropathy. The conditions of hyperglycemia and hypertension that typically accompany diabetic nephropathy, can further lead to glomerular hypertension and mechanical stress on kidney cells and tissue, podocyte injury and detachment, inflammation of the glomerulus and inflammation of the kidney tubules, all of which results in fibrosis (scarring) in the kidney, and more particularly, in the kidney glomerulus and tubules.

### Combination Treatments

In another embodiment, one or more of the anti-αvβ8 integrin antibodies may be administered in conjunction with another drug, medication, or therapeutic agent or compound, such as would be provided to a patient having kidney disease or CKD. As is frequently the case, individuals who have kidney disease or CKD also have high blood pressure. Medicines and drugs that lower blood pressure help to maintain blood pressure in a target range and delay or stop further kidney damage. Common blood pressure medications include, without limitation, acetylcholine esterase (ACE) inhibitors, angiotensin II receptor blockers (ARBs), beta blockers, calcium channel blockers, direct renin inhibitors, diuretics and vasodilators. Medications and drugs that are administered to treat the symptoms and complications of CKD include, without limitation, erythropoietin (EPO), (recombinant human erythropoietin, rhEPO), electrolyte imbalance correcting medicines, diuretics, ACE inhibitors and ARBs, as well as iron therapy and vitamin D.

In co-therapy, one or more anti-αvβ8 integrin antibodies may be optionally included in the same pharmaceutical composition as the other drug or medication. Alternatively, an anti-αvβ8 integrin antibody may be in a separate pharmaceutical composition and may be administered at the same time or at a different time from one or more other drugs or medications. An anti-αvβ8 integrin antibody as described herein, or a pharmaceutical composition comprising the anti-αvβ8 integrin antibody is suitable for administration prior to, simultaneously with, or following the administration of another drug or medication, or a pharmaceutical composition comprising the drug or medication. In certain instances, the administration of one or more of the anti-αvβ8 integrin antibodies to a subject overlaps with the time of administration of another or companion drug or medication provided separately or in a separate composition.

### Pharmaceutical Compositions and Formulations

The present disclosure encompasses the use of pharmaceutical compositions and formulations comprising one or more of the described anti-αvβ8 integrin antibodies and one or more pharmaceutically acceptable excipients, carriers and/or diluents. In certain embodiments, the compositions may comprise one or more other biologically active agents (e.g., inhibitors of proteases).

Non-limiting examples of excipients, carriers and diluents include vehicles, liquids, buffers, isotonicity agents, additives, stabilizers, preservatives, solubilizers, surfactants, emulsifiers, wetting agents, adjuvants, etc. The compositions can contain liquids (e.g., water, ethanol); diluents of various buffer content (e.g., Tris-HCl, phosphate, acetate buffers, citrate buffers), pH and ionic strength; detergents and solubilizing agents (e.g., Polysorbate 20, Polysorbate 80); anti-oxidants (e.g., methionine, ascorbic acid, sodium metabisulfite); preservatives (e.g., Thimerosol, benzyl alcohol, m-cresol); and bulking substances (e.g., lactose, mannitol, sucrose). The use of excipients, diluents and carriers in the formulation of pharmaceutical compositions is known in the art, see, e.g., Remington's Pharmaceutical Sciences, 18th Edition, pages 1435-1712, Mack Publishing Co. (Easton, Pa. (1990)), which is incorporated herein by reference in its entirety.

By way of nonlimiting example, carriers can include diluents, vehicles and adjuvants, as well as implant carriers, and inert, non-toxic solid or liquid fillers and encapsulating materials that do not react with the active ingredient(s). Non-limiting examples of carriers include phosphate buffered saline, physiological saline, water, and emulsions (e.g., oil/water emulsions). A carrier can be a solvent or dispersing medium containing, e.g., ethanol, a polyol (e.g., glycerol, propylene glycol, liquid polyethylene glycol, and the like), a vegetable oil, and mixtures thereof.

Formulations comprising one or more of the anti-αvβ8 integrin antibodies for parenteral administration can be prepared, for example, as liquid solutions or suspensions, as solid forms suitable for solubilization or suspension in a liquid medium prior to injection, or as emulsions. Sterile injectable solutions and suspensions can be formulated according to techniques known in the art using suitable diluents, carriers, solvents (e.g., buffered aqueous solution, Ringer's solution, isotonic sodium chloride solution), dispersing agents, wetting agents, emulsifying agents, suspending agents, and the like. Sterile fixed oils, fatty esters, polyols and/or other inactive ingredients can also be used. In addition, formulations for parenteral administration can include aqueous sterile injectable solutions, which can contain antioxidants, buffers, bacteriostats, and solutes that render the formulation isotonic with the blood of the intended subject and aqueous and nonaqueous sterile suspensions, which can contain suspending agents and thickening agents. Injection solutions and suspensions can be prepared from sterile powders, granules, and tablets.

Embodiments include sterile pharmaceutical formulations of anti-αvβ8 integrin antibodies that are useful as treatments for kidney diseases. Such formulations would inhibit the binding of ligands to the αvβ8 integrin, thereby effectively treating pathological conditions where, for example, tissue αvβ8 integrin is abnormally elevated. Anti-αvβ8 integrin antibodies may possess adequate affinity to potently inhibit αvβ8 integrin activity, and may have an adequate duration of action to allow for infrequent dosing in humans. A prolonged duration of action will allow for less frequent and more convenient dosing schedules by alternate parenteral routes such as subcutaneous or intramuscular injection.

Sterile formulations can be created, for example, by filtration through sterile filtration membranes, prior to or following lyophilization and reconstitution of the antibody. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

For therapeutic use, e.g., in the treatment of kidney disease, especially, kidney fibrosis, an anti-αvβ8 integrin antibody or an antigen binding fragment thereof, may be administered at a dose depending upon the requirements of the patient, the physical health and characteristics of the patient and the severity of the condition, e.g., the stage of CKD, being treated. For example, dosages can be empirically determined considering the type and stage of kidney disease and/or fibrosis diagnosed in a particular patient. The dose administered to a patient, in the context of the present compositions and methods should be sufficient to result in a beneficial therapeutic response in the patient over a given period of time. The size of the dose also will be determined by the existence, nature, and extent of any adverse side effects that accompany the administration of the antibody dose and/or the dose in combination with another therapeutic agent in a particular patient. The determination of the proper dose for a particular patient and situation is within the skill of a medical practitioner. In general, treatment is initiated using smaller doses, which are less than the optimum dose of the therapeutic. Thereafter, the dose is increased by small increments until effectiveness, such as optimum effectiveness, is achieved. For convenience and if desired, the total daily dosage may be divided and administered in portions during the day. Treatment with a determined or optimum dose may be continued for a short time period (e.g., hours or days), or over a longer time period (e.g., days, weeks, months, years).

### Detection Methods

In some embodiments, the anti-αvβ8 integrin antibody is used for detection, for example, for imaging or to determine the presence of αvβ8 integrin in vivo, ex vivo, or in vitro. In such embodiments, the antibody is labeled directly or indirectly with a detectable moiety. Accordingly, in some embodiments, methods are provided for determining the presence of αvβ8 integrin in a biological sample obtained from a subject (in vitro, ex vivo, or in vivo), which involves contacting the biological sample with a labeled anti-αvβ8 integrin antibody as described herein and detecting the presence of the labeled antibody bound to αvβ8 integrin, thereby determining the presence of αvβ8 integrin in the sample. Such methods may be used to diagnose kidney disease or a kidney-related condition such as kidney fibrosis, inflammation, or CKD.

In one embodiment, the antibody is conjugated to an “effector” moiety or molecule, which can be, without limitation, labeling moieties, such as radioactive labels or fluorescent labels, or a therapeutic moiety or molecule. In an embodiment, an effector moiety or molecule may include, but is not limited to, an anti-tumor drug, a toxin, a cytotoxic agent, a radioactive agent, a cytokine, a second antibody, or an enzyme. In another embodiment, the activity of the therapeutic moiety or molecule is modulated by virtue of its being conjugated to the antibody. In another embodiment, the antibody is linked to an enzyme that converts a prodrug into a cytotoxic agent.

An immunoconjugate comprising the antibody or an antigen binding fragment thereof can be used to target an effector moiety or molecule to a cell that expresses αvβ8 integrin on its surface, particularly diseased kidney cells and tissue, e.g., CKD kidney cells and tissue. Nonlimiting examples of cytotoxic agents that can be effector molecules include radioisotopes, ricin, doxorubicin, daunorubicin, taxol, ethiduim bromide, mitomycin, etoposide, tenoposide, vincristine, vinblastine, colchicine, dihydroxyanthracin dione, actinomycin D, diphteria toxin, Pseudomonas exotoxin (PE) A, PE40, abrin, steroids, glucocorticoids and other chemotherapeutic agents. Detectable markers include, without limitation, radioisotopes, fluorescent compounds, bioluminescent or chemiluminescent compounds, metal chelators, or enzymes.

In an embodiment, an anti-αvβ8 integrin antibody or an antigen binding fragment thereof is used as a therapeutic agent to reduce, abrogate, attenuate, decrease, block, or inhibit TGF-β activation in the kidneys, particularly, diseased or CKD kidneys, of an individual in need, either by itself (unconjugated), or conjugated to a detectable label or an effector moiety, such as an adjunct therapeutic treatment agent, such as a suitable treatment or therapeutic for kidney disease or CKD.

A “detectable label or moiety” may be a diagnostic agent or component that is detectable by a physical or chemical means, e.g., spectroscopic, radiological, photochemical, biochemical, immunochemical means, and the like. By way of example, detectable labels include radiolabels (e.g., ¹¹¹In, ⁹⁹mTc, ¹³¹I, ⁶⁷Ga) as well as other FDA-approved imaging agents. Additional labels may include ³²P, fluorescent dyes, electron-dense reagents, enzymes, biotin, digoxigenin, or haptens and proteins or other molecules that can be made detectable, for example, by incorporating a radiolabel into the targeting agent. Any method known in the art for conjugating a nucleic acid or a nanocarrier to the label can be used, such as by using methods as described in Hermanson, Bioconjugate Techniques 1996, Academic Press, Inc., San Diego.

A “labeled” or “tagged” antibody or agent is one that is bound, either covalently, through a linker or a chemical bond, or noncovalently, through ionic, van der Waals, electrostatic, or hydrogen bonding, to a label that allows the detection of the presence of the antibody, an antigen binding fragment thereof, or agent by detecting the label that is bound to the antibody or agent. Techniques for conjugating detectable and therapeutic agents to antibodies are known and practiced by those in the art, for example, as described in Arnon et al., “Monoclonal Antibodies For Immunotargeting Of Drugs In Cancer Therapy”, in Monoclonal Antibodies And Cancer Therapy, Reisfeld et al. (Eds.), pp. 243-56 (Alan R. Liss, Inc. 1985); Hellstrom et al., “Antibodies For Drug Delivery” in Controlled Drug Delivery (2nd Ed.), Robinson et al. (Eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (Eds.), pp. 475-506 (1985); and Thorpe et al., “The Preparation And Cytotoxic Properties Of Antibody-Toxin Conjugates”, Immunol. Rev., 62:119-58 (1982).

### Modes of Administration

In addition to the administration regimens described herein, an anti-αvβ8 integrin antibody or an antigen binding fragment thereof, or pharmaceutical compositions or formulations comprising an anti-αvβ8 integrin antibody or an antigen binding fragment thereof, can be administered to subjects by modes and routes that are suitable for administering and/or delivering a biologic drug, such as a protein or antibody, to a subject. In general, suitable biological delivery or administration methods embrace parenteral administration modes or routes. Such delivery methods include, without limitation, subcutaneous (SC) delivery, subcutaneous injection or infusion, intravenous (IV) delivery, e.g., intravenous infusion or injection or IV push. Other delivery and administration modes or regimens may include, without limitation, intra-articular, intra-arterial, intraperitoneal, intramuscular, intradermal, rectal, transdermal or intrathecal. In particular embodiments, the anti-αvβ8 integrin antibody is provided to a subject by intravenous administration, e.g., IV infusion or a bolus IV injection. In another particular embodiment, the anti-αvβ8 integrin antibody is provided to a subject by subcutaneous injection, such as a single subcutaneous injection.

An anti-αvβ8 integrin antibody can be administered in a chronic treatment regimen. The antibody can be administered for a period of time or a predetermined period of time followed by a period of no treatment. A dosing regimen or cycle can also be repeated. In some embodiments, the treatment (e.g., administration of the anti-αvβ8 integrin antibody) involves the administration of a first dose, followed by a second dose and/or one or more subsequent maintenance doses, e.g., for a time period comprising multiple days. Subsequent or maintenance doses may be administered at periodic intervals, e.g., weekly intervals, such as 1 week, 2 weeks, 3 weeks, or longer, e.g., 4 weeks, 5 weeks, 6 weeks, 7 weeks, 8 weeks, 9 weeks, 10 weeks, 11 weeks, 12 weeks, or at monthly intervals, or longer intervals, such as years, following the initial, second, or subsequent doses.

It is also contemplated that the anti-αvβ8 integrin antibody can be administered by direct delivery, e.g., infusion or injection, at or near a site of disease, as practicable. Injection in or near the kidney or kidney tissue may be useful. It is also contemplated that the anti-αvβ8 integrin antibody can be administered by implantation of a depot, which releases the antibody at the target site of action, such as in kidney tissue. Alternative modes of administration or delivery of the anti-αvβ8 integrin antibody may include inhalation (e.g., inhaler or aerosol spray), intranasal delivery, or transdermal delivery (e.g., by means of a patch on the skin). In addition, administration may be by osmotic pump (e.g., an Alzet pump) or mini-pump (e.g., an Alzet mini-osmotic pump), allowing for controlled, continuous and/or slow-release delivery of the anti-αvβ8 integrin antibody, or a pharmaceutical composition thereof, over a pre-determined period. The osmotic pump or mini-pump can also be implanted subcutaneously at or near the kidney or kidney tissue as the target site.

### Kits

Also provided are kits for the treatment of kidney disease, such as kidney disease involving fibrosis, e.g., CKD or DN. In an embodiment, the kit includes a composition, e.g., a therapeutic composition, containing an effective amount of an anti-αvβ8 integrin antibody, or an antigen binding fragment thereof. In an embodiment, the anti-αvβ8 integrin antibody, or an antigen binding fragment thereof, is in unit dosage form.

In some embodiments, the kit comprises a sterile container which comprises the anti-αvβ8 integrin antibody, or an antigen binding fragment thereof, e.g., in aqueous or lyophilized form. If the antibody is in a lyophilized form, the kit may include a container with an appropriate diluent, excipient, or vehicle for admixing with the dried antibody to prepare a solution containing the antibody, suitable for administration, e.g., intravenous administration. The containers can be ampoules, bottles, vials, tubes, bags, pouches, blister-packs, or other suitable container forms known in the art. Such containers can be made of plastic, glass, laminated paper, metal foil, or other materials suitable for holding medicaments, e.g., in aqueous or dried form. The containers can be in boxes for protection from damage or breakage. One or more syringes for antibody dilution and/or for administration may be included in the kit.

The kit may further provide instructions for administering the anti-αvβ8 integrin antibody, or a composition containing the antibody, to a subject having kidney disease, fibrotic kidney disease, e.g., CKD or DN. The instructions will generally include information about the use of the antibody or the composition for the treatment of kidney disease, fibrotic kidney disease, e.g., CKD or DN. In other embodiments, the instructions include one or more of the following: description of the therapeutic antibody; dosage schedule and administration for treatment of kidney disease, fibrotic kidney disease, e.g., CKD or DN, or symptoms thereof; dosage information; precautions; warnings; indications; counter-indications; overdosage information; adverse reactions; animal pharmacology; clinical studies; and/or references. The instructions may be printed directly on the container (when present), on a label applied to the container, or on a separate sheet, pamphlet, card, or folder supplied in the kit or with the container in the kit.

The present disclosure encompasses, unless otherwise indicated, conventional techniques of molecular biology (including any recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of a polynucleotide encoding an anti-αvβ8 integrin antibody, or an antigen binding portion or fragment thereof polynucleotides, and/or anti-αvβ8 integrin antibody, or an antigen binding portion or fragment thereof polypeptides as described herein, and, as such, may be considered in making and practicing the invention.

The following examples are set forth to provide those of ordinary skill in the art with a complete disclosure and description of how to make and use therapeutic methods of the invention, and are not intended to limit the scope of what the inventors regard as their invention.

### EXAMPLES

### Example 1

### Anti-αvβ8 Integrin Antibodies

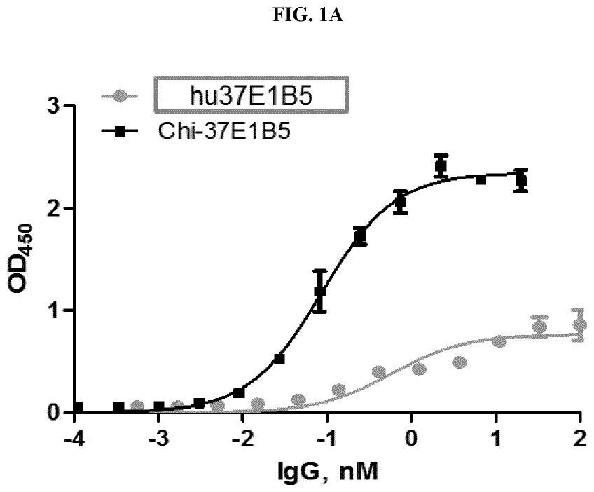

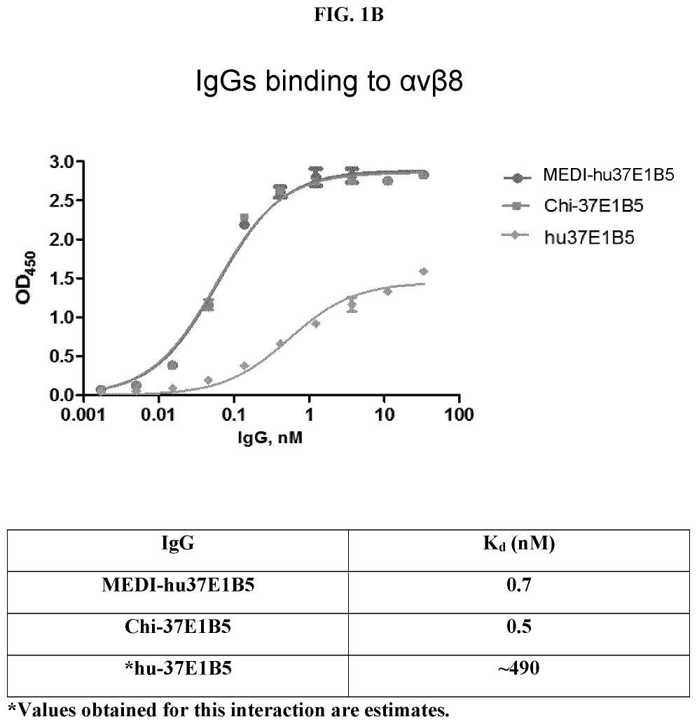

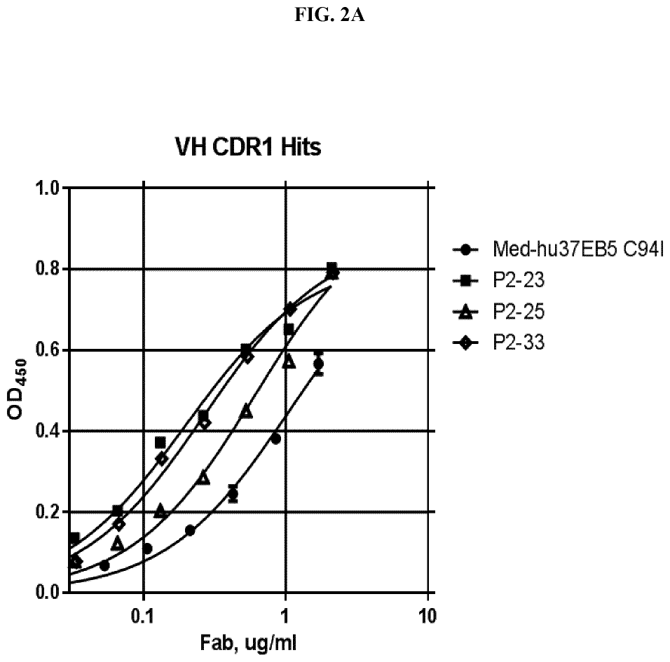

Several anti-αvβ8 integrin antibodies are embraced by the present disclosure and used in accordance with the methods, composition and products described herein, and/or as reference or control antibodies. Specifically, a chimeric anti-αvβ8 integrin antibody, called “Chi-37E1B5” herein, was in-licensed from The Regents of the University of California (UCSF). A second anti-αvβ8 integrin antibody, called “hu37E1B5” herein, was produced at MedImmune using a humanized sequence that was reported in published International PCT Application WO 2013/026004 (UCSF). When the hu37E1B5 antibody was evaluated in affinity binding studies, it was found to have very poor binding affinity for the αvβ8 integrin protein, as shown in FIG. 1A. Therefore, a third, humanized anti-αvβ8 integrin antibody, called “MEDI-hu37E1B5” herein, was generated using CDR grafting techniques known and practiced in the art. The CDRs used to produce the humanized MEDI-hu37E1B5 anti-αvβ8 integrin antibody were obtained from the above-described Chi-37E1B5 antibody. The MEDI-hu37E1B5 antibody exhibited a binding affinity for αvβ8 integrin protein that was similar to that of the Chi-37E1B5 antibody as shown in FIG. 1B. The amino acid sequences of the V_{H} and V_{L} regions and the CDRs of the MEDI-hu37E1B5 antibody are set forth in FIG. 6. Surprisingly, the binding affinity was retained upon humanization of the Chi-37E1B5 antibody. In contrast, the UCSF humanized antibody (“hu37E1B5”) showed very poor binding affinity upon humanization from Chi-37E1B5.

In addition, to obtain an anti-αvβ8 integrin antibody with improved binding affinity for αvβ8 integrin, a fourth, optimized, anti-αvβ8 integrin antibody, called “B5-15” herein, was generated from the MEDI-hu37E1B5 antibody as a parental antibody using affinity maturation techniques known and used in the art. The resulting B5-15 anti-αvβ8 integrin antibody (also called “optimized” or “affinity optimized” B5-15) exhibited an improved binding profile for αvβ8 integrin protein compared with that of the MEDI-hu37E1B5 anti-αvβ8 integrin antibody as shown in FIG. 4. The amino acid sequences of the V_{H} and V_{L} regions and the CDRs of the optimized B5-15 antibody are also set forth in FIG. 6.

### Humanization of the Chimeric Chi-37E1B5 Antibody by CDR Grafting

CDR grafting methods as known and practiced in the art were employed to humanize the mouse/human chimeric 37E1B5 (Chi-37E1B5) antibody and to produce the humanized MEDI-hu37E1B5 anti-αvβ8 integrin antibody. For humanization, the closest individual human germline framework (FW) with the same canonical class was selected to mimic the antibody folding structure. Critical murine FW residues for back mutations were identified, genes were synthesized, IgG was converted and produced by transient transfection using 293 cells, and the resulting antibodies were screened for binding to αvβ8 integrin. This process produced a fully humanized light chain clone, and a hybrid human germline FWs with 4 key mouse residues. The humanized MEDI-hu37E1B5 anti-αvβ8 integrin antibody resulting from the above methods was demonstrated to surprisingly retain the full binding activity of the original chimeric 37E1B5 (Chi-37E1B5) antibody (FIG. 1B). As observed in FIG. 1B, both the humanized MEDI-hu37E1B5 antibody and the Chi-37E1B5 antibody showed increased binding to αvβ8 integrin compared with the hu37E1B5 antibody, the sequence of which was reported in WO 2013/026004 as noted supra.

### Site-Saturation Mutagenesis and Affinity Maturation Leading to the Production of the Affinity Optimized, Humanized B5-15 Anti-αvβ8 Integrin Antibody

In addition, site-saturation mutagenesis was performed on the humanized MEDI-hu37E1B5 antibody to remove a Cys 94 residue (which has been shown to be a liability in antibody structure as it is associated with potential fragmentation/peptide cleavage of the antibody backbone) by first converting the residue to all of the other 19 amino acids. All of the resulting mutant antibodies were screened for binding to αvβ8 integrin via ELISA analysis. Depending on the residue at position 94, the binding affinity was reduced. The best αvβ8 integrin-binding mutants obtained from this procedure were called MEDI-hu37E1B5-C94I and MEDI-hu37E1B5-C94G. The MEDI-hu37E1B5-C94I mutant antibody had a roughly 3-fold reduction in αvβ8 binding affinity. The humanized MEDI-hu37E1B5-C94I antibody was selected for further analysis and affinity optimization.

Affinity maturation of the humanized MEDI-hu37E1B5-C94I, with the N-glycosylation site, was performed using parsimonious mutagenesis, an art-recognized method. Briefly, saturation point mutations to each CDR position of Medi-hu37E1B5-C94I were first generated. The mutations covered all 6 CDRs of the antibody V_{H} and V_{L} regions. A total of 6528 individual clones were screened (>4× redundancy) for binding to αvβ8 integrin. From these, 10 primary hits were identified: 3 were in V_{H}-CDR1; 2 were in V_{H}-CDR3; 1 was in V_{L}-CDR1; and 4 were in V_{L}-CDR3. All of the hits showed 2-5-fold improvement in binding to αvβ8 integrin.

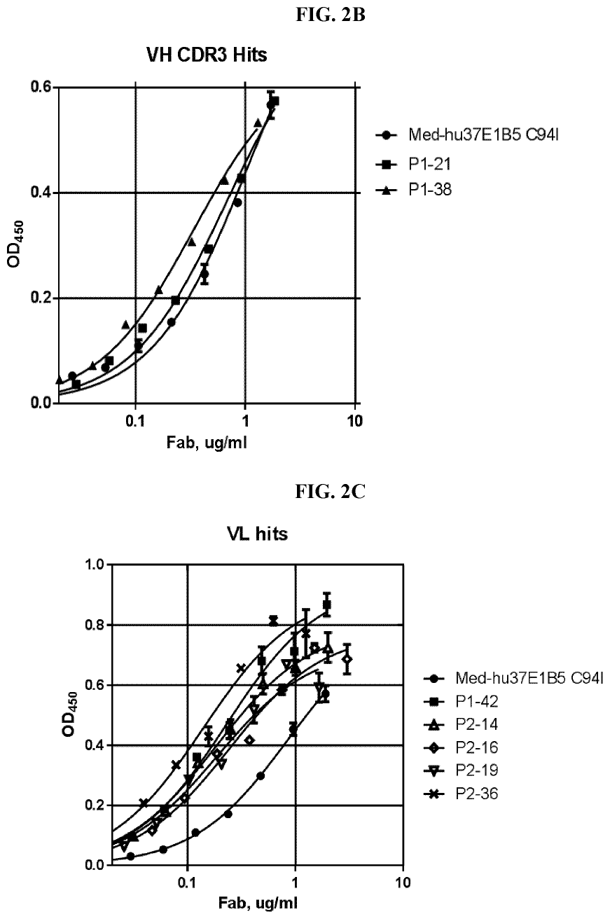

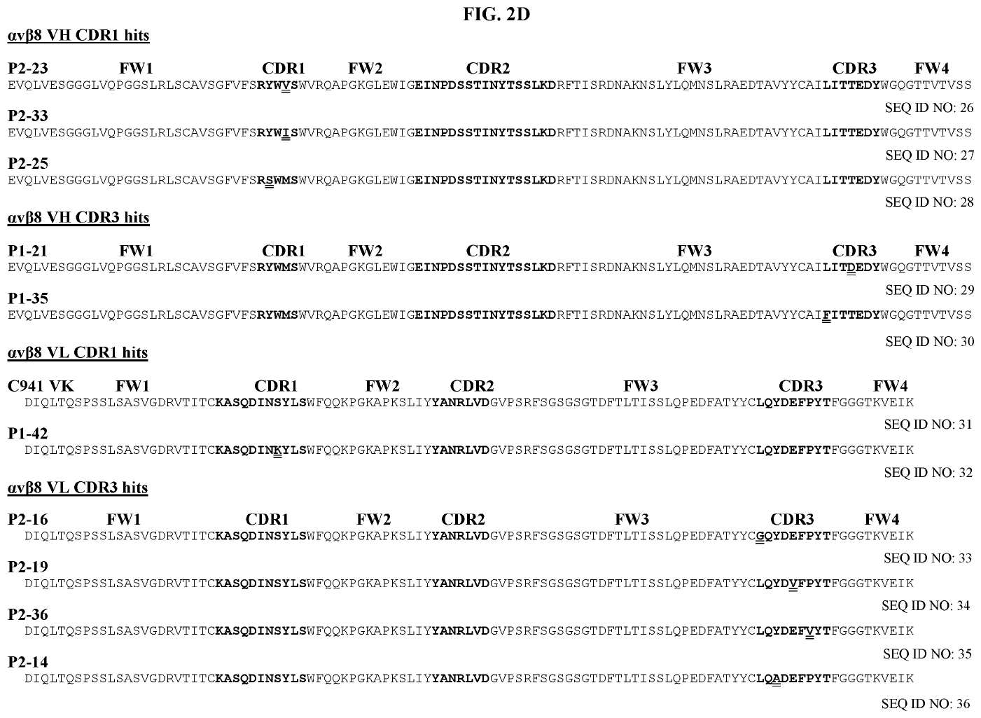

FIGS. 2A-2C present graphs showing the binding affinity analyses of the MEDI-hu37E1B5 C94I anti-αvβ8 integrin antibody and representative anti-αvβ8 integrin antibody “hits” (called “P1” or “P2” hits) identified in the screening analysis, e.g., V_{H}CDR1 hits (FIG. 2A), V_{H}CDR3 hits (FIG. 2B) and V_{L} hits (FIG. 2C). By way of example, for designating the antibody clone hits, “P” represents a given multi-well plate and the number following the P represents the well number in the plate.

FIG. 2D presents alignments of the amino acid sequences of the V_{H} and V_{L} regions of representative primary clonal anti-αvβ8 integrin antibody hits, designated “P2-23,” “P2-33,” “P2-25,” “P1-21,” “P1-35,” “P1-42,” “P2-16,” “P2-19,” “P2-36,” and “P2-14,” obtained from the screening of affinity matured anti-αvβ8 integrin antibody clones. The framework (FW1-FW4) regions and CDRs (CDR1-CDR3) in the V_{H} and V_{L} regions of the clones are designated above the sequences. Differences in the amino acid residues in the CDR regions are indicated by double underlining.

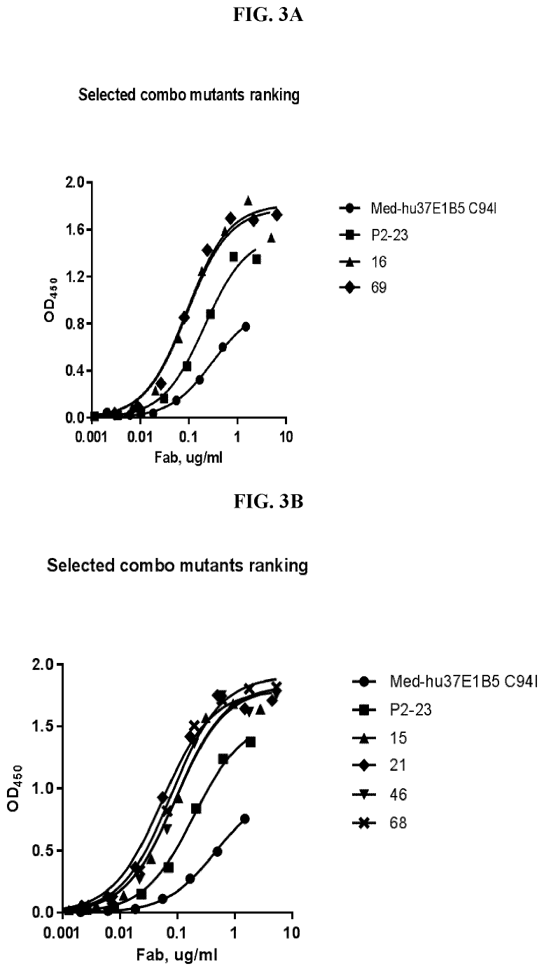

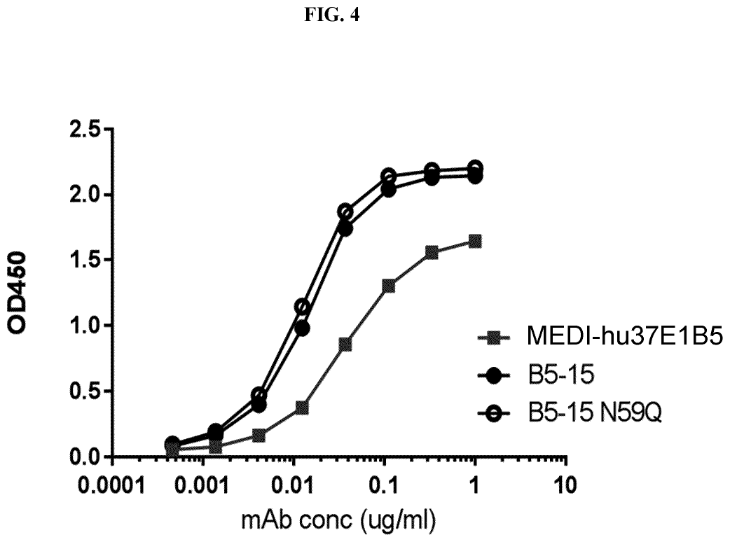

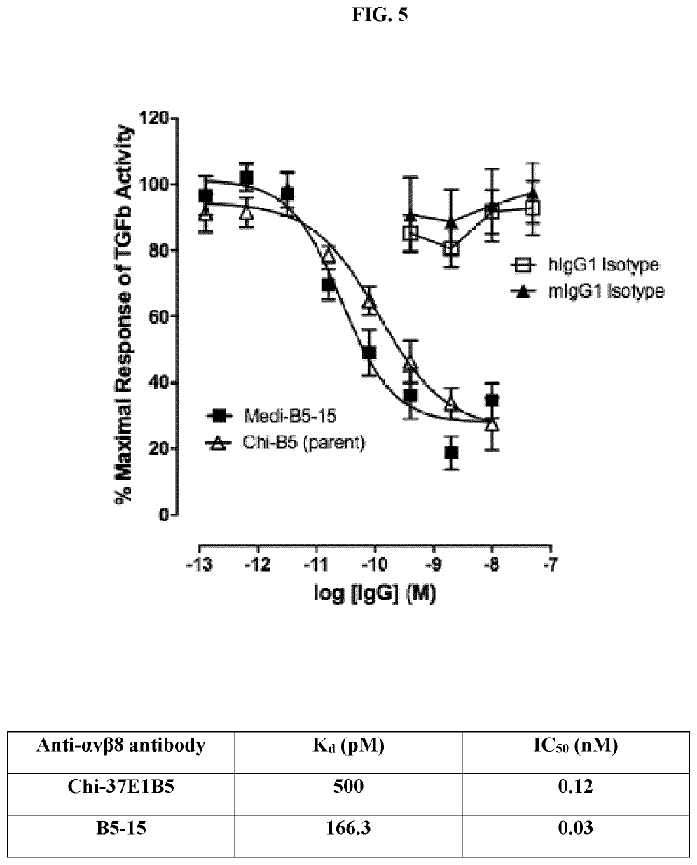

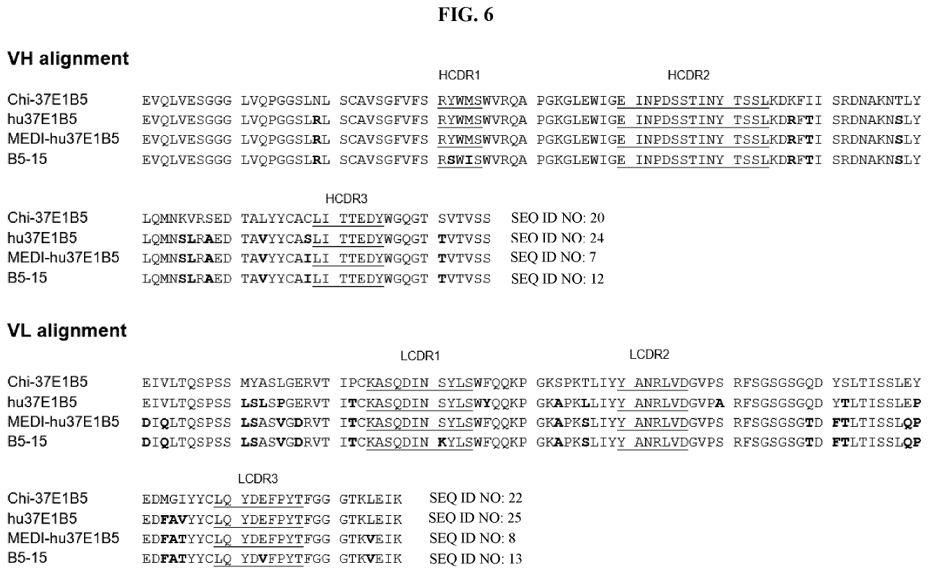

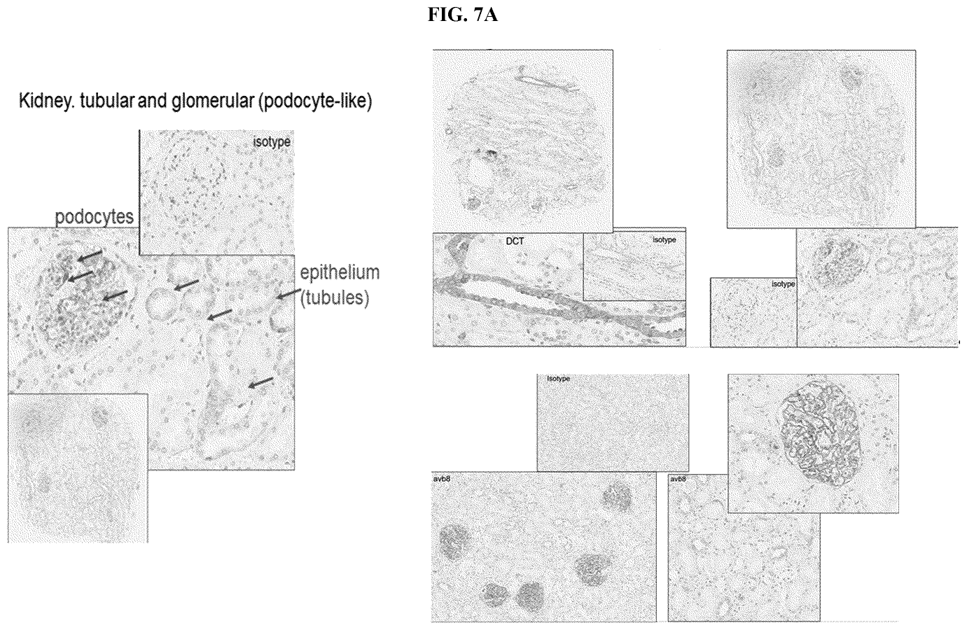

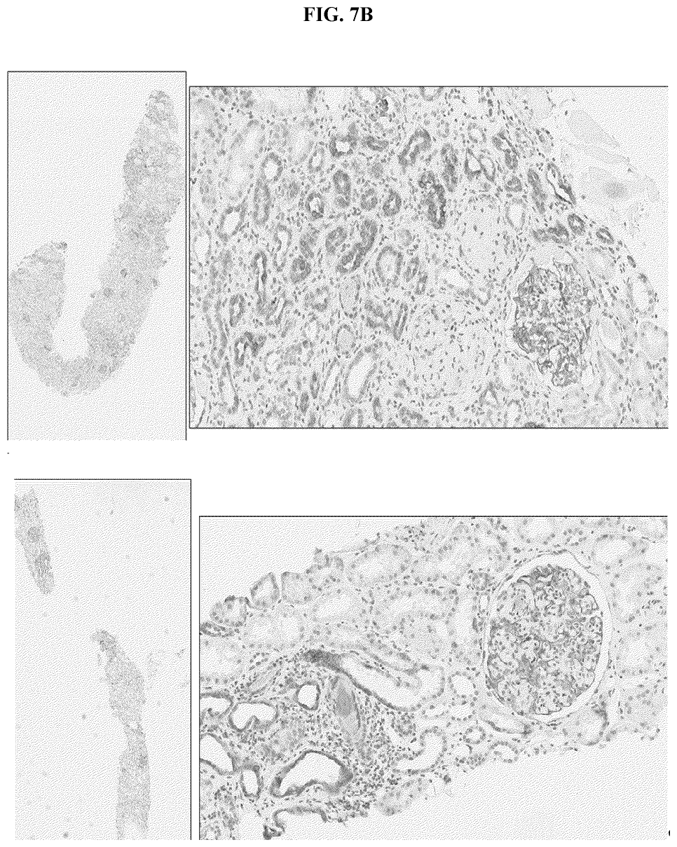

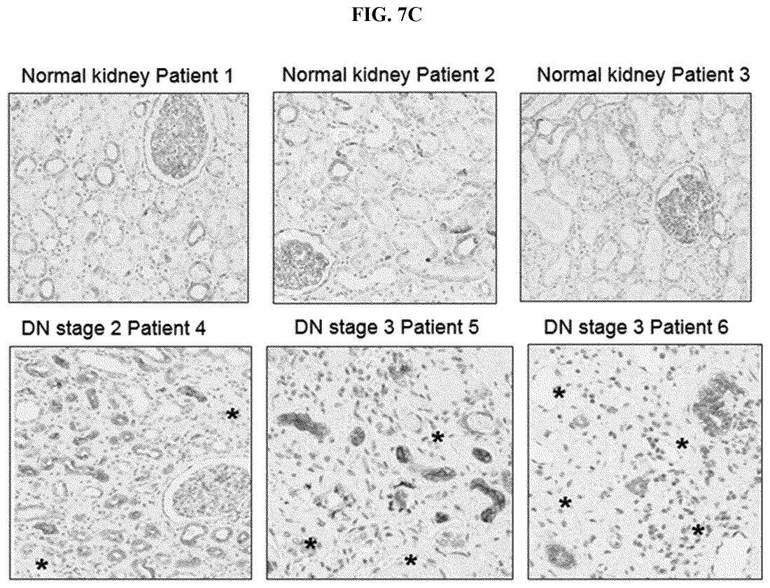

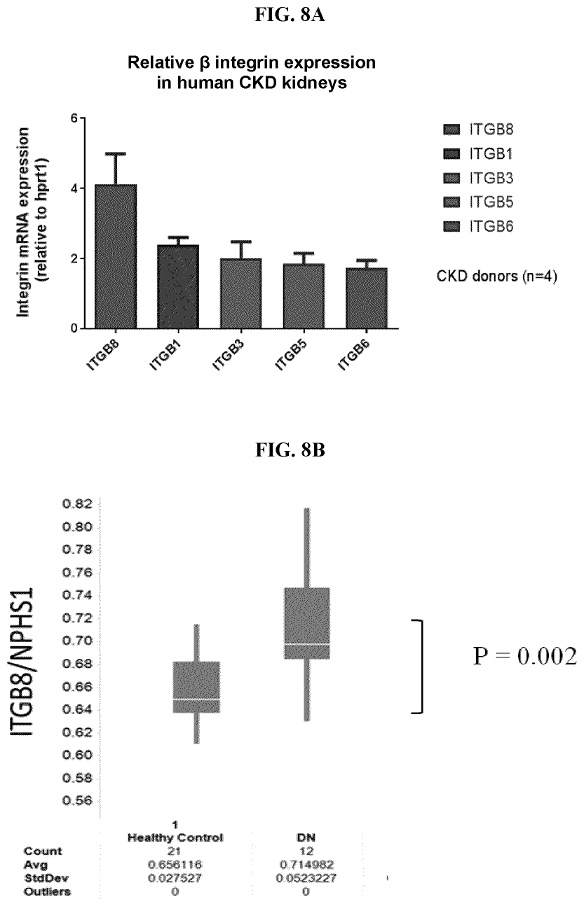

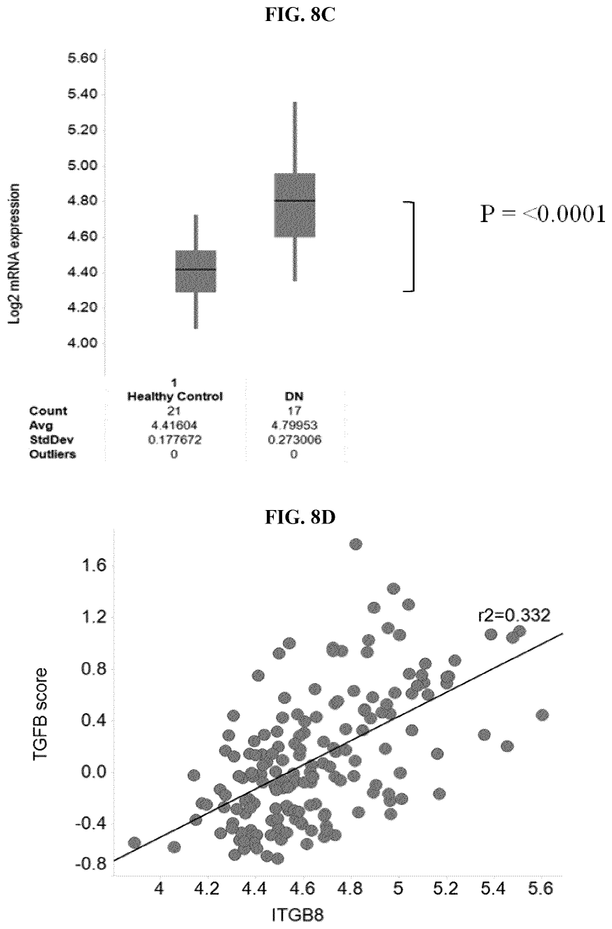

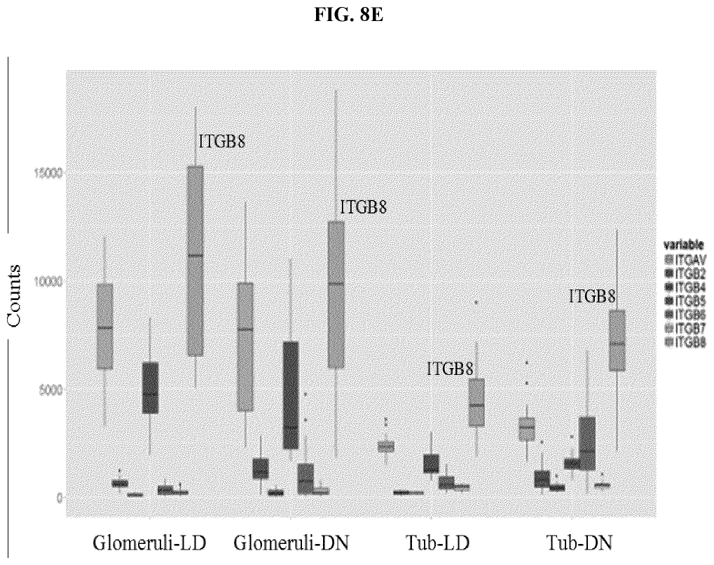

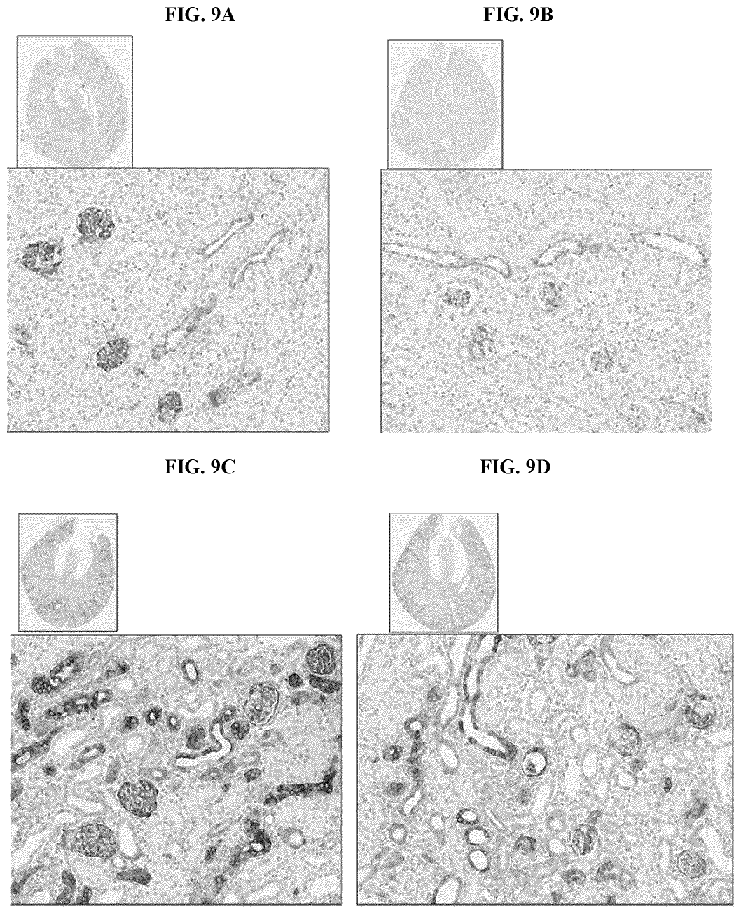

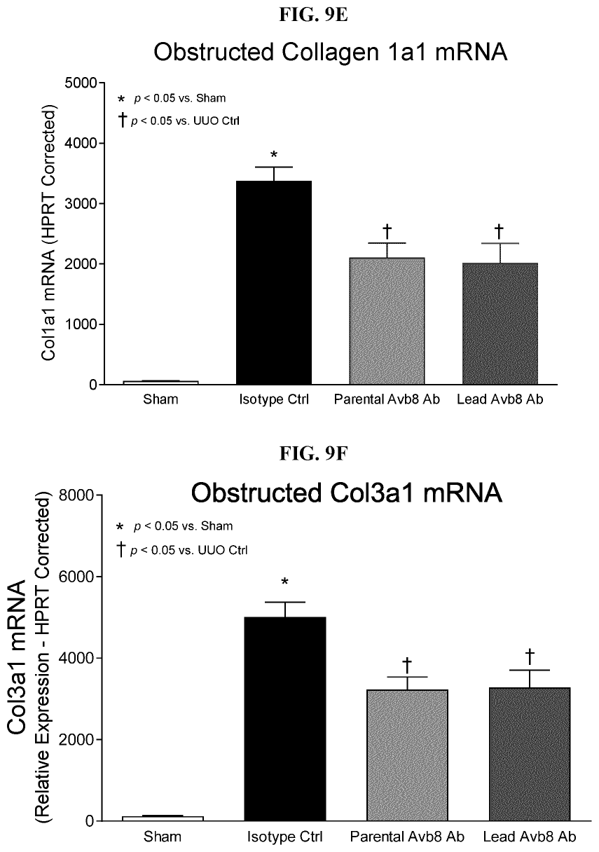

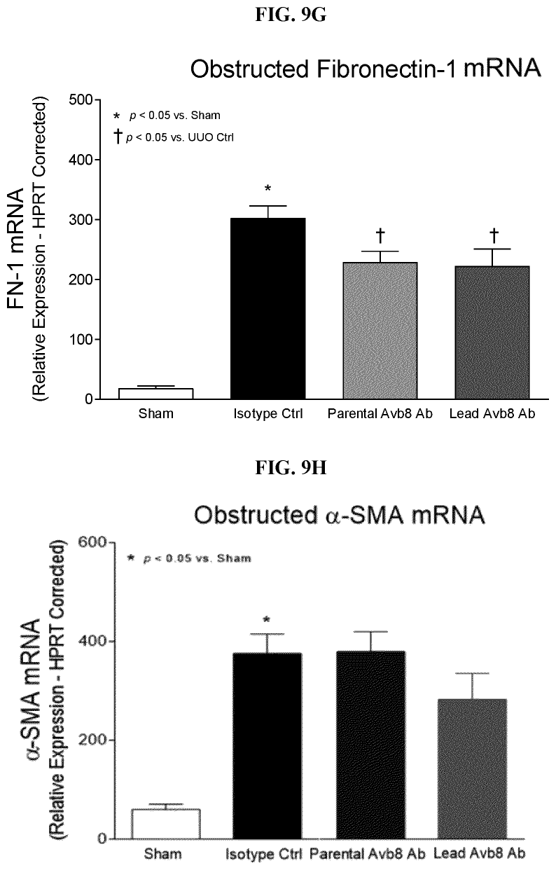

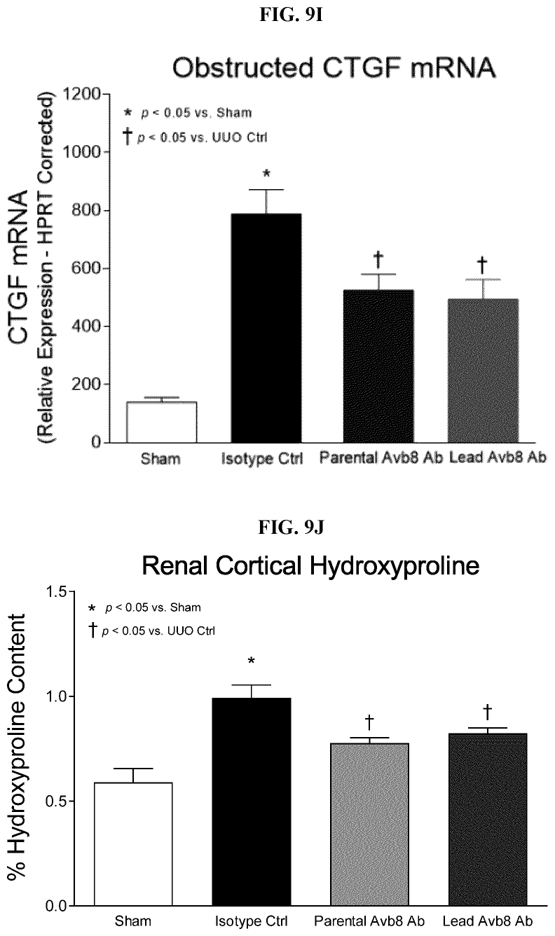

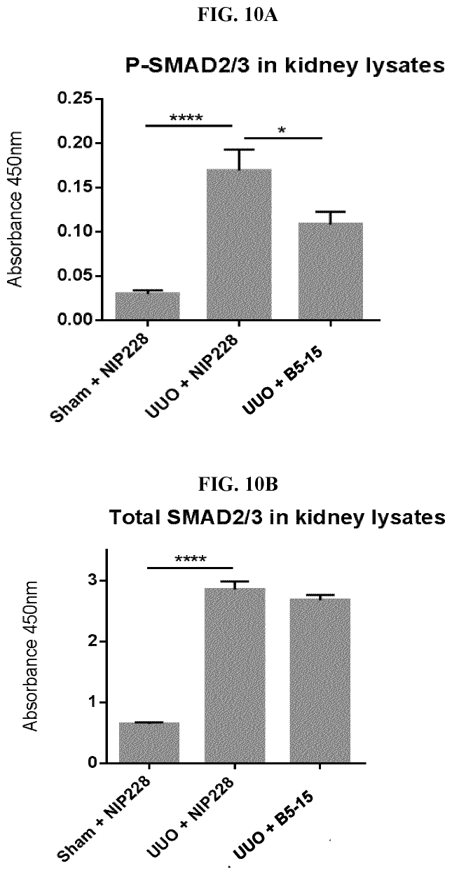

A combination library of the 10 most beneficial point mutations was then created in a combinatorial fashion. 4608 clones were screened for binding to αvβ8 integrin. 88 clones were selected for confirmation. 6 hits were identified from the combinatorial evaluation as showing additive improvement in binding to αvβ8 integrin compared with the best primary hit, P2-23. αvβ8 integrin binding data from the combination library screening are shown in FIG. 3A and FIG. 3B. The humanized and affinity optimized antibody, called B5-15 (“optimized B5-15” or “affinity optimized B5-15”) expressed in CHO (G22) cells was selected as the final, optimal antibody based on its higher binding affinity to αvβ8 integrin than MEDI-hu37E1B5 (FIG. 4) and on its higher in vitro potency in a TMLC luciferase assay than Chi-37E1B5 (FIG. 5).

### TGF-β Activation Bioassay

The TMLC luciferase bioassay is used in the art to measure TGF-β activation via integrins, such as αvβ8 integrin. The bioassay is based on a mink lung cell line, TMLC, that is stably transfected with a plasminogen activator inhibitor-1 (PAI-1) promoter fused to luciferase, as described, for example, in M. Abe et al., 1994, Anal. Biochem., 216(2):276-284; L. A. Randall et al., 1993, J. Immunol. Methods, 164(1):61-67; M. A. van Waarde et al., 1997, Anal. Biochem., 247(1):45-51); and I. Tesseur et al., 2006, BMC Cell Biology, 7:15 (https://doi.org/10.1186/1471-2121-7-15).

TGF-β activation was measured using transformed mink lung epithelial cells (TMLC) stably transfected with a portion of the plasminogen activated inhibitor 1 (PAI-1) promoter linked to a luciferase reporter (cells provided by Daniel Rifkin, New York University) and cultured as described previously (M. Abe et al., 1994, Anal. Biochem., 216(2):276-284). HeLa-B8 cells (1.5×104 cells/well) were co-cultured with TMLCs (1.5×104 cells/well) in a 96-well plate overnight in DMEM high glucose (Life Technologies/Thermo Fisher) supplemented with 10% FBS and 10 U/ml Penicillin G, 10 μg/mL streptomycin G sulfate with or without test antibody. After 16 hours, supernatants were removed and cells were lysed in 100 μL of cell lysis buffer (Promega) and luciferase activity determined using the luciferase assay system (Promega) by transferring 80 μL of lysate and mixing with 80 μL of substrate in a white walled clear bottom 96-well plate. Samples were read immediately on a luminometer and shown as either relative luciferase units (RLU) or percent maximal response, determined by using TMLCs alone as the baseline or 0% control and TMLCs co-cultured with HeLa-B8 cells as maximal or 100% response in the assay.

### Generation of the HeLa-B8 Cell Line

HeLa-B8 cells is a derivative of the HeLa cell line (ECACC). Briefly, confluent HeLa cells maintained in MEM (Life Technologies/Thermo Fisher) supplemented with 10% FBS, 1% non-essential amino acids (Life Technologies/Thermo Fisher) and 10 U/ml Penicillin G (Life Technologies/Thermo Fisher), 10 μg/mL streptomycin G sulfate and prior to use, cells were removed using accutase and resuspended in PBS at 1×106 cells/mL. LIVE/DEAD fixable aqua dead cell stain (Life Technologies/Thermo Fisher, 1:1000) was added to the cells on ice for 20 minutes. Cells were pelleted and washed in cold flow cytometry staining buffer (eBioscience). Recombinant 37E1B5-mIgG1 or isotype-mIgG1 (100 μg/ml of 1×106 cells/ml) was added to the cells and incubated on ice for 30 minutes. Cells were pelleted and washed and a secondary anti-mouse-Alexa-647 (Jackson ImmunoResearch, 1:200) added to the cells and incubated on ice for 30 minutes. Cells were pelleted and washed and resuspended at 10×106 cells/ml in HeLa cell medium containing 1% FBS. Cells were sorted on a BD FACSAria III cell sorter (BD Biosciences) using Chi-37E1B5 antibody. High αvβ8+ sorted cells were then cultured in complete HeLa cell medium, expanded and banked for future use. Cells remained positive for high αvβ8 expression for at least 1 month of culture.

### Characteristics of Humanized, Affinity Optimized B5-15 Anti-αvβ8 Integrin Antibody

The light chain (L) variable region (V_{L}, κ) amino acid (aa) sequence of the humanized and optimized B5-15 antibody polypeptide has 107 amino acid residues as follows:

B5-15 V_{L} (kappa (κ))
(SEQ ID NO: 13)
DIQLTQSPSSLSASVGDRVTITCKASQDINKYLSWFQQKPGKAPKSLIYYA
NRLVDGVPSRFSGSGSGTDFTLTISSLQPEDFATYYCLQYDVFPYTFGGGT
KVEIK (107 aa)

The heavy chain (H) variable region (V_{H}) amino acid sequence of the B5-15 antibody polypeptide has 116 amino acid residues as follows:

B5-15 V_{H}
(SEQ ID NO: 12)
EVQLVESGGGLVQPGGSLRLSCAVSGFVFSRSWISWVRQAPGKGLEWIGEI
NPDSSTINYTSSLKDRFTISRDNAKNSLYLQMNSLRAEDTAVYYCAILITT
EDYWGQGTTVTVSS (116 aa)

In particular, the light chain (L) variable region (V_{L}) of the B5-15 antibody includes three CDRs having the amino acid sequences as follows:

V_{L} CDR1:
(SEQ ID NO: 10)
KASQDINKYLS
V_{L} CDR2:
(SEQ ID NO: 5)
YANRLVD
V_{L} CDR3:
(SEQ ID NO: 11)
LQYDVFPYT

The heavy chain (H) variable region (V_{H}) of the B5-15 antibody includes three CDRs having the amino acid sequences as follows:

V_{H} CDR1:
(SEQ ID NO: 9)
RSWIS
V_{H} CDR2:
(SEQ ID NO: 2)
EINPDSSTINYTSSL
V_{H} CDR3:
(SEQ ID NO: 3)
LITTEDY

A comparison of the amino acid sequences of the V_{H} and V_{L} regions of Chi-37E1B5, hu37E1B5, MEDI-hu37E1B5 and B5-15 anti-αvβ8 integrin antibodies is presented in FIG. 6.

### Example 2

Immunohistochemistry (IHC) Detection Method for αvβ8 Integrin Expression in Formalin-Fixed Paraffin-Embedded (FFPE) Human Tissue Using an Anti-αvβ8 Integrin Antibody

### IHC Method

To prepare formalin-fixed paraffin-embedded (FFPE) tissue sections, slides onto which human tissue samples were affixed were removed from storage. The slides were appropriately labeled and loaded into Autostainer XL rack(s). The stained slides were dewaxed and rehydrated in tap water.

The slide racks were transferred to a pressure cooker containing Dako Antigen Retrieval Solution (Dako S1699). Heat Mediated Antigen Retrieval was performed for 2 minutes at pressure (SP DDCP_5024) with the following alterations: following antigen retrieval, the pressure cooker was allowed to cool and de-pressurize; the pressure cooker was placed in running tap water and the lid was removed; the pressure cooker was cooled for 5 minutes and its contents were then flushed with running tap water; the slides were removed and rinsed in running tap water for 5 minutes. Slides were blocked with peroxidase (3% Hydrogen Peroxide in methanol) for 10 minutes.

Immunohistochemistry was performed as follows:
PAP pen slides at appropriate locations (for drawing hydrophobic barriers on tissue);
Load slides onto Dako Autostainer;
Rinse in standard Dulbecco's PBS with 0.1% Tween 20 (PBST)×1 (one time);
Incubate slides in 2.5% Horse Serum (from ImmPRESS kit) 20 minutes;
Blow step; Incubate in either Calico antibodies CAL16 (a purified rabbit recombinant anti-αvβ8 integrin antibody) @ 1.0 ug/ml diluted in PBST, Dako rabbit immunoglobulin isotype control @ 1.0 ug/ml or Vector Ki67 at a 1:200 dilution as experiment control for 60 minutes;
Rinse in PBST×1;
Incubate in labeled polymer, Vector ImmPRESS™ HRP, horse anti-Rabbit IgG (Peroxidase) Polymer Detection Kit, (Catalog No. MP-7401) for 30 minutes;
Rinse in PBST×1;
Incubate in PBST for 5 minutes;
Rinse in PBST×1; Switch to hazardous waste ×1;
Incubate in DAB+ substrate/chromagen (Dako, K3468) for 5 minutes;
Rinse in Pure water (automatically done by Autostainer) ×1;
Unload slides from Dako Autostainer;
Counterstain with Gill I Haematoxylin, dehydrate and coverslip slides using program 9; and
Unload slides from Leica CV5030 Coverslipper and allow to dry/set.

TABLE 1

| | | COUNTERSTAIN AND DEHYDRATION | | |
|---|---|---|---|---|
| PROGRAM NO: 9 | | | TIME | |
| STEP | STATION | REAGENT | [M:S] | EXACT |
| 1 | WASH 5 | RUNNING TAP WATER | 3:00 | NO |
| 2 | 11 | HAEMATOXYLIN GILL I | 0:25 | YES |
| 3 | WASH 4 | RUNNING TAP WATER | 4:00 | NO |
| 4 | 12 | 1% ACID ALCOHOL | 0:02 | YES |
| 5 | WASH 2 | RUNNING TAP WATER | 5:00 | NO |
| 6 | 13 | 95% IMS | 1:30 | NO |
| 7 | 14 | IMS | 1:00 | NO |
| 8 | 15 | IMS | 1:30 | NO |
| 9 | 16 | IMS | 1:00 | NO |
| 10 | 17 | XYLENE | 2:00 | NO |
| 11 | 18 | XYLENE | 2:00 | NO |
| 12 | EXIT | XYLENE | /:/ | / |

Alternatively, one can perform the steps in “Program No: 9” manually. To do this, one can place the slides in the stated reagents for the time stated. One could use either the automated program or perform the steps manually.

### Example 3

αvβ8 Integrin is Preferentially Expressed in Kidney

IHC staining analysis as described in Example 2 was carried out on numerous tissue samples to determine αvβ8 integrin expression and distribution in human tissues. Table 2 below presents the results of the IHC analysis.

TABLE 2

| | | Number of | |
|---|---|---|---|
| | | samples that | |
| | | express αvβ8 | |
| Tissue | N | integrin | Location |
| Heart | 3 | 0 | |
| Lung | 3 | 0 | |
| Kidney | 3 | 3 | Glomerular and tubular |
| Spleen | 3 | 0 | |
| Lymph node | 3 | 0 | |
| Thymus | 3 | 2 | Epithelial cells surrounding |
| | | | Hassall's corpuscles |
| Tonsil | 3 | 2 | Trabecula stratified squamous |
| | | | epithelium |
| Liver | 3 | 0 | |
| Gall bladder | 3 | 1 | Weak cytoplasmic staining |
| | | | columnar epithelium |
| Pancreas | 3 | 1 | Small area of stroma positive |
| | | | expression |
| Brain | 3 | 1 | Weak homogeneous parenchyma |
| cerebellum | | | |
| Brain | 3 | 2 | Weak homogeneous parenchyma |
| cerebellum | | | |
| Thyroid | 3 | 0 | |
| Adrenal | 3 | 2 | Cortex cells - rare interstitial αv8β |
| | | | integrin staining detected |
| | | | Capillaries |
| Parotid | 3 | 0 | |
| Skin | 3 | 0 | |
| Skeletal | 3 | 0 | |
| muscle | | | |
| Stomach | 3 | 0 | |
| Ileum | 3 | 2 | Adventitia nerve cells |
| Colon | 3 | 0 | |
| Ovary | 3 | 1 | ? nerve cells |
| Fallopian tube | 3 | 1 | Rare weak columnar epithelium |
| Uterus | 3 | 0 | |
| myometrium | | | |
| Endometrium | 3 | 1 | Endometrial gland epithelium |
| Endocervix | 3 | 1 | Weak stratified squamous |
| Exocervix | 3 | 2 | Epithelial membrane. Columnar/ |
| | | | stratified squamous |
| Breast | 3 | 0 | |
| Placenta | 3 | 3 | Weak scanty trophoblast, rare |
| | | | membrane |
| Prostate | 3 | 1 | Moderate glandular epithelium |
| Testis | 3 | 2 | Weak cytoplasmic primary |
| | | | spermatocytes |
| Seminal | 3 | 2 | Glandular/vesicle epithelium |
| vesicles | | | |
| Bladder | 3 | 0 | |
| Ureter | 3 | 0 | |

As observed in Table 2, kidney tissue expresses a high level of αvβ8 integrin. In addition, αvβ8 integrin was found to be highly enriched in human kidney tissue compared with 33 other human tissue types, namely, heart, lung, spleen, lymph node, thymus, tonsil, liver, gallbladder, pancreas, brain cerebellum and cerebrum, thyroid, adrenal, parotid, skin, skeletal muscle, stomach, ileum, colon, ovary, fallopian tube, uterus myometrium, endometrium, endocervix, exocervix, breast, placenta, prostate, testis, seminal vesicle, bladder and ureter). In FIG. 7A (left-hand side), strong staining of αvβ8 integrin was observed in human kidney tissue, and particularly in the podocytes and epithelial cells of the tubules in kidney tissue. By contrast, staining was found to be weak, inconsistent, or nonexistent in the other tissue types that were examined.

For the IHC staining analyses presented in FIG. 7A, CAL16 clone anti-αvβ8 integrin rabbit monoclonal antibody (a purified rabbit recombinant anti-αvβ8 integrin antibody from Calico Biolabs Inc. (Pleasanton, Calif.)) was used. This antibody, which is commercially available, was optimized and validated for binding to αvβ8 integrin expressed in both human and mouse tissues.

### Example 4

Avβ8 Expression is Increased in the Kidney Tissue of Patients with Chronic Kidney Disease (CKD)

The expression of αvβ8 integrin was evaluated in human kidney tissue samples taken from patients with diabetic nephropathy (DN) and from individuals with normal kidney tissue as “healthy” controls. DN kidney tissue samples were obtained from Addenbrooke's Biobank and MedImmune (Gaithersburg) Biobank. Normal kidney samples were obtained from MedImmune (Cambridge) tissue bank. More specifically, samples from 9 patients having diabetic nephropathy chronic kidney disease, DN-CKD, were obtained by needle biopsy. Samples from 4 healthy ‘normal’ individuals were used as controls. In the normal samples, some areas of mild chronic inflammation were evident, but did not impact the study design or results (FIG. 7A, right-hand side).

As described in Example 3, the antibody used in the IHC staining experiments was CAL16 clone anti-αvβ8 integrin rabbit monoclonal antibody (purified rabbit recombinant antibody) from Calico Biolabs Inc. (Pleasanton, Calif.). After staining, the slides were reviewed by an experienced senior pathologist.

The results from this IHC staining analysis were as follows: In the healthy individuals, the glomeruli showed positive staining with the anti-αvβ8 integrin antibody compared with isotype-matched control antibody staining; the anti-αvβ8 integrin antibody staining was generally light (¾ samples), although one sample (¼) showed strong staining in podocytes (podocyte pattern). In kidney tubules, light multifocal staining was observed in cortical tubules, membrane, basal to apical. (FIG. 7A, right-hand side). Staining in the tubules did not appear to be in collecting ducts. The overall staining pattern of healthy human kidneys was mostly in the glomeruli, similar to healthy transgenic mice, while staining of αvβ8 integrin by IHC was observed in tubular structures in both CKD patients and in the UUO transgenic mice.

In the patients having DN-CKD, the extent of αvβ8 staining in the glomeruli was variable and was in relation to the degree of glomerular damage. The loss of podocytes in the diseased tissue correlated with less staining. Because of podocyte loss in diseased kidney, the staining intensity was variable. Staining of tubules in DN-CKD kidney tissue varied from light staining to strong staining of cytoplasm and membrane, mostly in areas of inflammation/fibrosis. An overall increased expression of αvβ8 integrin was observed in DN-CKD kidneys as evidenced by the staining pattern of the anti-αvβ8 integrin antibody. The overexpression was essentially seen in kidney tubules. (FIG. 7B). Based on the anti-αvβ8 integrin antibody staining, the change in expression of αvβ8 integrin in the DN-CKD kidney tissue appeared to adequately approximate αvβ8 integrin expression in mouse model kidneys showing tubulo-interstitial inflammation and fibrosis.

FIG. 7C presents photomicrographs of kidney tissue cells obtained from human patients having kidney disease. The kidney tissue cells were stained with an anti-αvβ8 integrin antibody and analyzed by IHC. The IHC staining results demonstrated that the αvβ8 integrin protein is upregulated in kidney cells and tissue of human patients with diabetic nephropathy (DN) compared with normal kidney cells and tissue (FIG. 7C, top row). In particular, in the kidney tissue samples obtained from DN and CKD patients, overexpression of αvβ8 integrin was essentially found in tubules (FIG. 7C, bottom row). The glomeruli of kidneys in DN patients showed decreased αvβ8 integrin expression, likely as a consequence of podocyte loss due to kidney tissue fibrosis and damage. The unstained areas in the kidney tissue samples from patients having Stage 2 and Stage 3 DN are fibrotic matrix that replaced functional nephrons, as designated by an asterisk (*) in FIG. 7C. This result highlights the importance of targeting αvβ8 integrin to protect functional epithelium. For the IHC staining analyses presented in FIG. 7C, CAL16 clone anti-αvβ8 integrin rabbit monoclonal antibody (a purified rabbit recombinant anti-αvβ8 integrin antibody from Calico Biolabs Inc. (Pleasanton, Calif.)) was used. This antibody, which is commercially available, was optimized and validated for binding to αvβ8 integrin expressed in both human and mouse tissues.

### Example 5

Itgb8 Gene is Upregulated in Kidneys from Individuals with CKD and has Elevated Expression Compared with Other β Integrins

Transcriptomics analyses provided evidence that kidneys of human CKD patients had higher expression of ITGB8 (which encodes for (38 integrin) compared with the kidneys of healthy human subjects. In these analyses, the relative (3 integrin family mRNA expression was measured in human CKD kidney homogenates. In brief, 1 punch (2 mm puncher) of kidney biopsies was homogenized in RLT lysis buffer using a TissueLyserII. RNA from the lysates was isolated with RNAeasy Mini kit columns. RNA concentration was measured with a Nanodrop and concentrations were adjusted to perform qPCR analyses using the TaqMan RNA to Ct 1-step Kit and specific probes for all the integrins, with the hprt-1 included as a housekeeping gene. FIG. 8A presents a bar graph showing the relative expression levels of mRNA encoding different isoforms of β integrins in kidneys from human patients having CKD. As seen in FIG. 8A, β8 integrin mRNA expression predominated that of the other β integrins (i.e., β1, β3, β5 and β6) in the kidneys of CKD patients.

In other experiments, the transcriptomic profiles of 157 patients having different degrees of CKD were analyzed and compared with those of living donors (LD). Twelve (12) of the 157 patients had diabetic neuropathy (DN). Glomerular and tubulo-interstitital compartments were separated and whole genome gene expression analysis was performed as described by S. Martini et al. (2014, 1 Am. Soc. Nephrol., 25(11):2559-2572). In this analysis, the expression of itgb8 mRNA was first assessed in the renal glomerular compartment in relation to nephrin (encoded by NPHS1 gene). Nephrin is a podocyte protein necessary for the proper functioning of the renal filtration barrier, which consists of fenestrated endothelial cells, the glomerular basement membrane, and the podocytes of epithelial cells. Mutations in NPHS1 are associated with congenital nephrotic syndrome. NPHS1 expression is an indicator of podocyte number. In CKD, as podocyte numbers decrease, there is a reduction in NPHS1 expression. FIG. 8B shows that itgb8 mRNA expression positively correlated with the podocyte marker gene, NPHS1, supporting the expression of this gene in kidney podocytes. To better assess itgb8 expression in the glomerular cortex taking into account podocyte loss, itgb8 expression was normalized by NPHS1. Data were therefore normalized for nephrin (encoded by the NPHS1 gene) expression to understand expression changes within podocytes under conditions of podocyte loss as in chronic kidney disease. FIG. 8C presents a box plot graph showing itgb8 mRNA expression was higher in the tubule-interstitium (TI) of DN patient kidney samples relative to its expression in living donors (LD) as healthy controls. FIG. 8D presents a dot plot graph showing that itgb8 mRNA expression was strongly correlated with the TGF-β activation score across CKD in the TI of patients with CKD, supporting the role of αvβ8 integrin in TGF-β activation in CKD.

In a separate cohort, the tubulo-interstitium (Tub) and glomerulus (Glom) of kidney samples obtained from 20 human patients with DN compared with the TI and glomerulus of kidney samples obtained from 19 LD patients were profiled by whole genome transcriptional profiling using RNAseq. The results showed that itgb8 mRNA expression increased in the tubulo-interstitium of DN patients (designated as “Tub-DN” in the graph) versus that in living donors (LD), (FIG. 8E). The finding of high itgb8 mRNA levels in the tubulo-interstitium of patients with kidney disease, i.e., diabetic nephropathy, correlates with conditions of renal damage and fibrosis in these kidney disease patients.

The key findings of these analyses were as follows: itgb8 mRNA expression was elevated in the glomeruli from DN patient samples after normalization to nephrin (NPHS1), a podocyte marker gene. itgb8 mRNA expression was elevated in the tubule-interstitium (TI) of DN patients. In the TI, itgb8 mRNA expression was positively correlated with a putative TGF-β activation score, consistent with a proposed role of αvβ8 integrin in controlling TGF-β activation in fibrotic diseases. Similar results were found following the analysis of mRNA expression of WT1, another podocyte marker gene (data not shown). These findings support the discovery that in human CKD kidney, αvβ8 integrin expression correlates with fibrosis in CKD, which is associated with the activation of TGF-β, an important player in causing and exacerbating kidney fibrosis.

### Example 6

### In Vivo Efficacy of Anti-αvβ8 Integrin Antibodies

A mouse model of fibrosis induction was used to study the in vivo efficacy of the anti-αvβ8 integrin antibody in treating fibrosis in the kidney. This model involved performing a procedure called unilateral ureteral occlusion (UUO), (unilateral ligation of the ureter), on the animals. For the model, male, humanized αvβ8 transgenic (Tg) mice underwent a sham or a UUO procedure involving five (5) and eight (8) day duration of injury. The Tg mice were produced by crossing a mouse in which the αvβ8 gene was knocked out (αvβ8 KO mouse) with a human αvβ8 BAC transgenic mouse. The generation of Tg mice expressing human ITGB8 gene is described, for example, in S. Minagawa et al., 2014, Sci. Transl. Med., 6(241):241ra79 (doi: 10.1126/scitranslmed.3008074). The humanized αvβ8 transgenic mice expressed human αvβ8 integrin mainly in the kidney glomerulus, in a pattern similar to that observed in healthy humans. The induction of fibrosis following ureteral ligation (UUO) increased αvβ8 integrin expression in kidney tubules, similar to what is observed in human CKD.

The test agent used was B5-15, the IgG1 humanized and sequence optimized anti-αvβ8 integrin antibody as described supra. The control antibody was an isotype-matched IgG antibody.

Protocol for the UUO Tg Mouse Model Study:

Model: 91 male humanized αvβ8 transgenic (Tg) mice underwent sham or a unilateral ureteral occlusion (UUO) procedure; 5- or 8-day duration of injury. The animals in the groups were dosed with respective antibody treatment every other day (EOD) on Days −1, 1, 3, 5 and 7. The sham-treated animals were administered vehicle on Days 0, 2, 4 and 6.

Mice Age at Study Inception: 92-121 days old.

Test Agents/Compounds: anti-αvβ8 integrin antibody (Chi-37E1B5 monoclonal antibody), B5-15 sequence optimized anti-αvβ8 integrin antibody), IgG isotype control and/or vehicle (PBS) were administered at doses, frequencies, and to groups as displayed in Table 3 below.

TABLE 3

| | | Compound and | | Surgical | |
|---|---|---|---|---|---|
| | | Administration | | Preparation | |
| N/group | | Interval | Dose and | Interval | |
| (Animal #s) | Strain | (Day (D)) | Route | (Day (D)) | Condition |
| n = 6 | Tg | IgG Control | 10 mg/kg | Once at D 0 | Sham |
| (3428-3433) | | D −1, D 1, D 3, D 5, D 7 | i.p. | | Surgery |
| | | Vehicle | N/A | | |
| | | D 0, D 2, D 4, D 6 | i.p. | | |
| n = 10 | Tg | IgG Control | 10 mg/kg | Once at D 0 | Permanent |
| (3434-3443) | | D −1, D 1, D 3 | i.p. | | UUO |
| | | Vehicle | N/A | | |
| | | D 0, D 2, D 4, D 6 | i.p. | | |
| n = 10 | Tg | Chi-37E1B5 | 10 mg/kg | Once at D 0 | Permanent |
| (3454-3463) | | D −1, D 1, D 3, D 5, D7 | i.p. | | UUO |
| | | Vehicle | N/A | | |
| | | D 0, D 2, D 4, D 6 | i.p. | | |
| n = 10 | Tg | B5-15 | 10 mg/kg | Once at D 0 | Permanent |
| (3464-3473) | | D −1, D 1, D 3, D 5, D 7 | i.p. | | UUO |
| | | Vehicle | N/A | | |
| | | D 0, D 2, D 4, D 6 | i.p. | | |

Study Endpoints: Morphology: Body (initial, final, A); Kidney weight (obstructed and contralateral) and index; and Tibia length.

Renal Cortical mRNA Expression via Luminex:
Connective Tissue Growth Factor (CTGF)
α-Smooth Muscle Actin (ACTA2)
Fibronectin-1 (FN1)
Collagen 1a1 (Col1a1)
Collagen 3a1 (Col3a1)

Renal Cortical Hydroxyproline Content

Histology readout: Picrosirius Red (PRS) and αvβ8 staining.

The results of IHC staining of kidney tissue of humanized αvβ8 transgenic mice with anti-αvβ8 integrin antibodies to determine kidney fibrosis and the extent thereof are shown in FIGS. 9A-9D. The photomicrographs of IHC staining with anti-αvβ8 integrin antibody as shown in FIGS. 9A and 9B demonstrate that humanized αvβ8 transgenic mice expressed αvβ8 integrin mainly in the glomerulus of the kidney, similar to what is typically observed in healthy human kidney. The induction of fibrosis with the UUO procedure was demonstrated to increase αvβ8 integrin expression in the kidney tubules (FIGS. 9C and 9D), similar to what is typically observed in the kidneys of humans having CKD. FIGS. 9E-9H illustrate the results obtained from the in vivo studies using the UUO procedure as described above and as outlined in Table 3.

As shown in FIG. 9E, the anti-αvβ8 integrin antibodies Chi-37E1B5 (labelled as Parental Avb8 Ab) and B5-15 (labelled as Lead Avb8 Ab) attenuated UUO-induced increases in Col1a1 mRNA expression at 8-days post-UUO surgery relative to UUO controls. As shown in FIG. 9F, the anti-αvβ8 integrin antibodies Chi-37E1B5 (labelled as Parental Avb8 Ab) and B5-15 (labelled as Lead Avb8 Ab) attenuated UUO-induced increases in Col3a1 expression at 8-days post-UUO surgery relative to UUO controls. As shown in FIG. 9G, UUO increased obstructed kidney cortical fibronectin 1 (FN-1) mRNA expression at 8-days post-UUO surgery relative to sham controls. The Chi-37E1B5 (labelled as Parental Avb8 Ab) and B5-15 (labelled as Lead Avb8 Ab) anti-αvβ8 integrin antibodies attenuated UUO-induced increases in FN-1 expression at 8-days of injury duration compared to UUO controls. As shown in FIG. 9H, the anti-αvβ8 integrin antibody B5-15 (labelled as Lead Avb8 Ab) attenuated a UUO-induced increase in α-smooth muscle actin (α-SMA) mRNA expression at 8-days post-UUO surgery relative to UUO controls. The Chi-37E1B5 (labelled as Parental Avb8 Ab) antibody did not reduce the UUO-induced increase in α-SMA. A reduction in α-SMA is important as the presence of α-SMA+ cells is deleterious to normal kidney function. This is because these cells are contractile, directly contributing to the fibrotic remodeling, as well as being highly synthetic, producing pro-inflammatory and pro-fibrotic mediators. As shown in FIG. 9I, the anti-αvβ8 integrin antibodies Chi-37E1B5 (labelled as Parental Avb8 Ab) and B5-15 (labelled as Lead Avb8 Ab) attenuated UUO-induced increases in connective tissue growth factor (CTGF) expression at 8-days post-UUO surgery relative to UUO controls. As shown in FIG. 9J, UUO increased obstructed kidney cortical % hydroxyproline (OH—P) at 8-days post-UUO surgery. The Chi-37E1B5 (labelled as Parental Avb8 Ab) and B5-15 (labelled as Lead Avb8 Ab) antibodies attenuated UUO-induced increases in % OH—P at 8-days UUO injury duration compared to controls. Renal cortical hydroxyproline readout serves as a measurement of actual fibrotic content/fibrosis of tissue.

In summary, at 8-days post-UUO surgery, Chi-37E1B5 and B5-15 attenuated UUO-induced increases in Col1a1, Col3a1, FN-1 and CTGF mRNA expression and % hydroxyproline content. Additionally, at 8-days post-UUO surgery, B5-15 attenuated a UUO-induced increase in α-SMA mRNA expression.

The two anti-αvβ8 integrin antibodies used in this Example, Chi-37E1B5 and B5-15, were administered to the mice in the UUO model at maximal dose. The purpose of this study was to demonstrate whether an antibody against αvβ8 integrin could effectively reduce TGF-β-induced fibrosis caused by association with the αvβ8 integrin. We would expect to see a difference in the reduction of TGF-β-induced fibrosis at lower doses (i.e. EC₅₀) of either of these anti-αvβ8 integrin antibodies. That is, we would expect to see a greater reduction in TGF-β-induced fibrosis in the UUO model from treatment with B5-15 than with Chi-37E1B5 at an equivalent dose. This is primarily because B5-15 demonstrates a greater binding affinity for the αvβ8 integrin than Chi-37E1B5 (see FIG. 1B and FIG. 4) and because B5-15 has greater in vitro potency than Chi-37E1B5 (see FIG. 5). Treatment with B5-15 is advantageous over Chi-37E1B5 because this would achieve less frequent patient dosing or administration of lower doses to patients, leading to fewer, if any, adverse events and greater patient compliance.

As discussed supra, the αvβ8 integrin target receptor is preferentially and highly expressed in diseased/fibrotic kidney tissue and is bound in kidney tissue by the anti-αvβ8 integrin antibody, which interferes with the binding interaction of αvβ8 integrin to latent TGF-β. The anti-αvβ8 integrin antibodies as disclosed herein are particularly advantageous and beneficial for treating fibrotic kidney disease in subjects having kidney disease because use of an antibody directed against the αvβ8 integrin, which binds latent TGF-β, obviates and avoids the targeting of systemic TGF-β, and thus avoids potentially serious problems that could accompany a systemic inhibition of TGF-β in other tissues in the subject undergoing treatment.

### Example 7

### Ex Vivo Studies Using the B5-15 Anti-αvβ8 Integrin Antibody

To evaluate the binding and engagement of the anti-αvβ8 integrin antibody (B5-15) with the target αvβ8 integrin receptor of TGF-β, the activation of the downstream TGF-β signaling pathway was assessed in kidney lysates by measuring total and phosphorylated kidney SMAD2/3. In brief, kidney samples from the animals used in the study described in Example 5 were homogenized in a specific lysis buffer (1× diluted in distilled water+10 μl/ml of protease and phosphatase inhibitor) using a TissueLyser II; protein content was measured using a bicinchoninic acid (BCA) assay as known to and used by those skilled in the art; and protein concentration was normalized for all samples. The total and phosphorylated forms of SMAD2/3 protein (phospho-SMAD2 (Ser465/467)/SMAD3 (Ser423/425)) were analyzed by ELISA following the manufacture's protocol. As noted supra, members of the Smad family of signal transduction molecules are components of the intracellular pathway that transmits TGF-β signals from the cell surface into the nucleus.

The results of the experiments showed that the B5-15 anti-αvβ8 integrin antibody reduced the downstream TGF-β signaling pathway in the kidneys of transgenic mice carrying the human αvβ8-encoding gene (“humanized αvβ8 transgenic mice”) that had undergone unilateral ureteral occlusion (UUO) of 5 days' duration. In FIG. 10A and FIG. 10B, *=<0.05 and ****=≤0.0001. In FIG. 10A and FIG. 10B, for “Sham+NIP228 (IgG isotype control), n=6; for “UUO+NIP228 (IgG isotype control),” n=8; and for “UUO+B5-15 (the anti-αvβ8 integrin antibody),” n=8.

As observed in FIGS. 10A and 10B, UUO surgery in humanized αvβ8 mice resulted in an increase in TGF-β-dependent SMAD2/3 phosphorylation by 5.7-fold versus the Sham-treated group. Of interest, the anti-αvβ8 integrin antibody (B5-15) significantly diminished SMAD2/3 activation by 1.6-fold compared to treatment with the isotype control. Total levels of SMAD2/3 were increased in all UUO groups compared to Sham-treated animals.

### Example 8

### Treatment of a Tri-Culture Cell System Using B5-15, an Anti-αvβ8 Integrin Antibody

To evaluate the effect of B5-15 (an anti-αvβ8 integrin antibody) on a model of human glomerulosclerosis (described in Waters et al., 2017, J Pathol, 243(3):390-400), we treated the tri-culture cell system (where glomerular endothelial cells, podocytes, and mesangial cells form a vascular network) with 10 ng/ml TGF-β or 25 ng/ml CTGF to induce fibrosis. An increase in nodule number is reflective of progression of fibrosis. Treatment with 15 μg/ml of B5-15 significantly reduced nodule number in comparison to treatment with 15 μg/ml of an isotype control (NIP228), see FIG. 11.

### 3D Tri-Culture Formation

In tri-culture human podocytes (Celprogen, CA, USA), glomerular endothelial cells (GECs) and mesangial cells (MCs) (both GECs and MCs from ScienCell Research Laboratories, CA, USA) were suspended within rat tail type 1 collagen (1.5 mg/ml; Corning, Mass., USA), human plasma fibronectin (90 μg/ml; Merck Millipore, Mass., USA), 1.5 mg/ml NaHCO₃, 25 Mm HEPES and M199 medium (10×; Sigma, Mo., USA) at 4° C. Gel was pH adjusted with 0.1M HCl (Fisher Scientific, UK) to pH 7.4. The cell/gel suspension was pipetted into 48 well plates (Corning Incorporated, NY, USA) in a volume of 320 μl per well, respectively. Renal glomerular cells were used at a ratio of 16:3:1 (GECs:PODs:MCs), 330,000-340,000 GECs, 50,000-70,000 PODs and 20,000-24,000 MCs per 320 μl. Cell/gel suspension was polymerised at 37° C. for 20 minutes, after which 500 μl of media was pipetted on top of the gel. Tri-culture media was composed of RMPI 1640 (Gibco™ by Thermo Fisher, UK), 2% FBS, 1% penicillin/streptomycin, 1% insulin, Apo-transferrin, sodium selenite (in ITS mix) and 1% ECGS (supplements all from ScienCell Research Laboratories, CA, USA). Cultures were maintained for 24 hrs. Cells were used in experiments between p2-p6.

Stimulation Assays with TGF-β, NIP228, an Anti-αvβ8 Antibody and CTGF

For stimulation 10 ng/ml TGF-β (R&D Systems (Bio-Techne Ltd), MN, USA), 15 μg/ml NIP228, 15 μg/ml an anti-αvβ8 integrin antibody and 25 ng/ml CTGF (Invitrogen, CA, USA) alone or in combination, were added to media placed on top of culture gels for 24-hour incubation. Control treatment was media alone.

We demonstrate that treatment with an anti-αvβ8 integrin antibody can inhibit the progression of fibrosis caused by TGF-β activation.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of treating kidney fibrosis in a subject having kidney disease, the method comprising administering to the subject an effective amount of an anti-αvβ8 integrin antibody or an antigen binding fragment thereof, thereby treating kidney fibrosis.

2. A method of reducing or attenuating kidney fibrosis in a subject having kidney disease, the method comprising administering to a subject in need thereof an effective amount of an anti-αvβ8 integrin antibody or an antigen-binding fragment thereof, thereby reducing or attenuating fibrosis in the kidney.

3. A method of abrogating the activity of αvβ8 integrin associated with kidney fibrosis, the method comprising administering to a subject in need thereof an effective amount of an anti-αvβ8 integrin antibody or an antigen-binding fragment thereof, thereby abrogating the activity of αvβ8 integrin associated with kidney fibrosis.

4. A method of treating kidney fibrosis by blocking the activation of TGF-β from its latent form in kidney cells and tissue, the method comprising administering to a subject in need thereof an effective amount of an anti-αvβ8 integrin antibody or an antigen-binding fragment thereof, thereby treating the kidney fibrosis.

5. A method of treating kidney damage characterized by an increase in plasma creatinine and/or urinary protein excretion levels, the method comprising administering to a subject in need thereof an effective amount of an anti-αvβ8 integrin antibody or an antigen binding fragment thereof, wherein said administration of the anti-αvβ8 integrin antibody or an antigen binding fragment thereof abrogates the plasma creatinine and/or urinary protein excretion levels in the subject, thereby treating kidney damage.

6. The method of any one of claims 3-5, wherein the subject has kidney disease.

7. The method of any one of claims 1-6, wherein the kidney disease is selected from diabetic nephropathy (DN), chronic kidney disease (CKD), acute kidney disease, hypertension-associated kidney disease, hyperglycemia-associated kidney disease, renal fibrosis, inflammation-associated kidney disease, end stage renal disease (ESRD), autoimmune-associated kidney fibrosis (for example, lupus nephritis) and fibrosis post-kidney transplant.

8. The method of claim 7, wherein the kidney disease is CKD.

9. The method of any one of claims 1-8, wherein the antibody or an antigen binding fragment thereof binds to αvβ8 integrin expressed on kidney cells and/or tissue and blocks the activation of TGF-β from its latent form in the kidney cell and/or tissue.

10. A method of detecting kidney fibrosis in kidney tissue, the method comprising contacting kidney tissue with an effective amount of a detectably labeled anti-αvβ8 integrin antibody or an antigen binding fragment thereof, thereby detecting the binding of the anti-αvβ8 integrin antibody to αvβ8 integrin in the kidney tissue.

11. The method of any one of claims 1-10, wherein the anti-αvβ8 integrin antibody, or an antigen-binding fragment thereof, comprises:
(a) a heavy chain variable region complementarity determining region 1 (CDR1) comprising the amino acid sequence:
RYWMS;
(b) a heavy chain variable region complementarity determining region 2 (CDR2) CDR2 comprising the amino acid sequence:
EINPDSSTINYTSSL;
and
(c) a heavy chain variable region complementarity determining region 3 (CDR3) CDR3 comprising the amino acid sequence:
LITTEDY;
and
(d) a light chain variable region CDR1 comprising the amino acid sequence:
KASQDINSYLS;
(e) a light chain variable region CDR2 comprising the amino acid sequence:
YANRLVD;
and
(f) a light chain variable region CDR3 comprising the amino acid sequence:
LQYDEFPYT.

12. The method of claim 11, wherein the anti-αvβ8 integrin antibody, or an antigen-binding fragment thereof, comprises a heavy chain variable region (V_{H}) amino acid sequence:
EVQLVESGGGLVQPGGSLRLSCAVSGFVFSRYWMSWVRQAPGKGLEWIGE
INPDSSTINYTSSLKDRFTISRDNAKNSLYLQMNSLRAEDTAVYYCAILI
TTEDYWGQGTTVTVSS;
and
a light chain variable region (V_{L}) amino acid sequence:
DIQLTQSPSSLSASVGDRVTITCKASQDINSYLSWFQQKPGKAPKSLIYY
ANRLVDGVPSRFSGSGSGTDFTLTISSLQPEDFATYYCLQYDEFPYTFGG
GTKVEIK.

13. The method of any one of claims 1-10, wherein the anti-αvβ8 integrin antibody, or an antigen-binding fragment thereof, comprises:
(a) a heavy chain variable region CDR1 comprising the amino acid sequence:
RSWIS;
(b) a heavy chain variable region CDR2 comprising the amino acid sequence:
EINPDSSTINYTSSL;
and
(c) a heavy chain variable region CDR3 comprising the amino acid sequence:
LITTEDY;
and
(d) a light chain variable region CDR1 comprising the amino acid sequence:
KASQDINKYLS;
(e) a light chain variable region CDR2 comprising the amino acid sequence:
YANRLVD;
and
(f) a light chain variable region CDR3 comprising the amino acid sequence:
LQYDVFPYT.

14. The method of claim 13, wherein the anti-αvβ8 integrin antibody, or an antigen-binding fragment thereof, comprises a heavy chain variable region (V_{H}) amino acid sequence:
EVQLVESGGGLVQPGGSLRLSCAVSGFVFSRSWISWVRQAPGKGLEWIGE
INPDSSTINYTSSLKDRFTISRDNAKNSLYLQMNSLRAEDTAVYYCAILI
TTEDYWGQGTTVTVSS
and a light chain variable region (V_{L}) amino acid sequence:
DIQLTQSPSSLSASVGDRVTITCKASQDINKYLSWFQQKPGKAPKSLIYY
ANRLVDGVPSRFSGSGSGTDFTLTISSLQPEDFATYYCLQYDVFPYTFGG
GTKVEIK.

15. The method of any one of claims 1-14, wherein the antibody, or an antigen binding fragment thereof, attenuates or abrogates fibrosis associated with increased expression of αvβ8 integrin in podocytes and interstitial tubule cells in kidney tissue of the subject with kidney disease.

16. The method of any one of claims 1-9, or 11-15, wherein the antibody or an antigen binding fragment thereof, is administered to the subject in combination with an adjunct therapeutic agent or treatment for kidney disease.

17. The method of claim 16, wherein the antibody or an antigen binding fragment thereof, is administered to the subject prior to, at the same time as, or after the administration of the adjunct therapeutic agent or treatment.

18. An anti-αvβ8 integrin antibody, or an antigen binding fragment thereof, comprising:
(a) a heavy chain variable region CDR1 comprising the amino acid sequence:
RYWMS;
(b) a heavy chain variable region CDR2 comprising the amino acid sequence:
EINPDSSTINYTSSL;
and
(c) a heavy chain variable region CDR3 comprising the amino acid sequence:
LITTEDY;
and
(d) a light chain variable region CDR1 comprising the amino acid sequence:
KASQDINSYLS;
(e) a light chain variable region CDR2 comprising the amino acid sequence:
YANRLVD;
(f) a light chain variable region CDR3 comprising the amino acid sequence:
LQYDEFPYT.

19. The anti-αvβ8 integrin antibody or an antigen binding fragment thereof of claim 18, comprising a heavy chain variable region (V_{H}) amino acid sequence:
EVQLVESGGGLVQPGGSLRLSCAVSGFVFSRYWMSWVRQAPGKGLEWIGE
INPDSSTINYTSSLKDRFTISRDNAKNSLYLQMNSLRAEDTAVYYCAILI
TTEDYWGQGTTVTVSS;
and
a light chain variable region (V_{L}) amino acid sequence:
DIQLTQSPSSLSASVGDRVTITCKASQDINSYLSWFQQKPGKAPKSLIYY
ANRLVDGVPSRFSGSGSGTDFTLTISSLQPEDFATYYCLQYDEFPYTFGG
GTKVEIK.

20. An anti-αvβ8 integrin antibody or an antigen binding fragment thereof, comprising:
(a) a heavy chain variable region CDR1 comprising the amino acid sequence RSWIS;
(b) a heavy chain variable region CDR2 comprising the amino acid sequence EINPDSSTINYTSSL;
(c) a heavy chain variable region CDR3 comprising the amino acid sequence LITTEDY; and
(d) a light chain variable region CDR1 comprising the amino acid sequence KASQDINKYLS;
(e) a light chain variable region CDR2 comprising the amino acid sequence YANRLVD; and
a light chain variable region CDR3 comprising the amino acid sequence LQYDVFPYT.

21. The anti-αvβ8 integrin antibody or an antigen binding fragment thereof of claim 20, comprising a heavy chain variable region (V_{H}) amino acid sequence:
EVQLVESGGGLVQPGGSLRLSCAVSGFVFSRSWISWVRQAPGKGLEWIGE
INPDSSTINYTSSLKDRFTISRDNAKNSLYLQMNSLRAEDTAVYYCAILI
TTEDYWGQGTTVTVSS
and a light chain variable region (V_{L}) amino acid sequence:
DIQLTQSPSSLSASVGDRVTITCKASQDINKYLSWFQQKPGKAPKSLIYY
ANRLVDGVPSRFSGSGSGTDFTLTISSLQPEDFATYYCLQYDVFPYTFGG
GTKVEIK.

22. An anti-αvβ8 integrin antibody or an antigen binding fragment thereof that competes for binding to αvβ8 integrin with the antibody or an antigen binding fragment thereof of any one of claims 18-21.

23. The antibody or an antigen binding fragment thereof of any one of claims 18-22, for use in a method of treating kidney fibrosis, wherein said antibody or an antigen binding fragment thereof specifically binds to αvβ8 integrin, thereby treating kidney fibrosis.

24. The antibody or an antigen-binding fragment thereof of claim 23, wherein said antibody or an antigen-binding fragment thereof specifically binds to αvβ8 integrin expressed on fibrotic kidney cells and tissue and blocks binding of αvβ8 integrin to latent TGF-β, thereby abrogating the activity of αvβ8 integrin associated with kidney fibrosis to treat kidney disease.

25. A polynucleotide encoding the antibody or an antigen binding fragment thereof of claim 18 or claim 19.

26. A polynucleotide encoding the antibody or an antigen binding fragment thereof of claim 20 or claim 21.

27. The polynucleotide of claim 26, wherein the V_{H} region coding sequence comprises nucleic acid sequence:
gaggtgcagctggtggaaagcggcggaggactggtgcagcctggcggcag
cctgagactgagctgcgccgtgtccggcttcgtgttcagccggagctgga
tcagctgggtccgccaggccccagggaagggcctggaatggatcggcgag
atcaaccccgacagcagcaccatcaactacaccagcagcctgaaggaccg
gttcaccatcagccgggacaacgccaagaacagcctgtacctgcagatga
acagcctgcgggccgaggacaccgccgtgtactactgcgccatcctcatc
accaccgaggactactggggccagggcaccaccgtgaccgtgtcctct;
and the V_{L} region coding sequence comprises nucleic acid sequence:
gacatccagctgacccagagccccagcagcctgagcgccagcgtgggcga
cagagtgaccatcacatgcaaggccagccaggacatcaacaagtacctga
gctggttccagcagaagcccggcaaggcccccaagagcctgatctactac
gccaaccggctggtggacggcgtgcccagcagattttctggcagcggcag
cggcaccgacttcaccctgaccatcagcagcctgcagcccgaggacttcg
ccacctactactgcctgcagtacgacgtgttcccctacaccttcggcgga
ggcaccaaggtggaaatcaag.

28. An expression vector which comprises the polynucleotide of any one of claims 25-27.

29. The expression vector of claim 28, which is a prokaryotic, eukaryotic, or mammalian expression vector.

30. A cell comprising the expression vector of claim 28 or claim 29.

31. The cell of claim 30, which is a prokaryotic, a eukaryotic, or a mammalian host cell.

32. A pharmaceutical composition comprising the antibody or an antigen-binding fragment thereof of any one of claims 18-24, and a pharmaceutically acceptable carrier, excipient, or diluent.

33. A pharmaceutical composition comprising the polynucleotide of any one of claims 25-27, and a pharmaceutically acceptable carrier, excipient, or diluent.

34. A kit comprising the antibody or an antigen binding fragment thereof that specifically binds to αvβ8 integrin of any one of claims 18-24, or a pharmaceutical composition comprising the antibody or the antigen binding fragment thereof.
